(19) United States
(12) Patent Application Publication
(10) Pub. No.: US 2023/0395185 A1
(43) Pub. Date: Dec. 7, 2023

(54) SYSTEMS FOR AND METHODS OF DETERMINING PROTEIN-PROTEIN INTERACTION

(71) Applicant: THE REGENTS OF THE UNIVERSITY OF CALIFORNIA, Oakland, CA (US)

(72) Inventors: Nevan J. KROGAN, San Francisco, CA (US); Hannes BRABERG, San Francisco, CA (US)

(21) Appl. No.: 18/032,159

(22) PCT Filed: Oct. 14, 2021
(86) PCT No.: PCT/US2021/055059

§ 371 Date: Apr. 14, 2023

Related U.S. Application Data
(60) Provisional application No. 63/091,916, filed on Oct. 14, 2020.

(51) Int. Cl. G16B 15/00; G16B 5/20; G16B 20/20; G16B 25/10

(52) U.S. Cl. CPC G16B 15/00; G16B 5/20; G16B 20/20; G16B 25/10

(57) ABSTRACT

The disclosure relates to a system comprising software that predicts a protein-protein interaction associated with a disorder. Embodiments of the disclosure include methods comprising mapping a protein-protein interaction as between a first amino acid sequence comprising a point mutation relative to a second amino acid sequence which is a wild-type sequence relative to the first amino acid sequence and a third amino acid sequence.

### CROSS-REFERENCE TO RELATED APPLICATIONS

This application claims the benefit of U.S. Application No. 63/091,916, filed on Oct. 14, 2020, the contents of which are hereby incorporated by reference in their entirety.

### STATEMENT REGARDING FEDERALLY SPONSORED RESEARCH

This invention was made with government support under grants R01 GM084448, R01 GM084279, P50 GM081879, R01 GM098101, P50 A1150476, P50 GM082250, U19 A1135990, P01 HL089707, and NS100717 awarded by The National Institutes of Health. The government has certain rights in the invention.

### FIELD OF INVENTION

The disclosure relates to a system comprising software that predicts a protein-protein interaction associated with a disorder. This system can be used for, for example, developing protein-protein interaction maps, identifying protein-protein interactions, identifying therapeutic targets, and screening for and evaluating therapeutics. Embodiments of the disclosure include methods comprising mapping a protein-protein interaction as between a first amino acid sequence comprising a point mutation relative to a second amino acid sequence which is a wild-type sequence relative to the first amino acid sequence and a third amino acid sequence.

### BACKGROUND

A mechanistic understanding of cellular functions requires structural characterization of the corresponding macromolecular assemblies (1). Traditional structural biology methods, such as X-ray crystallography, nuclear magnetic resonance (NMR) spectroscopy, and electron microscopy (EM), rely on purified samples and are generally not applicable to heterogeneous samples, such as those of large, membrane-bound, or transient assemblies (2). Moreover, these methods do not determine the structures in their native environments, therefore increasing the risk of producing structures in non-functional states or missing relevant functional states.

Integrative structure determination has emerged as a powerful approach for determining the structures of biological assemblies (3). The motivation behind the integrative approach is deceptively simple: namely, any system can be described most accurately, precisely, completely, and efficiently by using all available information about it, including varied experimental data (e.g. chemical cross-links, protein interaction data, small-angle X-ray scattering profiles) and prior models (e.g. atomic structures of the subunits). Integrative methods can often tackle protein assemblies that are difficult to characterize using traditional structural biology methods alone (1, 4-10). Spatial data generated by in vivo methods is especially useful for integrative structure determination (11). Therefore, high-throughput in vivo methods are needed to supplement low-throughput in vivo methods, such as single molecule Forster resonance energy transfer (FRET) spectroscopy (12).

### SUMMARY OF EMBODIMENTS

Described here is an integrative structure determination approach that relies on in vivo measurements of genetic interactions. We construct phenotypic profiles for point mutations crossed against gene deletions or exposed to environmental perturbations, followed by converting similarities between two profiles into an upper bound on the distance between the mutated residues. To enable this integrative approach, we generate ˜500,000 genetic interactions of 350 mutants in yeast histones H3 and H4. We then apply the method to the histones, subunits Rpb1-Rpb2 of yeast RNA polymerase II, and subunits RpoB-RpoC of bacterial RNA polymerase. The accuracy is comparable to that based on chemical cross-links; using restraints from both genetic interactions and cross-links further improves model accuracy and precision. The approach can be readily applied to other systems, providing an efficient means to augment integrative structure determination with in vivo observations.

In some embodiments, the disclosure relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic miniarray profile (pE-MAP) or a chemical genetics miniarray profile (CG-MAP) associated with the first nucleic acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

In some embodiments, the disorder affects regulation of amino acid expression in a subject.

In some embodiments, the disorder is a cancer.

Some embodiments further comprise a step of performing a functional bioassay to display phenotype of a cell or subject expressing the first nucleic acid sequence relative to expression of the second nucleic acid sequence.

In some embodiments the functional bioassay comprises a yeast two-hybrid screen and/or mass spectrometry.

In some embodiments, the correlating of step (e) comprises calculating a Pearson correlation.

In some embodiments, the correlating of step (e) comprises: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the upper distance bound is calculated by: (a) binning the plurality of MICs into 20 intervals; (b) selecting a maximum distance spanned by any pair of amino acid residues in each bin; and (c) fitting a logarithmic decay function (dᵤ) to the upper distance bound:

$${d}_{U} ( M I C ) = \{ \begin{matrix}\frac{log ( M I C ) - n}{n} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M|I)

wherein the model, M, consists of a structure X and unknown parameters Y; prior p(M|I) is the probability density of model M given I; and likelihood function p(D|M, I) is the probability density of observing data D given M and I.

Some embodiments further comprise calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein f_{i,j}(X) is a forward model that predicts the data point d_{i,j} in D that would have been observed for structure Xin an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

wherein d₀=dᵤ (0.6); wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:

$$p ( M I {C}_{i , j}^{obs} [ M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σ_{i,j} are the noise parameters that can optionally be determined as part of the model, and N and M are normalization factors necessary to make the likelihood continuous.

Some embodiments further comprise obtaining a Bayesian term in the scoring function defining as the negative logarithm of the posterior probability density:

S(M)=−log p(M|D,I).

In some embodiments, the mapping of step (f) comprises performing a structure E-MAP (stE-MAP) as between expression of the first nucleic acid sequence and the third nucleic acid sequence.

In some embodiments, the mapping of the protein-protein interaction has a resolution from about 1 to about 10 angstroms.

In some embodiments, the disclosure relates to a method of determining structure of a protein-protein interaction, said method comprising: (a) selecting a first nucleic acid sequence and a second nucleic acid sequence, wherein the second nucleic acid sequence is a wild-type sequence relative to the first nucleic acid sequence, and wherein at least the first nucleic acid sequence comprises a point mutation relative to the second nucleic acid sequence; (b) creating a point-mutant epistatic miniarray profile (pE-MAP) or a chemical genetics miniarray profile (CG-MAP) associated with the first nucleic acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

In some embodiments, the correlating of step (e) comprises calculating a Pearson correlation.

In some embodiments, the correlating of step (e) comprises: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the upper distance bound is calculated by: (a) binning the plurality of MICs into 20 intervals; (b) selecting a maximum distance spanned by any pair of amino acid residues in each bin; and (c) fitting a logarithmic decay function (dᵤ) to the upper distance bound:

$${d}_{U} ( M I C ) = \{ \begin{matrix}\frac{log ( M I C ) - n}{k} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M|I)

wherein the model, M, consists of a structure X and unknown parameters Y; prior p(M|I) is the probability density of model M given I; and likelihood function p(D|M, I) is the probability density of observing data D given M and I.

Some embodiments further comprise calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein f_{i,j}(X) is a forward model that predicts the data point d_{i,j} in D that would have been observed for structure Xin an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

wherein d₀=dᵤ (0.6); wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:

$$P ( M I {C}_{i , j}^{obs} \vert M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σ_{i,j} are the noise parameters that can optionally be determined as part of the model, and N and M are normalization factors necessary to make the likelihood continuous.

Some embodiments further comprise obtaining a Bayesian term in the scoring function defining as the negative logarithm of the posterior probability density:

S(M)=−log p(M|D,I).

In some embodiments, the mapping of step (f) comprises performing a structure E-MAP (stE-MAP) as between expression of the first nucleic acid sequence and the third nucleic acid sequence.

In some embodiments, the mapping of the protein-protein interaction has a resolution from about 1 to about 10 angstroms.

In some embodiments, the disclosure relates to a method of modeling a three-dimensional structure as between two or more amino acid sequences, said method comprising: (a) selecting a first amino acid sequence and a second amino acid sequence, wherein the second amino acid sequence is a wild-type sequence relative to the first amino acid sequence, and wherein at least the first amino acid sequence comprises a point mutation relative to the second amino acid sequence; (b) creating a point-mutant epistatic miniarray profile (pE-MAP) or a chemical genetics miniarray profile (CG-MAP) associated with the first amino acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within the first amino acid sequence; and (ii) spatial positions of amino acid residues within the third amino acid sequence; and (f) mapping a protein-protein interaction as between the first amino acid sequence and the third amino acid sequence.

In some embodiments, the correlating of step (e) comprises calculating a Pearson correlation.

In some embodiments, the correlating of step (e) comprises: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the upper distance bound is calculated by: (a) binning the plurality of MICs into 20 intervals; (b) selecting a maximum distance spanned by any pair of amino acid residues in each bin; and (c) fitting a logarithmic decay function (dᵤ) to the upper distance bound:

$${d}_{U} ( M I C ) = \{ \begin{matrix}\frac{log ( M I C ) - n}{k} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M|I)

wherein the model, M, consists of a structure X and unknown parameters Y; prior p(M|I) is the probability density of model M given I; and likelihood function p(D|M, I) is the probability density of observing data D given M and I.

Some embodiments further comprise calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein f_{i,j}(X) is a forward model that predicts the data point d_{i,j} in D that would have been observed for structure Xin an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

wherein d₀=dᵤ (0.6); wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:

$$P ( M I {C}_{i , j}^{obs} \vert M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σ_{i,j} are the noise parameters that can optionally be determined as part of the model, and N and M are normalization factors necessary to make the likelihood continuous.

Some embodiments further comprise obtaining a Bayesian term in the scoring function defining as the negative logarithm of the posterior probability density:

S(M)=−log p(M|D,I).

In some embodiments, the mapping of step (f) comprises performing a structure E-MAP (stE-MAP) as between expression of the first nucleic acid sequence and the third nucleic acid sequence.

In some embodiments, the mapping of the protein-protein interaction has a resolution from about 1 to about 10 angstroms.

In some embodiments, the disclosure relates to a computer program product encoded on a computer-readable storage medium, wherein the computer program product comprises instructions for: (a) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence; (b) calculating an S-score associated with the first nucleic acid sequence and the third nucleic acid sequence; (c) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and (d) mapping a protein-protein interaction as between the amino acid sequence encoded by the first nucleic acid sequence and the amino acid sequence encoded by the third nucleic acid sequence.

In some embodiments, the correlating of step (e) comprises: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the upper distance bound is calculated by: (a) binning the plurality of MICs into 20 intervals; (b) selecting a maximum distance spanned by any pair of amino acid residues in each bin; and (c) fitting a logarithmic decay function (dᵤ) to the upper distance bound:

$${d}_{U} ( M I C ) = \{ \begin{matrix}\frac{log ( M I C ) - n}{k} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M,I)

wherein the model, M, consists of a structure X and unknown parameters Y; prior p(M|I) is the probability density of model M given I; and likelihood function p(D|M, I) is the probability density of observing data D given M and I.

Some embodiments further comprise calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein f_{i,j}(X) is a forward model that predicts the data point d_{i,j} in D that would have been observed for structure Xin an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

wherein d₀=dᵤ (0.6); wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:

$$P ( M I {C}_{i , j}^{obs} \vert M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σ_{i,j} are the noise parameters that can optionally be determined as part of the model, and N and M are normalization factors necessary to make the likelihood continuous.

Some embodiments further comprise obtaining a Bayesian term in the scoring function defining as the negative logarithm of the posterior probability density:

S(M)=−log p(M|D,I).

In some embodiments, the mapping of step (f) comprises performing a structure E-MAP (stE-MAP) as between expression of the first nucleic acid sequence and the third nucleic acid sequence.

Some embodiments further comprise instructions for correlating a differential interaction score (DIS) with a likelihood that the dysfunctional protein-protein interaction is a causal agent of a disorder.

Some embodiments further comprise instructions for selecting a treatment for a subject based upon the causal agent.

Some embodiments of a system comprising the computer program product further comprise one or more of: (a) a processor operable to execute programs; and (b) a memory associated with the processor.

In some embodiments, the disclosure relates to a system for identifying a protein interaction network in a subject, the system comprising: (a) a processor operable to execute programs; (b) a memory associated with the processor; (c) a database associated with said processor and said memory; and (d) a program stored in the memory and executable by the processor, the program being operably for: (i) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence; (ii) calculating an S-score associated with the first nucleic acid sequence and the third nucleic acid sequence; (iii) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and (iv) mapping a protein-protein interaction as between the amino acid sequence encoded by the first nucleic acid sequence and the amino acid sequence encoded by the third nucleic acid sequence.

In some embodiments, the disclosure relates to a method of modeling a protein-protein interaction, said method comprising: (a) calculating a plurality of MICs associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence; (b) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (c) performing a noise model for calculation of a standard deviation; and (d) formulating spatial restraints as Bayesian data likelihoods.

In some embodiments, the upper distance bound is calculated by: (a) binning the plurality of MICs into 20 intervals; (b) selecting a maximum distance spanned by any pair of amino acid residues in each bin; and (c) fitting a logarithmic decay function (dᵤ) to the upper distance bound:

$${d}_{U} ( M I C ) = \{ \begin{matrix}\frac{log ( M I C ) - n}{k} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M|I)

wherein the model, M, consists of a structure X and unknown parameters Y; prior p(M|I) is the probability density of model M given I; and likelihood function p(D|M, I) is the probability density of observing data D given M and I.

In some embodiments, the disclosure relates to a method of modeling a protein-protein interaction, said method comprising: (a) processing genetic interaction phenotypic profiles; (b) devising a phenotypic similarity metric between the phenotypic profiles; and (c) designing spatial restraints for integrative structure modeling.

In some embodiments, the disclosure relates to a method of creating a genetic interaction profile, said method comprising: (a) creating a point-mutant epistatic miniarray profile (pE-MAP) or a chemical genetics miniarray profile (CG-MAP) associated with a first nucleic acid sequence and a second nucleic acid sequence; (b) calculating an S-score associated with the first nucleic acid sequence and the second nucleic acid sequence; and (c) correlating the pE-MAP and the S-score, or the CG-MAP and the S-score, to create the genetic interaction profile between the first nucleic acid sequence and the second nucleic acid sequence.

In some embodiments, the disclosure relates to a method of predicting effect of histone mutation on methylation of a histone protein, said method comprising: (a) creating a point-mutant epistatic miniarray profile (pE-MAP) or a chemical genetics miniarray profile (CG-MAP) associated with the histone protein; (b) calculating an S-score associated with the histone protein; and (c) correlating the pE-MAP and the S-score, or the CG-MAP and the S-score, to create a protein structural model of the histone protein.

In some embodiments, the disclosure relates to a method of identifying perturbations in a protein-protein interaction comprising: (a) mutating one or more nucleic acids in the genome of a cell; (b) analyzing the mutation by devising a phenotypic profile associated with the mutation; wherein the step of analyzing comprises comparing the phenotypic profile associated with the mutation with the phenotypic profile associated with one or more nucleic acids free of the mutation.

In some embodiments, the disclosure relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic miniarray profile (pE-MAP) and/or a chemical genetics miniarray profile (CG-MAP) associated with the first nucleic acid sequence by calculating an S-score associated with the first nucleic acid sequence and a third nucleic acid sequence; (d) calculating a maximal information coefficient (MIC) corresponding to the first nucleic acid sequence; (e) correlating the MIC with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

In some embodiments, the methods further comprise a step of displaying an image of the protein-protein interaction on a display as a component of any of the disclosed systems after the step of mapping is completed.

In some embodiments, the disclosure also relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic miniarray profile (pE-MAP) and/or a chemical genetics miniarray profile (CG-MAP) associated with the first nucleic acid sequence by calculating an S-score associated with the first nucleic acid sequence and a third nucleic acid sequence; (c) calculating a maximal information coefficient (MIC) between the pE-MAP or CG-MAP of the first nucleic acid sequence and the third nucleic acid sequence; (d) correlating the MIC with: (i) spatial positions of amino acid residues within an amino acid sequence encoded by the first nucleic acid sequence; and (ii) spatial positions of amino acid residues within an amino acid sequence encoded by the third nucleic acid sequence; and (e) mapping a protein-protein interaction as between the amino acid sequence encoded by the first nucleic acid sequence and the amino acid sequence encoded by the third nucleic acid sequence.

In some embodiments, the methods further comprise a step of displaying an image of the protein-protein interaction on a display as a component of any of the disclosed systems after the step of mapping is completed.

In some embodiments, the disclosure also relates to a method of determining structure of a protein-protein interaction, said method comprising: (a) selecting a first nucleic acid sequence and a second nucleic acid sequence, wherein the second nucleic acid sequence is a wild-type sequence relative to the first nucleic acid sequence, and wherein at least the first nucleic acid sequence comprises a point mutation relative to the second nucleic acid sequence; (b) creating a point-mutant epistatic miniarray profile (pE-MAP) or a chemical genetics miniarray profile (CG-MAP) associated with the first nucleic acid sequence; (c) calculating a maximal information coefficient (MIC) between the pE-MAP or CG-MAP of the first nucleic acid sequence and the third nucleic acid sequence; (d) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (e) mapping a protein-protein interaction as between the amino acid sequence encoded by the first nucleic acid sequence and the amino acid sequence encoded by the third nucleic acid sequence.

In some embodiments, the disclosure also relates to a method of modeling a three-dimensional structure as between two or more amino acid sequences, said method comprising: (a) selecting a first amino acid sequence and a second amino acid sequence, wherein the second amino acid sequence is a wild-type sequence relative to the first amino acid sequence, and wherein at least the first amino acid sequence comprises a point mutation relative to the second amino acid sequence; (b) creating a point-mutant epistatic miniarray profile (pE-MAP) or a chemical genetics miniarray profile (CG-MAP) associated with the first nucleic acid sequence; (c) calculating a maximal information coefficient (MIC) between the pE-MAP or CG-MAP of the first nucleic acid sequence and the third nucleic acid sequence; (d) correlating the MIC and the pE-MAP, or the MIC and the CG-MAP, with: (i) spatial positions of amino acid residues within the first amino acid sequence; and (ii) spatial positions of amino acid residues within the third amino acid sequence; and (e) mapping a protein-protein interaction as between the first amino acid sequence and the third amino acid sequence.

In some embodiments, the disclosure also relates to a computer program product encoded on a computer-readable storage medium, wherein the computer program product comprises instructions for: (a) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence; (b) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and (c) mapping a protein-protein interaction as between the amino acid sequence encoded by the first nucleic acid sequence and the amino acid sequence encoded by the third nucleic acid sequence.

In some embodiments, the disclosure also relates to a computer program product encoded on a computer-readable storage medium, wherein the computer program product comprises instructions for: (a) generating a pE-MAP or CG-MAP by calculating S-scores associated with a first nucleic acid sequence wherein the first nucleic acid sequence comprises a point mutation relative to a second sequence which is a wild-type sequence relative to the first nucleic acid sequence; (b) calculating a maximal information coefficient (MIC) between the pE-MAP or CG-MAP of the first nucleic acid sequence and a third nucleic acid sequence.

In some embodiments, the step of correlating comprises: (i) calculating a plurality of MICs as between the first sequence and a series of nucleic acid sequences comprising the third nucleic acid sequence; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the disclosure also relates to a system for identifying a protein interaction network in a subject, the system comprising: (a) a processor operable to execute programs; (b) a memory associated with the processor; (c) a database associated with said processor and said memory; and (d) a program stored in the memory and executable by the processor, the program being operably for: (i) calculating a maximal information coefficient (MIC) associated with a first nucleic acid sequence and a second nucleic acid sequence, wherein the first nucleic acid sequence comprises a point mutation relative to a second sequence which is a wild-type sequence relative to the first nucleic acid sequence; (ii) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the second nucleic acid sequence; and (iii) mapping a protein-protein interaction as between the amino acid sequence encoded by the first nucleic acid sequence and the amino acid sequence encoded by the second nucleic acid sequence.

In some embodiments, the method of modeling a protein-protein interaction, said method comprising: (a) calculating a plurality of MICs associated with a first nucleic acid sequence relative to a plurality of nucleic acid sequences free of the first nucleic acid sequence, wherein the first nucleic acid sequence comprises a point mutation relative to a known sequence; (b) calculating an upper distance bound between the spatial positions of the amino acid residues within the amino acid sequence encoded by the first nucleic acid sequence and the spatial positions of the amino acid residues within the amino acid sequence encoded by the plurality of nucleic acid sequences free of the first nucleic acid sequence; (c) performing a noise model for calculation of a standard deviation; and (d) formulating spatial restraints as Bayesian data likelihoods.

In some embodiments, the disclosure also relates to a method of creating a genetic interaction profile, said method comprising: (a) creating a point-mutant epistatic miniarray profile (pE-MAP) and/or a chemical genetics miniarray profile (CG-MAP) associated with a first nucleic acid sequence and a second nucleic acid sequence by calculating an S-score associated with each of the first and second nucleic acid sequences; (b) correlating the genetic or chemical-genetic interaction profiles of the first nucleic acid sequence and the second nucleic acid sequence as a measure of similarity.

In some embodiments, the disclosure also relates to method of predicting effect of histone mutation on post-translational modifications of a histone protein, said method comprising: (a) creating a point-mutant epistatic miniarray profile (pE-MAP) or a chemical genetics miniarray profile (CG-MAP) associated with the histone protein; and (b) correlating the pE-MAP and/or the CG-MAP and the S-score, to a protein structural model of the histone protein; (c) identifying the effect to post-translational modifications by comparing the change in protein-protein interaction in a nucleic acid sequence comprising a mutation as relative to the protein-protein interaction of the same unmutated nucleic acid sequence.

In some embodiments, the method of predicting effect further comprises fitting a model of protein-protein interaction generated by the amount of a nucleic acid or amino acid in a system comprising a therapeutic agent.

In any of the above methods, method the correlating steps may comprise the substeps of: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In any of the above steps, the upper distance bound is calculated by: (a) binning the plurality of MICs into from about 2 to about 1,000 intervals; (b) selecting a maximum distance spanned by any pair of amino acid residues in each bin; and (c) fitting a logarithmic decay function (du) to the upper distance bound:

$${d}_{U} ( M I C ) = \{ \begin{matrix}\frac{log ( M I C ) - n}{k} & if M I C \leq \dot{O} \\ i & if M I C > \dot{O}\end{matrix} \qquad ( 1 )$$

wherein n is a negative number from about 0 and to about −1; wherein k is a negative number between 0 and −1; wherein i is an interval number from about 5 to about 1,000, or from about 10 to about 40; and wherein \dot{O} is from about 0.4 to about 0.8.

In some embodiments, \dot{O} is about 0.4, about 0.5, about 0.6, about 0.7 or about 0.8.

In some embodiments, I is an interval number from about 10 to about 100, from about 20 to about 40, or from about 30 to about 40. In some embodiments, an interval number of about 10, about 20, about 30, about 40 or about 50.

In some embodiments, in any of the disclosed methods, the method further comprises substeps of further comprising calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein fi,j(X) is a forward model that predicts the data point di,j in D that would have been observed for structure X in an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ & if {d}_{i , j} > {d}_{0}\end{matrix} \qquad ( 2 )$$

wherein d0=du (from about 0.4 to about 0.8); and wherein N(di,j|fi,j(X),σi,j) is a noise model that quantifies the deviation between the predicted and observed data points and is a lognormal distribution with a flat plateau for MIC values below the upper bound on the observed MIC values:

$$P ( M I {C}_{i , j}^{obs} \vert M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σi,j are the noise parameters that can optionally be determined as part of the model, and N and M are normalization factors necessary to make the likelihood continuous.

In some embodiments, any of the methods disclosed can include a step of mapping, wherein the mapping of one or a plurality of protein-protein interactions has a resolution from about 1 to about 20 angstroms. In some embodiments, any of the methods disclosed can include a step of mapping, wherein the mapping of one or a plurality of protein-protein interactions has a resolution from about 5 to about 15 angstroms. In some embodiments, any of the methods disclosed can include a step of mapping, wherein the mapping of one or a plurality of protein-protein interactions has a resolution from about 10 to about 20 angstroms.

In some embodiments, any of the methods disclosed can include a step of mapping, wherein the mapping of one or a plurality of protein-protein interactions has a resolution of about 12, about 13, about 14, about 15, about 16, about 17, about 18, about 19 or about 20 angstroms.

### BRIEF DESCRIPTION OF DRAWINGS

The accompanying figures, which are incorporated in and constitute a part of this specification, illustrate several embodiments and together with the description serve to explain the principles of the invention.

FIG. 1A-1G depict building spatial restraints from pairwise genetic perturbations. FIG. 1A: Genetic interactions arise when the combined fitness defect of a double mutant deviates from the expected multiplicative growth defect of the two single mutants. FIG. 1B: The generation of a pE-MAP relies on a collection of point mutations, which is constructed by systematic mutagenesis of genes that encode the subunits of a macromolecular assembly (mutations labeled 1-4). The point mutant strains are then crossed against a library of gene deletions, followed by fitness measurement and subsequent calculation of genetic interaction scores and the resulting phenotypic profiles. FIG. 1C: Each histone mutant strain was modified at both native loci (HHT1 & HHT2 for H3 or HHF1 & HHF2 for H4, red stars) and crossed against a library of 1370 different deletion mutants (or hypomorphic alleles for essential genes). FIG. 1D: Hierarchically clustered pE-MAP of the 350 histone H3 and H4 alleles (FIG. 6), screened against the deletion library. The pE-MAP consists of more than 479,000 genetic interactions. FIG. 1E: Blow-up of the pE-MAP section highlighted in FIG. 1D. When clustering the phenotypic profiles, deletions of genes that function in the same pathway or belong to the same complex often group together (examples labeled on upper x-axis). Likewise, point mutations that have similar functional effects tend to group together in a clustered pE-MAP (y-axis). FIG. 1F: Each pairwise combination of phenotypic profiles is transformed into a single MIC value that reflects the similarity between the two profiles. FIG. 1G: Relationship between pairwise distance and the MIC value. The background color gradient reflects how the score depends on MIC value and distance. The inset shows the score as a function of distance for different MIC values (Methods).

FIG. 2 depicts a description of the integrative modeling workflow. The four stages include: (1) gathering all available experimental data and prior information; (2) translating all information into representations of assembly components and a scoring function for ranking alternative assembly structures; (3) sampling structural models; and (4) validating the model. In this example, representations of the components of a complex are based on comparative models of its components. The scoring function consists of spatial restraints that are obtained from pE-MAP and/or cross-linking experiments (evolutionary coupling analysis is not indicated in this scheme) as well as excluded volume and sequence connectivity restraints. The sampling explores the configurations of rigid components, searching for those assembly structures that satisfy the spatial restraints as well as possible. The goal is to obtain an ensemble of structures that satisfy the input data within the uncertainty of the data used to compute them. The sampling precision is estimated, models are clustered, and evaluated by the degree to which they satisfy the input information used to construct them as well as omitted information. The protocol can iterate through the four stages until the models are judged to be satisfactory, most often based on their precision and the degree to which they satisfy the data.

FIG. 3A-3E depict integrative structure determination of histones H3 and H4. FIG. 3A: The native structure of the histone H3-H4 dimer (PDB: 1ID3, left) and its contact map (right). In contact maps, the intensity of gray is proportional to the relative frequency of residue-residue contacts in the models (cutoff distance of 12 Å). For X-ray structures, the contact frequency is either 0 (white) or 1 (black). The circles correspond to the pairs of restrained residues, with the intensity of red proportional to the MIC value (MIC>0.3), showing that the pairs of residues with high MIC values are distributed throughout the proteins. FIG. 3B: The localization probability density of the ensemble of structures is shown with a representative (centroid) structure from the ensemble embedded within it (left) and the corresponding contact map (right). The localization probability density map represents the probability of any volume element being occupied by a given protein. The intensity of gray is proportional to the contact frequency of models in the ensemble whose distance is closer than the contact cutoff of 12 Å. FIG. 3C: Localization probability density and centroid structure (left), and contact map (right), computed with shuffled MIC values (Methods). FIG. 3D: Distributions of accuracy of structures in the ensembles based on the full pE-MAP dataset, resampled datasets that consider fractions of the data, and using shuffled MIC values. The white dots represent median accuracies. FIG. 3E: Model precision for different realizations using the full pE-MAP dataset, resampled dataset that considers fractions of the data, and using shuffled MIC values. The error bars represent the standard deviations of model precision over three independent realizations (shown as dots).

FIG. 4A-4D depict connecting individual histone residues and regions to other associated complexes. FIG. 4A: Comparison of S-scores and correlations of phenotypic profiles of modifier-residue pairs to the overall data. Only residues with a single known modifier and modifiers with a single known target residue were included (Table 3). FIG. 4B: Average distributions of S-scores (left) and phenotypic profile correlations (right) of H3K4 mutants (mean of H3K4Q and H3K4R). Members of the COMPASS complex that exhibit a mean S-score >2.5 or a mean genetic interaction profile correlation >0.2 with H3K4 mutants are highlighted. To make the plots comparable, both distributions only include the 1,263 gene deletions/hypomorphic alleles that overlap in the pE-MAP and the correlation map. Five of the six COMPASS members are present in this intersection (SHG1 is not). The COMPASS complex is responsible for H3K4 methylation.

FIG. 4C: Mapping of genetic interaction profile correlations to COMPASS complex members on the structure of the nucleosome (modified PDB 1ID3). N-terminal tail residues of H3 and H4 not included in 1ID3 are visualized as strings on the periphery. Only residues that exhibit a median genetic profile correlation >0.2 with the COMPASS subunits are highlighted (Methods). H3 is depicted in purple, H4 in light green, and H2A/H2B and DNA in grey. The red color gradient reflects the strength of the correlation between each residue and the COMPASS members, calculated as the median correlation between the residue's tested mutations and the COMPASS members. FIG. 4D: Distributions of genetic interaction profile correlations of H3K56Q (acetylation mimic) and H3K56R (deacetylation mimic). Correlations of key H3K56ac-level regulators, Rtt109 (acetylating) and Hst3 (deacetylating), are highlighted. The cartoon outlines the H3K56 acetylation pathway and its role in H3 ubiquitylation. Rtt109 acetylates H3K56 via an Asf1-dependent mechanism, which promotes ubiquitylation of H3 by Rtt101-Mms1 and Mms22. These 5 gene deletions are all found among the top 10 most similar to the deacetylation mimic H3K56R, whereas deletion of the H3K56 deacetylase Hst3 instead gives rise to a profile similar to the acetylation mimic H3K56Q (table inset).

FIG. 5A-5H depict integrative structure determination of yeast RNAPII and bacterial RNAP. FIG. 5A: The native structure of Rpb1-Rpb2 (PDB: 1I3Q) showing its three rigid-body components. Rpb1 was split into two domains, as shown. FIG. 5B: The localization probability density of the ensemble of the three rigid-body structures is shown with a representative (centroid) structure from the ensemble embedded within it. FIG. 5C: Contact maps computed for the X-ray structure (top) and model using the pE-MAP dataset (bottom). The circles correspond to the pairs of restrained residues, with the intensity of red proportional to the MIC value (MIC>0.3). FIG. 5D: Distributions of accuracy (top) for all structures in the ensemble and model precisions (bottom) for the computed ensembles based on pE-MAP and cross-link (XL) data. The white dots represent median accuracies. FIG. 5E: Structure of subunits RpoB and RpoC from bacterial RNAP (PDB: 4YC2). FIG. 5F: The localization probability density of the ensemble of the RpoB-RpoC structures with a representative (centroid) structure from the ensemble embedded within it. FIG. 5G: Contact maps computed for the X-ray structure (top) and model using the CG-MAP dataset (bottom). The shaded yellow band represents a region missing in the X-ray structure. FIG. 5H: Distributions of accuracy (top) for all structures in the ensemble and model precisions (bottom) for the ensembles based on CG-MAP and evolutionary coupling (EVC) data. The error bars represent the standard deviations of model precision over three independent realizations (shown as dots). The white dots represent median accuracies.

FIG. 6A-6D depict genetic interrogation of histones H3 and H4 at a residue-level resolution. FIG. 6A: Schematic of the histone point mutants analyzed in this study (Table 1). Secondary structure elements are indicated as ribbons above the amino acid sequence. The mutations are color-coded according to the mutation introduced (FIG. 6B). Mutations resulting in inviable strains or strains too sick for genetic analysis are shown in FIG. 7. FIG. 6B: Table of histone mutant categories and their hypothesized effects (color-coding as in FIG. 6A). FIG. 6C: Overview of viable H3 and H4 tail deletion mutants amenable to pE-MAP analysis. The amino acid sequences of the wt alleles are shown on top (residues 1-39 of histone H3 and 1-27 of histone H4). Grey bars signify the deleted amino acids in H3 and H4. FIG. 6D: Reproducibility of histone pE-MAP S-scores between biological replicates. Plotted are all S-score pairs among 1052 replicas, consisting of triplicate measurements for 346 histone alleles and wt as well 4 histone alleles measured in duplicates (H4E73Q, H4H18A, H4121A and H4K44Q).

FIG. 7A-7D depict sick and non-viable histone mutant alleles. FIG. 7A: Schematic of the histone point mutants analyzed in this study that were either lethal, could not be constructed after two attempts, or that could not be screened in the pE-MAP (due to e.g. slow growth). Secondary structure elements are indicated as ribbons above the amino acid sequence. The mutation background highlights are color-coded according to the mutation introduced (Table in FIG. 7B), and the mutation font color indicates whether the mutant was lethal (red) or sick (black). Areas with a high incidence of sick and lethal mutations are highlighted (green boxes—nucleosome entry site, yellow boxes—close to dyad axis). FIG. 7B: Table of sick/lethal histone mutants and their hypothesized effects (color-coding as in FIG. 7A). FIG. 7C: Overview of H3 and H4 tail deletion mutants in this study that were either lethal or sick (red: lethal, grey: sick). FIG. 7D: Structural view of sick and lethal point mutants (PDB ID 1ID3 with modeled N-terminal H3 and H4 tails). Histone point mutant alleles, which were attempted but are either lethal or failed construction after multiple attempts and viable mutants too sick for the genetic screen (due to e.g. slow growth) are highlighted in red.

FIG. 8A-8B depict the genetic interaction landscape of histones H3 and H4. FIG. 8A: Hierarchically clustered pE-MAP of 350 histone H3 and H4 alleles screened against a library of 1,370 deletion mutants or hypomorphic alleles (Table 1). The pE-MAP consists of more than 479,000 genetic interactions. Positive-(suppressive/epistatic) and negative (synthetic sick) genetic interactions are colored in yellow or blue, respectively. Examples of histone alleles with similar genetic interaction profiles are highlighted on the right side in context of the nucleosome structure. The nucleosome structure is modified from PDB 1ID3 (Methods), with H3 in purple, H4 in green, and mutated residues highlighted in red. FIG. 8B: Examples of genetic interaction profiles of gene clusters belonging to known protein complexes or biological pathways are highlighted and their genetic interaction profiles enlarged from FIG. 8A. DDR—DNA damage response, UPP—Ubiquitin proteasome pathway.

FIG. 9A-8B depict structural trends of the histone pE-MAP. FIG. 9A: The mean distance between histone mutants belonging to a cluster node of the hierarchically clustered pE-MAP plotted against normalized branch length (red, reference nucleosome PDB: 1ID3). The pE-MAP data was filtered prior to clustering to only contain alleles mapping to residues included in the published nucleosome PDB (number of histone alleles after filtering=222). The mean distance between mutants for 100 randomly generated trees is plotted in black. FIG. 9B: Comparison of the signal of several histone mutant categories as determined in this dataset. Here, signal is defined as the sum of absolute S-scores.

FIG. 10A-10C depict processing of pE-MAP data and calculation of phenotypic similarities. FIG. 10A: To increase the signal-to-noise ratio of the pE-MAP data, the gene deletion profiles were ranked based on the counts of their genetic interaction scores that fell in either the top 2.5% of positive scores or bottom 5% of negative scores of the complete pE-MAP. Gene deletions with the same count were then ranked by the mean of the absolute values of their highest and lowest score. The top 25% of the ranked deletions were retained for computing the point mutant phenotypic profile similarities. FIG. 10B: Comparison of phenotypic profiles using MIC. Pairs of point mutants with more similar profiles (i and j) receive a higher MIC value than those that have less similar profiles (i and k). FIG. 10C: Statistical association of the distance between two mutated residues with their phenotypic similarity. Top: MIC values were computed after ranking gene deletions and selecting top 10, 25, 50, and 100% of the ranked deletions, and plotted against the distance between the two mutated residues in the X-ray structure (PDB: 1I3Q). The grey lines correspond to the upper distance bound used for the implemented distance restraint (Eq. 1). Bottom: Maximum distance for each of the 20 MIC bins. R and p-values correspond to the Pearson correlation coefficient and association significance, respectively, for the log-transformed MIC values.

FIG. 11A-11D depict histone H3-H4 docking results. FIG. 11A: X-ray structure of the histone H3-H4 dimer (PDB: 1ID3). FIG. 11B: Best scoring structure computed by PatchDock.

FIG. 11C: Cα RMSD for the top 100 scoring structures computed by PatchDock. FIG. 11D: Cα RMSD histogram for these structures.

FIG. 12A-12C depict histone MIC value distributions. FIG. 12A: Relationship between the number of protein-wide systematic mutations (often to alanine) in the H3 and H4 subunits and the number of MIC values above the selected MIC value thresholds. Error bars represent one standard deviation from different random sets of mutations in each subunit. The horizontal grey line represents 4 MIC values above the threshold. FIG. 12B: Violin plot showing the MIC value distributions when grouped based on the secondary structure of the residue pairs. FIG. 12C: Violin plot showing the MIC value distributions when grouped based on residue pairs being part of the histone cores or tails.

FIG. 13A-13D depict structural mapping of residue-specific genetic interaction data. FIG. 13A: Average distributions of genetic interaction scores (left) and genetic interaction profile correlations (right) of H3K36 mutants (mean of H3K36A, H3K36R, H3K36Q). Genes required for SET2-mediated H3K36-methylation that exhibit an S-score >2.5 or a genetic interaction profile correlation of at least 0.2 with H3K36 mutants are highlighted. To make the plots comparable, both distributions only include the 1,263 gene deletions/hypomorphs that overlap in the pE-MAP and the correlation map. FIG. 13B: Schematic showing the 4 interactive components depicting the mapping of genetic interactions using the Cytoscape stEMAP app. First, genetic interaction data (here correlations of genetic interaction profiles) and the structure file (modified PDB 1ID3) are imported into Cytoscape creating a residue interaction network (RIN) of the nucleosome and all of the genes that have significant interactions with those residues. The RIN is constructed to reflect the orientation of the 3D view in ChimeraX and the genes are organized to reflect the dendrogram in the original clustered heatmap. The edges are colored to show the interaction (or correlation) scores. Using setsApp, known complexes are loaded to provide a quick way to select the genes in that complex and reflect those genes in all of the windows. The stEMAPP app will display a heatmap showing only the selected genes and their interacting residues and allows the user to adjust various parameters via the controls at the bottom of the panel, including the minimum number of interactions required. In the ChimeraX window the structural view is shown. When a gene or set of genes are selected, the residues that pass the genetic interaction threshold are shown as spheres and colored according to the interaction score (Methods). Selection in the ChimeraX window is linked to the RIN shown in the main window. FIG. 13C: Mapping of genetic interaction profile correlations to SET2 and associated genes, required for H3K36-methylation, on the structure of the nucleosome (modified PDB 1ID3). N-terminal tail residues of H3 and H4 not included in 1ID3 are visualized as strings on the periphery. Only residues that exhibit a median genetic profile correlation >0.2 with the SET2 gene set (SET2, CTK1, EAF3 and RCO1) are highlighted (Methods). H3 (purple), H4 (light green), H2A/H2B and DNA (grey). The red color gradient reflects the strength of the correlation between each residue and the SET2 gene set, calculated as the median correlation between the residue's tested mutations and the SET2 gene set members. FIG. 13D: Mapping of genetic interaction profile correlations of genes required for H3K56-acetylation and deacetylation on the structure of the nucleosome (modified PDB 1ID3). N-terminal tail residues of H3 and H4 not included in 1ID3 are visualized as strings on the periphery. Only alleles that exhibit a median genetic profile correlation >0.2 with RTT109/ASF1 (responsible for H3K56 acetylation, left), or with HST3/HST4 (responsible for H3K56 deacetylation, right) are highlighted (Methods). H3 (purple), H4 (light green), H2A/H2B and DNA (grey). The red color gradient reflects the strength of the correlation between each allele and RTT109/ASF1 or HST3/HST4, calculated as the median correlation between the allele and the respective gene sets.

FIG. 14A-14D depict statistical association of the distance between two mutated residues with their phenotypic similarity for different pE-MAP/CG-MAP datasets. FIG. 14A: Summary of the genetic interaction data used for integrative structure determination for each of the complexes. FIG. 14B: The top panel is the scatter plot of the MIC values and distances between the mutated residues in the WT histone structure (PDB: 1ID3). The bottom plot shows the upper distance bound obtained by binning the MIC values into 20 intervals and selecting the maximum distance spanned by any pair of residues in each bin, followed by fitting a logarithmic decay function to these maximum distances (grey line, Eq. 1). The R-values and p-value are reported for the Pearson correlation between the distances and the log-transformed MIC values (Eq.1). FIG. 14C: Same as FIG. 14B, for subunits Rpb1 and Rpb2 of RNAPII. The grey line is shown for reference and corresponds to the fit to the histone distance upper bound. FIG. 14D: Same as FIG. 14C, for subunits RpoB and RpoC of bacterial RNAP.

FIG. 15 depicts coupling strengths for RpoB and RpoC predicted by RaptorX ComplexContact. Dependance of coupling strengths on the distances between residues in the X-ray structure (PDB: 4YG2) is plotted. Horizontal lines correspond to the coupling strength cutoffs when considering the top L/100, L/50, and L/25 predicted contacts, where L is the length of the concatenated sequence.

FIG. 16A-16D depict estimation of sampling precision for histones H3-H4. FIG. 16A: Convergence of the model scores in the ensemble. Grey dots show that the scores do not continue to improve as more structures are independently computed. The dotted line indicates the highest score in the ensemble. FIG. 16B: Distribution of scores for structures in samples A (dark green) and B (light green), comprising 20,000 random models in the ensemble. The non-parametric Kolmogorov-Smirnov two-sample test (two sided) indicates that the difference between the two score distributions is insignificant (p-value (0.18)>0.05). In addition, the magnitude of the difference is small, as demonstrated by the Kolmogorov-Smirnov two-sample test statistic (D=0.13). FIG. 16C: Three criteria for determining the sampling precision (y-axis), evaluated as a function of the RMSD clustering threshold (x-axis). First, the p-value is computed using the X2-test (one-sided) for homogeneity of proportions (97) (red stars). Second, an effect size for the X2-test is quantified by the Cramer's V value (blue triangles). Third, the population of structures in sufficiently large clusters (containing at least ten structures from each sample) is shown as yellow circles. The vertical dotted grey line indicates the RMSD clustering threshold at which three conditions are satisfied (X2-test p-value (0.18)>0.05 (red, horizontal dotted line), Cramer's V (0.013)<0.10 (blue, horizontal dotted line), and the population of clustered structures (0.995)>0.80 (yellow, horizontal dotted line)), thus defining the sampling precision of 2.05 Å. The three solid curves (in red, blue, and yellow) were drawn through the points to help visualize the results.

FIG. 16D: Population of structures in samples A and B in each of the three clusters obtained by threshold-based clustering using an RMSD threshold of 2.05 Å. The dominant cluster (cluster 1) contains 98% of the structures. Cluster precision and population is shown for each cluster. The precision of the dominant cluster defines the model precision.

FIG. 17A-17D depict RNAPII docking results. FIG. 17A: X-ray structure of Rpb1-Rpb2 (PDB: 1I3Q). FIG. 17B: Best scoring docking structure computed by PatchDock. FIG. 17C: Cα RMSD for the top 100 scoring models obtained using rigid body docking structures computed by PatchDock. FIG. 17D: Cα RMSD histogram for these top 100 scoring models obtained using rigid body docking.

FIG. 18A-18B depict histone pE-MAP quality and signal. FIG. 18A: ROC curves showing the power to predict physical interactions between pairs of proteins from this pE-MAP (blue) as well as previously published pE-MAP (green, (75)) and E-MAP (black, (76)) data. FIG. 18B: Relationship between gene expression (log 2-fold change) and genetic interaction profiles (S-scores) of 29 H3 and H4 alleles (Table 6). Data from all 1,256 deletion library mutants that were measured in both RNAseq expression and pE-MAP analysis are plotted.

FIG. 19A-19I depict genetic interactions connect H3 and H4 residues to cellular pathways. FIG. 19A: Heatmap representation of gene set enrichment analysis (GSEA, Methods) to unveil functional connections between histone residues and biological processes related to nuclear function. Genetic interaction profiles of 350 histone alleles were correlated to 4414 genetic interaction profiles from previous studies. The resulting matrix of 350×4414 correlation coefficients (CCs) was used for GSEA. Only modifiable histone residues are included in the heatmap, and the color indicates the False Discovery Rate (FDR) between each residue and biological process. Residues, processes and connections that are further detailed in FIG. 19B-19I are highlighted. FIG. 19B: Residues connected to DNA recombination & repair at FDR<10-6 were ranked by mean correlation, and K->R and R->K mutations of the top 3 residues were examined for their effect on mutation frequency. Spontaneous mutation frequency was measured by a 5-FOA resistance assay at the URA3 locus. Error bars indicate standard error of the mean (SEM). FIG. 19C: Effect on K56ac-levels of R->K histone mutants that were found to alter mutation frequency, measured by quantitative mass spectrometry. Rtt109 is required for H3K56 acetylation and its deletion serves as a positive control (135). Error bars indicate SEM. FIG. 19D: Workflow for cryptic transcription assay. Mutants predicted by GSEA to be involved in cryptic transcription (FDR<10⁻⁶) were assayed for 5′- and 3′-transcript abundance by qPCR at the STE11 gene. A greater output of transcripts from the 3′ region than the 5′ region indicates the presence of transcription start sites within the gene that give rise to cryptic transcripts. FIG. 19E: 5′ (red) and 3′ (blue) transcript abundance changes in histone mutants compared to wt (fold-change) at the STE11 gene. set2Δ is shown as a positive control, followed by the mutants predicted to exhibit cryptic transcription (FDR<10⁻⁶) that result in over two-fold change of 3′ transcript abundance (H3K36A to H3K122Q), and finally three mutants not predicted to exhibit cryptic transcription (H4S64D, H3T6D and H3K121R). All measured replicates are shown and the bar heights indicate the geometric means. FIG. 19F: 5′ (red) and 3′ (blue) transcript abundance changes in mutants compared to wt (fold-change) at the STE11 gene. Measured S-scores of double mutants are displayed above the labels. All measured replicates are shown and the bar heights indicate the geometric means. FIG. 19G: ATAC-seq workflow. ATAC-seq is used to determine nucleosome-free regions on a genome-wide scale. FIG. 19H: ATAC-seq reads from open chromatin regions in the gene body of STE11 (blue). Nucleosome free regions (NFRs) overlapping with previously reported start sites of cryptic transcripts and sequences important for transcription initiation are highlighted (NFR—red boxes, *—TATA-box element, BRE—B response element). FIG. 19I: Gene body plots of open chromatin regions within 11 genes (FLO8, AVO1, LCB5, SMC3, SPB4, APM2, DDC1, SYF1, OMS1, PUS4, STE11) known to give rise to cryptic transcripts. The plots indicate the difference between each mutant and WT and the dashed lines at 0 indicate the WT chromatin accessibility. The gene lengths were normalized by binning all genes to a set number of intervals and plotting the average normalized read count for all the base pairs in each interval (Methods).

FIG. 20A-20B depict effect of histone alleles on SET2-mediated H3K36 methylation. FIG. 20A: H3K36-trimethylation (H3K36me3) levels as determined by Western Blot of H3K36A, H3K122A and H3K122Q compared to wildtype H3K36me3-levels. Histone H3 was used as loading control. L—long exposure, S—short exposure. FIG. 20B: 5′ (red) and 3′ (blue) transcript abundance changes in mutants compared to wt (fold-change) at the STE11 gene. All measured replicates are shown and the bar heights indicate the geometric means.

FIG. 21A-21C depict the methodology by which MIC is calculated. FIG. 21A: For each pair (x,y), the MIC algorithm finds the x-by-y grid with the highest induced mutual information. FIG. 21B: The algorithm normalizes the mutual information scores and compiles a matrix that stores, for each resolution, the best grid at that resolution and its normalized score. FIG. 21C: The normalized scores form the characteristic matrix, which can be visualized as a surface; MIC corresponds to the highest point on this surface. In this example, there are many grids that achieve the highest score. The star in FIG. 21B marks a sample grid achieving this score, and the star in FIG. 21C marks that grid's corresponding location on the surface.

### DETAILED DESCRIPTION OF EMBODIMENTS

Before the present systems and methods are described, it is to be understood that the present disclosure is not limited to the particular processes, compositions, or methodologies described, as these may vary. It is also to be understood that the terminology used in the description is for the purposes of describing the particular versions or embodiments only, and is not intended to limit the scope of the present disclosure. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the methods, devices, and materials in some embodiments are now described. All publications mentioned herein are incorporated by reference in their entirety. Nothing herein is to be construed as an admission that the present disclosure is not entitled to antedate such disclosure by virtue of prior invention.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, +0.8%, ±0.7%, +0.6%, +0.5%, ±0.4%, +0.3%, ±0.2% or +0.1% from the specified value as such variations are appropriate to perform the disclosed methods. When “about” is present before a series of numbers or a range, it is understood that “about” can modify each of the numbers in the series or range.

The term “at least” prior to a number or series of numbers (e.g. “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

Ranges provided herein are understood to include all individual integer values and all subranges within the ranges.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The term “diagnosis” or “prognosis” as used herein refers to the use of information (e.g., genetic information or data from other molecular tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

As used herein, the phrase “in need thereof” means that the animal or mammal has been identified or suspected as having a need for the particular method or treatment. In some embodiments, the identification can be by any means of diagnosis or observation. In any of the methods and treatments described herein, the animal or mammal can be in need thereof.

The term “interaction” as used herein refers to a reciprocal action between a first protein and a second protein.

As used herein, the term “mammal” means any animal in the class Mammalia such as rodent (i.e., mouse, rat, or guinea pig), monkey, cat, dog, cow, horse, pig, or human. In some embodiments, the mammal is a human. In some embodiments, the mammal refers to any non-human mammal. The present disclosure relates to any of the methods or compositions of matter wherein the sample is taken from a mammal or non-human mammal. The present disclosure relates to any of the methods or compositions of matter wherein the sample is taken from a human or non-human primate.

As used herein, the term “predicting” refers to making a finding that a first protein has a significantly enhanced probability or likelihood to interact with a second protein.

A “score” is a numerical value that may be assigned or generated after normalization of the value based upon the presence, absence, or quantity of deposition of amyloid-beta (Aβ) protein and tau protein in the brain of a subject. In some embodiments, the score is normalized in respect to a control data value.

As used herein, the term “stratifying” refers to sorting individuals into different classes or strata based on the features of a neurological disease. For example, stratifying a population of individuals with Alzheimer's disease involves assigning the individuals on the basis of the severity of the disease (e.g., mild, moderate, advanced, etc.).

As used herein, the term “subject,” “individual” or “patient,” used interchangeably, means any animal, including mammals, such as mice, rats, other rodents, rabbits, dogs, cats, swine, cattle, sheep, horses, or primates, such as humans. In some embodiments, the subject is a human having a disorder. In some embodiments, the subject is a human having cancer. In some embodiments, the subject is a healthy human being.

As used herein, the term “threshold” refers to a defined value by which a normalized score can be categorized. By comparing to a preset threshold, a subject, with corresponding qualitative and/or quantitative data corresponding to a normalized score, can be classified based upon whether it is above or below the preset threshold.

As used herein, the terms “treat,” “treated,” or “treating” can refer to therapeutic treatment and/or prophylactic or preventative measures wherein the object is to prevent or slow down (lessen) an undesired physiological condition, disorder or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent or improve an unwanted condition or disease of a patient.

A “therapeutically effective amount” or “effective amount” of a composition is a predetermined amount calculated to achieve the desired effect, i.e., to treat, combat, ameliorate, prevent or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the particular circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds of embodiments of the present disclosure is typically an amount such that when it is administered in a physiologically tolerable excipient composition, it is sufficient to achieve an effective systemic concentration or local concentration in the tissue.

The terms “phenotypic profile” as used herein means a profile of genetic mutations associated with a particular phenotype displayed by a modeled protein-protein interaction. In some embodiments, the protein-protein interaction is modeled by calculating an S-score or series of S-scores that correspond to a first and second nucleic acid, or a first nucleic acid sequence and its spatial position in a genome of a cell relative to the spatial position of one or a plurality of other nucleic acids in the genome. In some embodiments, the phenotypic interaction profile is a genetic interaction profile, such as pE-MAP. In some embodiments, the phentotypic interaction profile is a chemical-genetic interaction profile, such as CG-MAP. In some embodiments, the generation of a phenotypic profile assigned to the spatial relationship assigned to nucleic acids can be used to predict the structure of the interaction between the amino acid encoded by the nucleic acids. In such methods and in some embodiments, the methods comprise calculating a MIC or calculating a Pearson coefficient, such MIC or Pearson coefficient corresponding to a position of any encoded amino acid in a virtual display of the modeled protein-protein interaction.

The terms “chemical-genetic interaction profile” as used herein mean a type of phenotypic profile comprising a collection of chemical-genetic interactions scores for a given nucleic acid. In some embodiments, the scores are S scores as determined by the computational analysis provided herein.

The terms “genetic interaction phenotypic profile” means a type of phenotypic profile comprising a collection of genetic interactions (S-scores) for a given nucleic acid. In some embodiments the results of calculating those S-Scores is used to generate a pE-MAP of a given nucleic acid. In some embodiments, the genetic interaction phenotype profile is obtained by performing any one or more of the following: epistatic miniarray profile (E-MAP), synthetic genetic array (SGA) and diploid synthetic lethal analysis by microarray (dSLAM).

The terms “mapping” as used herein means a systematic measurement of biological readouts for a number of mutations in at least two different components, followed by computational analysis and scoring. In some embodiments, the two components of the computation analysis are a first and second nucleic acid sequence within a genome of a pathogen, cell or subject (e.g. a genetic interaction). In some embodiments, the wherein the pE-MAP or the CG-MAP is derived from genetic interaction (pE-MAP) or chemical-genetic interactions (CG-MAP) of nucleic acid carried on a vector in an vitro assay. In some embodiments, the disclosed methods include a step of obtaining the genetic sequence of or sequencing all or a portion of the genome of a cell, pathogen or subject in an in vitro assay prior to mapping or selecting one or a plurality of nucleic acids with which to perform the mapping. In some embodiment, the disclosed methods include a step of creating a point mutation within the isolated genome of a cell, isolated DNA from a subject, or isolated nucleic acid of a pathogen and selecting the mutated nucleic acid as one of the nucleic acids to be mapped prior to the step of mapping.

The terms “designing spatial restraints” as used herein means structure modeling is done based on restraints between how far two amino acid residue are allowed to be from each other.

The terms “integrative structure modeling” as used herein means a computational approach that combines different types of biological measurements or data to generate models of protein (or RNA) structures.

Methods of Creating a Model and Identifying Protein-Protein Interactions

The disclosure relates to a method of creating a model of a protein-protein interaction comprising a step of mapping the spatial relationship of a first nucleic acid between one or a plurality of other nucleic acids. In some embodiments, the step of mapping comprises calculation of one or a plurality of S-scores that correspond to a chosen nucleic acid or pair of nucleic acids or series of nucleic acids. In some embodiments, the mapping comprises the step of creating a chemical-genetic interaction profile and/or genetic interaction profile by calculating one or a plurality of S-scores that correspond to a chosen nucleic acid or pair of nucleic acids or series of nucleic acids. In some embodiments, after creating a chemical-genetic interaction profile and/or genetic interaction profile, the method comprises calculating a MIC or Pearson coefficient relative to the spatial position of the amino acid sequence encoded by the nucleic acid sequences. In some embodiments, after the MIC and/or Pearson coefficients are calculated, the method comprises generating a model of protein-protein interactions as between the amino acid sequences based upon any or all of the above calculations. In some embodiments, the methods comprise displaying an image on a display of a system disclosed herein, such image corresponding to the model of the protein-protein interaction. The instant paragraph is a non-limiting example of an embodiment of analysis performed in generated a model of a protein-protein interaction.

In some embodiments, the disclosure relates to the method or step of modeling in the disclosed steps comprising the step of mapping a protein-protein interaction between a first and second amino acid sequence corresponding to and encoded by a first nucleic acid and a second nucleic acid sequence, respectively. In some embodiments, the disclosure relates to the method or step of modeling in the disclosed steps comprising the step of mapping a protein-protein interaction between a first and second amino acid sequence corresponding to and encoded by a first nucleic acid and a series of nucleic acid sequences. In some embodiments, the step of mapping is performed after a step of selecting a first nucleic acid sequence, a second nucleic acid sequence, and, optionally, a third nucleic acid sequence. In some embodiments, the method of modeling, method of selecting or characterizing a target associated with a disorder, or selecting characterizing a protein-protein interaction as being associated with a disorder comprises a first step of selecting a step of selecting a first nucleic acid sequence, a second nucleic acid sequence, and, optionally, a third nucleic acid sequence. In some embodiments, the step of selecting the first nucleic acid sequence, second nucleic acid sequence or third nucleic acid sequences are chosen by scanning, sequencing, or obtaining the sequence of a genome of a pathogen, a cell in vitro or a subject, such as a human subject. In some embodiments, the pathogen is a virus, bacteria or parasite or unicellular organism. In some embodiments, the methods comprise a step of selecting a nucleic acid sequence from a subject, cell or pathogen, mutating the nucleic acid sequence and performing a modeling analysis disclosed herein to create a model of a protein-protein interaction model associated with the mutated nucleic acid. In some embodiments, the methods further comprises a step of repeating the modeling analysis step by choosing the a wild-type or unmutated nucleic acid sequence to create a model of protein-protein interaction model associated with the wild-type of unmutated nucleic acid sequence. In some embodiments, the methods further comprise a step of comparing the model associated with the nucleic acid comprising a mutation to the model associated with the nucleic acid free of the mutation.

The disclosure also relates to a method of identifying an effect of a mutation or perturbation in a protein-protein interaction comprising: (a) mutating one or more nucleic acids in the genome of a cell; (b) analyzing the mutation by devising a phenotypic profile associated with the mutation by modeling changes or effect in protein-protein interactions by the presence of mutation in other nucleic acids or exposure to chemicals; (c) comparing the phenotypic profile associated with the mutation with the phenotypic profile associated with one or more nucleic acids carrying other mutations or free of mutation (wild-type). In some embodiments, the cell is a cell in tissue culture. In some embodiments, the methods further comprise the step (d) classifying a phenotype as being associated with the mutation based the comparison of phenotypic profiles.

The disclosure relates to a method of characterizing or selecting a target for an agent comprising (a) determining a model of protein-protein interaction; comparing the model of the protein-protein interaction with the known mechanism of action and/or model of agent interaction at, on or proximate to one of the amino acid sequences being modeled; and (c) characterizing or selecting an agent that could modulate the binding or association of the protein-protein interaction based upon the agent's position in the model. In some embodiments, for example, if the agent (such as a small molecule or chemical agent) is known to bind one or both of the amino acid sequences that is modeled by step (a) at one or several amino acid positions with the amino acid sequence thereby preventing the protein-protein interaction, the agent could be characterized as a potential lead compound for treatment of a disorder associated with a phenotype caused by the protein-protein interaction. Similarly, in the above example, the protein-protein interaction could be selected or characterized as an agent or therapeutic target. In some embodiments, if the agent (such as a small molecule or chemical agent) is known to bind one or both of the amino acid sequences that is modeled by step (a) at one or several amino acid positions with the amino acid sequence thereby strengthening or encouraging the protein-protein interaction, the agent could be characterized as a potential lead compound for treatment of a disorder associated with a phenotype caused by the absence of the protein-protein interaction. Similarly, in the above embodiment, the protein-protein interaction could also be selected or characterized as an agent target or therapeutic target.

Therefore the disclosure relates to a method of characterizing, identifying or selecting a protein-protein interaction as a target for therapy of a disorder comprising: (a) determining a model of a protein-protein interaction; (b) comparing the model protein-protein interaction with a known mechanism of action of an agent or agent interaction associated with the protein-protein interaction; selecting the protein-protein interaction as a target for a disorder if the protein-protein interaction would be modulated by the mechanism of action of an agent or agent interaction associated with the protein-protein interaction. In some embodiments, the agent is a small molecule that binds or associates with an amino acid sequence at least one of the amino acid sequences that are a part of the protein-protein interaction. In some embodiments, the method of determining a model of protein-protein interaction comprises mapping the spatial relationship of a first nucleic acid between one or a plurality of other nucleic acids. In some embodiments, the step (a) comprises mapping comprises calculation of one or a plurality of S-scores that correspond to a chosen nucleic acid or pair of nucleic acids or series of nucleic acids. In some embodiments, the method of characterizing or selecting a target comprises a step of mapping, such mapping comprises the step of creating a chemical-genetic interaction profile and/or genetic interaction profile by calculating one or a plurality of S-scores that correspond to a chosen nucleic acid or pair of nucleic acids or series of nucleic acids. In some embodiments, after creating a chemical-genetic interaction profile and/or genetic interaction profile, the method comprises calculating a MIC or Pearson coefficient relative to the spatial position of the amino acid sequence encoded by the nucleic acid sequences. In some embodiments, after the MIC and/or Pearson coefficients are calculated, the method comprises generating a model of protein-protein interactions as between the amino acid sequences based upon any or all of the above calculations. In some embodiments, the methods comprise displaying an image on a display of a system disclosed herein, such image corresponding to the model of the protein-protein interaction. The instant paragraph is a non-limiting example of an embodiment of analysis performed in generated a model of a protein-protein interaction.

The disclosure relates to a method of identifying/prioritizing drugs that target a protein-protein interaction; comprising the steps outlined above to determine the structure of a protein-protein interaction; and then using the interface structure determined by the disclosed computation analyses as a target for computational docking of small molecule drugs or biologics to bind to the interaction interface.

The disclosure also relates to a method of identifying/prioritizing drugs that target a protein-protein interaction; comprising the steps outlined above to determine the structure of a protein-protein interaction, and then repeating the process in the presence of different compounds to identify those that affect the structure of the protein-protein interaction.

Any of the protein-protein interaction modeling can be performed by isolating, sequencing and mutating one or more nucleic acid sequences from the genome of cell or pathogen in vitro. Subsequently, the methods can comprise a step of generating a phenotypic profile of the mutation and the same nucleic acid free of the mutation and then comparing those profiles to visualize how the mutation changes the model of the protein-protein interaction. In some embodiments, by comparing a known interaction model of a drug, biologic or other agent to the protein-protein interaction modeled and associated to the mutation, methods further comprise a step screening for or selecting a therapeutic with a likelihood of successfully treating a disorder associated with the mutation. In some embodiments, the methods can be performed on a system comprising the computer program product disclosed herein. In some embodiments, the methods further comprise the step of performing a functional bioassay to validate the predictive nature of the model.

The disclosure relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic mini-array profile (pE-MAP) or a chemical genetics mini-array profile (CG-MAP) associated with the first nucleic acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

The disclosure relates to a method of determining structure of a protein-protein interaction, said method comprising: (a) selecting a first nucleic acid sequence and a second nucleic acid sequence, wherein the second nucleic acid sequence is a wild-type sequence relative to the first nucleic acid sequence, and wherein at least the first nucleic acid sequence comprises a point mutation relative to the second nucleic acid sequence; (b) creating a point-mutant epistatic mini-array profile (pE-MAP) or a chemical genetics mini-array profile (CG-MAP) associated with the first nucleic acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

The disclosure further provides a method of modeling a three-dimensional structure as between two or more amino acid sequences, said method comprising: (a) selecting a first amino acid sequence and a second amino acid sequence, wherein the second amino acid sequence is a wild-type sequence relative to the first amino acid sequence, and wherein at least the first amino acid sequence comprises a point mutation relative to the second amino acid sequence; (b) creating a point-mutant epistatic mini-array profile (pE-MAP) or a chemical genetics mini-array profile (CG-MAP) associated with the first amino acid sequence; (c) calculating an S-score associated with the first nucleic acid sequence and the third nucleic acid sequence; (d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject; (e) correlating the MIC and the pE-MAP, or the MIC and the CG-MAP, with: (i) spatial positions of amino acid residues within the first amino acid sequence; and (ii) spatial positions of amino acid residues within the third amino acid sequence; and (f) mapping a protein-protein interaction as between the first amino acid sequence and the third amino acid sequence.

In some embodiments, the correlating step of any of the disclosed methods comprises calculating a Pearson correlation. In some embodiments, the correlating step of any of the disclosed methods comprises: i) calculating a plurality of MICs; ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; iii) performing a noise model for calculation of a standard deviation; and iv) formulating spatial restraints as Bayesian data likelihoods. In some embodiments, the upper distance bound is calculated by: a) binning the plurality of MICs into 20 intervals; b) selecting a maximum distance spanned by any pair of amino acid residues in each bin; and c) fitting a logarithmic decay function (dᵤ) to the upper distance bound:

$${d}_{U} ( M I C ) = \{ \begin{matrix}\frac{log ( M I C ) - n}{k} & if M I C \leq 0.6 \\ 20 & if M I C > 0.6\end{matrix} \qquad ( 1 )$$

wherein n is −0.0147 and k is −0.41.

In some embodiments, the spatial restraints are formulated as Bayesian data likelihoods by calculating a posterior probability of model M given data D and prior information I by:

p(M|D,I)∝p(D|M,I)·p(M|I)

wherein the model, M, consists of a structure X and unknown parameters Y, prior p(M|I) is the probability density of model M given I, and likelihood function p(D|M, I) is the probability density of observing data D given M and I.

In some embodiments, the disclosed method further comprises calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein f_{i,j}(X) is a forward model that predicts the data point d_{i,j} in D that would have been observed for structure Xin an experiment without noise, and is defined as:

$${f}_{i , j} ( X ) = M I C ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

wherein d₀=dᵤ(0.6); wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:

$$P ( M I {C}_{i , j}^{obs} \vert M I C i , j , X , {σ}_{i , j} ) = \{ \begin{matrix}\frac{1}{N} & if M I {C}_{i , j}^{obs} \geq M I {C}_{i , j} \\ \frac{1}{M} \frac{1}{\sqrt{2 {πσ}_{i , j}^{2}} M I {C}_{i , j}^{obs}} exp ( - \frac{1}{2 {σ}_{i , j}^{2}} {log}^{2} ( \frac{M I {C}_{i , j}^{obs}}{M I {C}_{i , j}} ) ) & if M I {C}_{i , j}^{obs} < M I {C}_{i , j}\end{matrix} \qquad ( 3 )$$

wherein σ_{i,j} are the noise parameters that can optionally be determined as part of the model, and N and Mare normalization factors necessary to make the likelihood continuous.

In some embodiments, the disclosed method further comprises obtaining a Bayesian term in the scoring function defining as the negative logarithm of the posterior probability density:

S(M)=−log p(M|D,I).

In some embodiments, the mapping of step (f) comprises performing a structure E-MAP (stE-MAP) as between expression of the first nucleic acid sequence and the third nucleic acid sequence. In some embodiments, the mapping of the protein-protein interaction has a resolution from about 1 to about 10 angstroms. In some embodiments, the mapping of the protein-protein interaction has a resolution from about 2 to about 9 angstroms. In some embodiments, the mapping of the protein-protein interaction has a resolution from about 3 to about 8 angstroms. In some embodiments, the mapping of the protein-protein interaction has a resolution from about 4 to about 7 angstroms. In some embodiments, the mapping of the protein-protein interaction has a resolution from about 5 to about 6 angstroms. In some embodiments, the mapping of the protein-protein interaction has a resolution of about 1 angstroms. In some embodiments, the mapping of the protein-protein interaction has a resolution of about 2 angstroms. In some embodiments, the mapping of the protein-protein interaction has a resolution of about 3 angstroms. In some embodiments, the mapping of the protein-protein interaction has a resolution of about 4 angstroms. In some embodiments, the mapping of the protein-protein interaction has a resolution of about 5 angstroms. In some embodiments, the mapping of the protein-protein interaction has a resolution of about 6 angstroms. In some embodiments, the mapping of the protein-protein interaction has a resolution of about 7 angstroms. In some embodiments, the mapping of the protein-protein interaction has a resolution of about 8 angstroms. In some embodiments, the mapping of the protein-protein interaction has a resolution of about 9 angstroms. In some embodiments, the mapping of the protein-protein interaction has a resolution of about 10 angstroms. In some embodiments, the mapping of the protein-protein interaction has a resolution of about 11 angstroms. In some embodiments, the mapping of the protein-protein interaction has a resolution of about 12 angstroms. In some embodiments, the mapping of the protein-protein interaction has a resolution of about 13 angstroms. In some embodiments, the mapping of the protein-protein interaction has a resolution of about 14 angstroms. In some embodiments, the mapping of the protein-protein interaction has a resolution of about 15 angstroms.

The S-score for the disclosed method can be determined by the following: double deletions were scored as to the magnitude and sign of the observed genetic interaction. We wanted a score that would reflect both our confidence in the presence of genetic interactions as well as the strengths of interactions, and so we chose to use a modified t-value score (S). A standard t-value is computed as:

t=(μ_{Exp}−μ_{Cont})/Sqrt(s_{Var}/n_{Exp}+s_{Var}/n_{Cont})

where:

s_{Var}=(var_{Exp}×(n_{Exp}−1)+var_{Cont}×(n_{Cont}−1))/(n_{Exp}+n_{Cont}−2)

where: μ_{Exp}=mean of normalized colony sizes for the double mutant of interest; var_{Exp}=the variance of the normalized colony sizes for the double mutant of interest; n_{Exp}=number of measurements of colony sizes for the double mutant (typically, this value was 6, although it differed slightly (4, 10, and so on) for a small number of double mutants); μ_{Cont}=mean of normalized colony sizes for the control KAN-marked single mutant strain corresponding to the double mutant of interest; var_{Cont}=the variance of the normalized colony sizes for this control KAN-marked strain; and n_{Cont}=the number of measurements of colony sizes for this control KAN-marked strain.

The S score is constructed in the same way:

S=(μ_{Exp}−μ_{Cont})/sqrt(s_{Var}/n_{Exp}+s_{Var}/n_{Cont})

where:

s_{Var}=(var_{Exp}×(n_{Exp}−1)+var_{Cont}×(n_{Cont}−1))/(n_{Exp}+n_{Cont}−2)

but with the following modifications: cont=median of normalized colony sizes for all double mutants containing the KAN-marked mutant of interest; var_{Exp}=the maximum of the variance of normalized colony sizes for the double mutant of interest or a minimum bound described below; var_{Cont}=median of the variances in normalized colony sizes observed for all double mutants containing the KAN-marked mutant of interest or a minimum bound described below; and n_{Cont}=6 (this was the median number of experimental replicates over all the experiments).

A minimum bound was placed on the experimental standard deviation (and hence on the variance) because we observed that occasionally, by chance, six repeated measurements would give an unusually small standard deviation, resulting in a large score, but these large scores did not seem to be reproducible, nor did they reflect strong genetic interactions. We therefore placed a minimum bound on this standard deviation equal to the expected standard deviation in normalized colony size for a double mutant made from NAT- and KAN-marked mutants with similar growth phenotypes. The expected standard deviation was calculated by measuring the observed standard errors in measurement as a function of both unnormalized colony size typical of the NAT-marked mutant and as a function of normalized colony size typical of the KAN-marked mutant.

For similar reasons as for var_{Exp} and because it improved the reproducibility of computed S scores, a lower bound was also placed on var_{Cont}. This lower bound was equal to cont multiplied by the observed median relative error (standard deviation divided by mean size) for all measurements in the data set.

Both of these measures may be biased if the frequency of synthetic interactions is significantly greater or smaller than the frequency of alleviating interactions for a particular gene. However, we have observed this bias to be relatively small (data not shown), and we include in our MATLAB toolbox an alternative strategy to estimate the typical colony size on an experimental plate or for a given KAN-marked mutant. The alternative strategy, which uses a Parzen Window approach to estimate the most common colony size, is less sensitive to skewed distributions of colony sizes.

The MIC can be obtained by the following: Intuitively, MIC is based on the idea that if a relationship exists between two variables, then a grid can be drawn on the scatterplot of the two variables that partitions the data to encapsulate that relationship. Thus, to calculate the MIC of a set of two-variable data we explore all grids up to a maximal grid resolution, dependent on the sample size, computing for every pair of integers (x,y) the largest possible mutual information achievable by any x-by-y grid applied to the data. We then normalize these mutual information values to ensure a fair comparison between grids of different dimensions, and to obtain modified values between zero and one. We define the characteristic matrix M=(m_{x,y}), where m_{x,y} is the highest normalized mutual information achieved by any x-by-y grid, and the statistic MIC to be the maximum value in M.

More formally, for a grid G, let I_{G} denote the mutual information of the probability distribution induced on the boxes of G, where the probability of a box is proportional to the number of data points falling inside the box. The (x,y)-th entry m_{x,y} of the characteristic matrix equals max{I_{G}}/log min{x,y}, where the maximum is taken over all x-by-y grids G. MIC is the maximum of m_{x,y} over ordered pairs (x,y) such that xy<B, where B is a function of sample size; we usually set B=n^{0.6}.

Every entry of M falls between zero and one, and so MIC does as well. MIC is also symmetric (i.e. MIC(X, Y)=MIC(Y, X)) due to the symmetry of mutual information, and because I_{G} depends only on the rank order of the data, MIC is invariant under order-preserving transformations of the axes. Importantly, although mutual information is used to quantify the performance of each grid, MIC is not an estimate of mutual information.

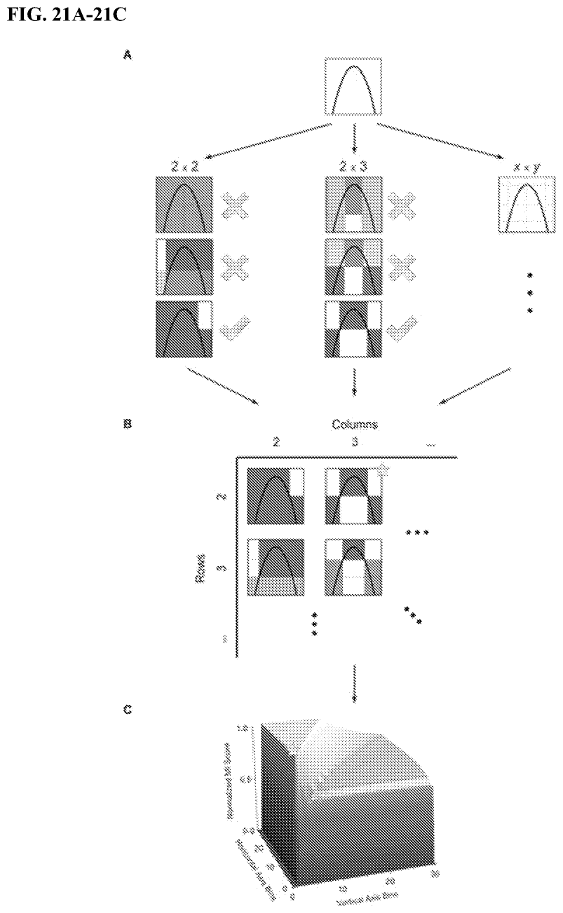

To calculate M, we would ideally optimize over all possible grids. For computational efficiency, we instead use a dynamic programming algorithm that optimizes over a subset of the possible grids and appears to approximate well the true value of MIC in practice. The methodology by which a MIC is calculated is depicted in FIG. 21A-21C.

In some embodiments, the disclosed methods further comprise employing one or more traditional structural biology methods, such as X-ray crystallography, nuclear magnetic resonance (NMR) spectroscopy, and electron microscopy (EM), such as cryogenic electron microscopy (cryo-EM), sequentially with any of the disclosed methods to validate the prediction.

In some embodiments, the disclosure relates to a method of identifying perturbations in a protein-protein interaction comprising: (a) mutating one or more nucleic acids in the genome of a cell; (b) analyzing the mutation by devising a phenotypic profile associated with the mutation; wherein the step of analyzing comprises comparing the phenotypic profile associated with the mutation with the phenotypic profile associated with one or more nucleic acids free of the mutation.

In some embodiments, the disclosure relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic miniarray profile (pE-MAP) and/or a chemical genetics miniarray profile (CG-MAP) associated with the first nucleic acid sequence by calculating an S-score associated with the first nucleic acid sequence and a third nucleic acid sequence; (d) calculating a maximal information coefficient (MIC) corresponding to the first nucleic acid sequence; (e) correlating the MIC with: (i) spatial positions of amino acid residues within an amino acid sequence encoded by the first nucleic acid sequence; and (ii) spatial positions of amino acid residues within an amino acid sequence encoded by the third nucleic acid sequence; and (f) mapping a protein-protein interaction as between the amino acid sequence encoded by the first nucleic acid sequence and the amino acid sequence encoded by the third nucleic acid sequence.

In some embodiments, the methods further comprise a step of displaying an image of the protein-protein interaction on a display as a component of any of the disclosed systems after the step of mapping is completed.

In some embodiments, the disclosure also relates to a method of identifying a protein-protein interaction associated with a disorder, said method comprising: (a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence; (b) creating a point-mutant epistatic miniarray profile (pE-MAP) and/or a chemical genetics miniarray profile (CG-MAP) associated with the first nucleic acid sequence by calculating an S-score associated with the first nucleic acid sequence and a third nucleic acid sequence; (c) calculating a maximal information coefficient (MIC) between the pE-MAP or CG-MAP of the first nucleic acid sequence and the third nucleic acid sequence; (d) correlating the MIC with: (i) spatial positions of amino acid residues within an amino acid sequence encoded by the first nucleic acid sequence; and (ii) spatial positions of amino acid residues within an amino acid sequence encoded by the third nucleic acid sequence; and (e) mapping a protein-protein interaction as between the amino acid sequence encoded by the first nucleic acid sequence and the amino acid sequence encoded by the third nucleic acid sequence.

In some embodiments, the methods further comprise a step of displaying an image of the protein-protein interaction on a display as a component of any of the disclosed systems after the step of mapping is completed.

In some embodiments, the disclosure also relates to a method of determining structure of a protein-protein interaction, said method comprising: (a) selecting a first nucleic acid sequence and a second nucleic acid sequence, wherein the second nucleic acid sequence is a wild-type sequence relative to the first nucleic acid sequence, and wherein at least the first nucleic acid sequence comprises a point mutation relative to the second nucleic acid sequence; (b) creating a point-mutant epistatic miniarray profile (pE-MAP) or a chemical genetics miniarray profile (CG-MAP) associated with the first nucleic acid sequence; (c) calculating a maximal information coefficient (MIC) between the pE-MAP or CG-MAP of the first nucleic acid sequence and the third nucleic acid sequence; (d) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and (e) mapping a protein-protein interaction as between the amino acid sequence encoded by the first nucleic acid sequence and the amino acid sequence encoded by the third nucleic acid sequence.

In some embodiments, the disclosure also relates to a method of modeling a three-dimensional structure as between two or more amino acid sequences, said method comprising: (a) selecting a first amino acid sequence and a second amino acid sequence, wherein the second amino acid sequence is a wild-type sequence relative to the first amino acid sequence, and wherein at least the first amino acid sequence comprises a point mutation relative to the second amino acid sequence; (b) creating a point-mutant epistatic miniarray profile (pE-MAP) or a chemical genetics miniarray profile (CG-MAP) associated with the first nucleic acid sequence; (c) calculating a maximal information coefficient (MIC) between the pE-MAP or CG-MAP of the first nucleic acid sequence and the third nucleic acid sequence; (d) correlating the MIC and the pE-MAP, or the MIC and the CG-MAP, with: (i) spatial positions of amino acid residues within the first amino acid sequence; and (ii) spatial positions of amino acid residues within the third amino acid sequence; and (e) mapping a protein-protein interaction as between the first amino acid sequence and the third amino acid sequence.

In some embodiments, the disclosure also relates to a computer program product encoded on a computer-readable storage medium, wherein the computer program product comprises instructions for: (a) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence; (b) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and (c) mapping a protein-protein interaction as between the amino acid sequence encoded by the first nucleic acid sequence and the amino acid sequence encoded by the third nucleic acid sequence.

In some embodiments, the disclosure also relates to a computer program product encoded on a computer-readable storage medium, wherein the computer program product comprises instructions for: (a) generating a pE-MAP or CG-MAP by calculating S-scores associated with a first nucleic acid sequence wherein the first nucleic acid sequence comprises a point mutation relative to a second sequence which is a wild-type sequence relative to the first nucleic acid sequence; (b) calculating a maximal information coefficient (MIC) between the pE-MAP or CG-MAP of the first nucleic acid sequence and a third nucleic acid sequence.

In some embodiments, the step of correlating comprises: (i) calculating a plurality of MICs as between the first sequence and a series of nucleic acid sequences comprising the third nucleic acid sequence; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In some embodiments, the disclosure also relates to a system for identifying a protein interaction network in a subject, the system comprising: (a) a processor operable to execute programs; (b) a memory associated with the processor; (c) a database associated with said processor and said memory; and (d) a program stored in the memory and executable by the processor, the program being operably for: (i) calculating a maximal information coefficient (MIC) associated with a first nucleic acid sequence and a second nucleic acid sequence, wherein the first nucleic acid sequence comprises a point mutation relative to a second sequence which is a wild-type sequence relative to the first nucleic acid sequence; (ii) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the second nucleic acid sequence; and (iii) mapping a protein-protein interaction as between the amino acid sequence encoded by the first nucleic acid sequence and the amino acid sequence encoded by the second nucleic acid sequence.

In some embodiments, the method of modeling a protein-protein interaction, said method comprising: (a) calculating a plurality of MICs associated with a first nucleic acid sequence relative to a plurality of nucleic acid sequences free of the first nucleic acid sequence, wherein the first nucleic acid sequence comprises a point mutation relative to a known sequence; (b) calculating an upper distance bound between the spatial positions of the amino acid residues within the amino acid sequence encoded by the first nucleic acid sequence and the spatial positions of the amino acid residues within the amino acid sequence encoded by the plurality of nucleic acid sequences free of the first nucleic acid sequence; (c) performing a noise model for calculation of a standard deviation; and (d) formulating spatial restraints as Bayesian data likelihoods.

In some embodiments, the disclosure also relates to a method of creating a genetic interaction profile, said method comprising: (a) creating a point-mutant epistatic miniarray profile (pE-MAP) and/or a chemical genetics miniarray profile (CG-MAP) associated with a first nucleic acid sequence and a second nucleic acid sequence by calculating an S-score associated with each of the first and second nucleic acid sequences; (b) correlating the genetic or chemical-genetic interaction profiles of the first nucleic acid sequence and the second nucleic acid sequence as a measure of similarity.

In some embodiments, the disclosure also relates to method of predicting effect of histone mutation on post-translational modifications of a histone protein, said method comprising: (a) creating a point-mutant epistatic miniarray profile (pE-MAP) or a chemical genetics miniarray profile (CG-MAP) associated with the histone protein; and (b) correlating the pE-MAP and/or the CG-MAP and the S-score, to a protein structural model of the histone protein; (c) identifying the effect to post-translational modifications by comparing the change in protein-protein interaction in a nucleic acid sequence comprising a mutation as relative to the protein-protein interaction of the same unmutated nucleic acid sequence.

In some embodiments, the method of predicting effect further comprises fitting a model of protein-protein interaction generated by the amount of a nucleic acid or amino acid in a system comprising a therapeutic agent.

In any of the above methods, method the correlating steps may comprise the substeps of: (i) calculating a plurality of MICs; (ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence; (iii) performing a noise model for calculation of a standard deviation; and (iv) formulating spatial restraints as Bayesian data likelihoods.

In any of the above steps, the upper distance bound is calculated by: (a) binning the plurality of MICs into from about 2 to about 1,000 intervals; (b) selecting a maximum distance spanned by any pair of amino acid residues in each bin; and (c) fitting a logarithmic decay function (du) to the upper distance bound:

$${d}_{U} ( MIC ) = \{ \begin{matrix}\frac{log ( MIC ) - n}{k} & if MIC \leq \dot{O} \\ i & if MIC > \dot{O}\end{matrix} \qquad ( 1 )$$

wherein n is a negative number from about 0 and to about −1; wherein k is a negative number between 0 and −1; wherein i is an interval number from about 5 to about 1,000, or from about 10 to about 40; and wherein O is from about 0.4 to about 0.8.

In some embodiments, O is about 0.4, about 0.5, about 0.6, about 0.7 or about 0.8.

In some embodiments, I is an interval number from about 10 to about 100, from about 20 to about 40, or from about 30 to about 40. In some embodiments, an interval number of about 10, about 20, about 30, about 40 or about 50.

In some embodiments, in any of the disclosed methods, the method further comprises substeps of further comprising calculating the likelihood of the entire pE-MAP or CG-MAP dataset, a product over the individual observations between residue pairs i, j:

p(D|M,I)=Π_{i,j}N(d_{i,j}|f_{i,j}(X),σ_{i,j})

wherein fi,j(X) is a forward model that predicts the data point di,j in D that would have been observed for structure X in an experiment without noise, and is defined as:

$${f}_{i , j} ( X ) = MIC ( {d}_{i , j} ) = \{ \begin{matrix}exp ( k \cdot {d}_{i , j} + n ) & if {d}_{i , j} \leq {d}_{0} \\ & if {d}_{i , j} > {d}_{0}\end{matrix} \qquad ( 2 )$$

wherein d0=du (from about 0.4 to about 0.8); and wherein N(di,j|fi,j(X),σi,j) is a noise model that quantifies the deviation between the predicted and observed data points and is a lognormal distribution with a flat plateau for MIC values below the upper bound on the observed MIC values:

$$P ( {MIC}_{i , j}^{obs} \vert MICi , j , X , {σ}_{i , j} ) = \{ \begin{matrix}\frac{1}{N} & if {MIC}_{i , j}^{obs} \geq {MIC}_{i , j} \\ \frac{1}{M} \frac{1}{\sqrt{2 {πσ}_{i , j}^{2}} {MIC}_{i , j}^{obs}} exp ( - \frac{1}{2 {σ}_{i , j}^{2}} {log}^{2} ( \frac{{MIC}_{i , j}^{obs}}{{MIC}_{i , j}} ) ) & if {MIC}_{i , j}^{obs} < {MIC}_{i , j}\end{matrix} \qquad ( 3 )$$

wherein σi,j are the noise parameters that can optionally be determined as part of the model, and N and M are normalization factors necessary to make the likelihood continuous.

In some embodiments, any of the methods disclosed can include a step of mapping, wherein the mapping of one or a plurality of protein-protein interactions has a resolution from about 1 to about 20 angstroms. In some embodiments, any of the methods disclosed can include a step of mapping, wherein the mapping of one or a plurality of protein-protein interactions has a resolution from about 5 to about 15 angstroms. In some embodiments, any of the methods disclosed can include a step of mapping, wherein the mapping of one or a plurality of protein-protein interactions has a resolution from about 10 to about 20 angstroms.

In some embodiments, any of the methods disclosed can include a step of mapping, wherein the mapping of one or a plurality of protein-protein interactions has a resolution of about 12, about 13, about 14, about 15, about 16, about 17, about 18, about 19 or about 20 angstroms.

Systems

The above-described methods can be implemented in any of numerous ways. For example, the embodiments may be implemented using a computer program product (i.e. software), hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

A computer employed to implement at least a portion of the functionality described herein may include a memory, coupled to one or more processing units (also referred to herein simply as “processors”), one or more communication interfaces, one or more display units, and one or more user input devices. The memory may include any computer-readable media, and may store computer instructions (also referred to herein as “processor-executable instructions”) for implementing the various functionalities described herein. The processing unit(s) may be used to execute the instructions. The communication interface(s) may be coupled to a wired or wireless network, bus, or other communication means and may therefore allow the computer to transmit communications to and/or receive communications from other devices. The display unit(s) may be provided, for example, to allow a user to view various information in connection with execution of the instructions. The user input device(s) may be provided, for example, to allow the user to make manual adjustments, make selections, enter data or various other information, and/or interact in any of a variety of manners with the processor during execution of the instructions.

The various methods or processes outlined herein may be coded as software that is executable on one or more processors that employ any one of a variety of operating systems or platforms. The disclosure also relates to a as a computer readable storage medium comprising executable instructions to perform any Additionally, such software may be written using any of a number of suitable programming languages and/or programming or scripting tools, and also may be compiled as executable machine language code or intermediate code that is executed on a framework or virtual machine.

In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention disclosed herein. The computer readable medium or media can be transportable, such that the program or programs stored thereon can be loaded onto one or more different computers or other processors to implement various aspects of the present invention as discussed above. In some embodiments, the system comprises cloud-based software that executes one or all of the steps of each disclosed method instruction.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the disclosure relates to various embodiments in which one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

In some embodiments, the disclosure relates to a system that comprises at least one processor, a program storage, such as memory, for storing program code executable on the processor, and one or more input/output devices and/or interfaces, such as data communication and/or peripheral devices and/or interfaces. In some embodiments, the user device and computer system or systems are communicably connected by a data communication network, such as a Local Area Network (LAN), the Internet, or the like, which may also be connected to a number of other client and/or server computer systems. The user device and client and/or server computer systems may further include appropriate operating system software.

In some embodiments, components and/or units of the devices described herein may be able to interact through one or more communication channels or mediums or links, for example, a shared access medium, a global communication network, the Internet, the World Wide Web, a wired network, a wireless network, a combination of one or more wired networks and/or one or more wireless networks, one or more communication networks, an a-synchronic or asynchronous wireless network, a synchronic wireless network, a managed wireless network, a non-managed wireless network, a burstable wireless network, a non-burstable wireless network, a scheduled wireless network, a non-scheduled wireless network, or the like.

Discussions herein utilizing terms such as, for example, “processing,” “computing,” “calculating,” “determining,” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes.

Some embodiments may take the form of an entirely hardware embodiment, an entirely software embodiment, or an embodiment including both hardware and software elements. Some embodiments may be implemented in software, which includes but is not limited to firmware, resident software, microcode, or the like.

Furthermore, some embodiments may take the form of a computer program product accessible from a computer-usable or computer-readable medium providing program code for use by or in connection with a computer or any instruction execution system. For example, a computer-usable or computer-readable medium may be or may include any apparatus that can contain, store, communicate, propagate, or transport the program for use by or in connection with the instruction execution system, apparatus, or device.

In some embodiments, the medium may be or may include an electronic, magnetic, optical, electromagnetic, InfraRed (IR), or semiconductor system (or apparatus or device) or a propagation medium. Some demonstrative examples of a computer-readable medium may include a semiconductor or solid state memory, magnetic tape, a removable computer diskette, a Random Access Memory (RAM), a Read-Only Memory (ROM), a rigid magnetic disk, an optical disk, or the like. Some demonstrative examples of optical disks include Compact Disk-Read-Only Memory (CD-ROM), Compact Disk-Read/Write (CD-R/W), DVD, or the like.

In some embodiments, a data processing system suitable for storing and/or executing program code may include at least one processor coupled directly or indirectly to memory elements, for example, through a system bus. The memory elements may include, for example, local memory employed during actual execution of the program code, bulk storage, and cache memories which may provide temporary storage of at least some program code in order to reduce the number of times code must be retrieved from bulk storage during execution.

In some embodiments, input/output or I/O devices (including but not limited to keyboards, displays, pointing devices, etc.) may be coupled to the system either directly or through intervening I/O controllers. In some embodiments, network adapters may be coupled to the system to enable the data processing system to become coupled to other data processing systems or remote printers or storage devices, for example, through intervening private or public networks. In some embodiments, modems, cable modems and Ethernet cards are demonstrative examples of types of network adapters. Other suitable components may be used.

Some embodiments may be implemented by software, by hardware, or by any combination of software and/or hardware as may be suitable for specific applications or in accordance with specific design requirements. Some embodiments may include units and/or sub-units, which may be separate of each other or combined together, in whole or in part, and may be implemented using specific, multi-purpose or general processors or controllers. Some embodiments may include buffers, registers, stacks, storage units and/or memory units, for temporary or long-term storage of data or in order to facilitate the operation of particular implementations.

Some embodiments may be implemented, for example, using a machine-readable medium or article which may store an instruction or a set of instructions that, if executed by a machine, cause the machine to perform a method steps and/or operations described herein. Such machine may include, for example, any suitable processing platform, computing platform, computing device, processing device, electronic device, electronic system, computing system, processing system, computer, processor, or the like, and may be implemented using any suitable combination of hardware and/or software. The machine-readable medium or article may include, for example, any suitable type of memory unit, memory device, memory article, memory medium, storage device, storage article, storage medium and/or storage unit; for example, memory, removable or non-removable media, erasable or non-erasable media, writeable or re-writeable media, digital or analog media, hard disk drive, floppy disk, Compact Disk Read Only Memory (CD-ROM), Compact Disk Recordable (CD-R), Compact Disk Re-Writeable (CD-RW), optical disk, magnetic media, various types of Digital Versatile Disks (DVDs), a tape, a cassette, or the like. The instructions may include any suitable type of code, for example, source code, compiled code, interpreted code, executable code, static code, dynamic code, or the like, and may be implemented using any suitable high-level, low-level, object-oriented, visual, compiled and/or interpreted programming language, e.g., C, C++, Java™, BASIC, Pascal, Fortran, Cobol, assembly language, machine code, or the like.

Many of the functional units described in this specification have been labeled as circuits, in order to more particularly emphasize their implementation independence. For example, a circuit may be implemented as a hardware circuit comprising custom very-large-scale integration (VLSI) circuits or gate arrays, off-the-shelf semiconductors such as logic chips, transistors, or other discrete components. A circuit may also be implemented in programmable hardware devices such as field programmable gate arrays, programmable array logic, programmable logic devices or the like.

In some embodiment, the circuits may also be implemented in machine-readable medium for execution by various types of processors. An identified circuit of executable code may, for instance, comprise one or more physical or logical blocks of computer instructions, which may, for instance, be organized as an object, procedure, or function. Nevertheless, the executables of an identified circuit need not be physically located together, but may comprise disparate instructions stored in different locations which, when joined logically together, comprise the circuit and achieve the stated purpose for the circuit. Indeed, a circuit of computer readable program code may be a single instruction, or many instructions, and may even be distributed over several different code segments, among different programs, and across several memory devices. Similarly, operational data may be identified and illustrated herein within circuits, and may be embodied in any suitable form and organized within any suitable type of data structure. The operational data may be collected as a single data set, or may be distributed over different locations including over different storage devices, and may exist, at least partially, merely as electronic signals on a system or network.

The computer readable medium (also referred to herein as machine-readable media or machine-readable content) may be a tangible computer readable storage medium storing the computer readable program code. The computer readable storage medium may be, for example, but not limited to, an electronic, magnetic, optical, electromagnetic, infrared, holographic, micromechanical, or semiconductor system, apparatus, or device, or any suitable combination of the foregoing. As alluded to above, examples of the computer readable storage medium may include but are not limited to a portable computer diskette, a hard disk, a random access memory (RAM), a read-only memory (ROM), an erasable programmable read-only memory (EPROM or Flash memory), a portable compact disc read-only memory (CD-ROM), a digital versatile disc (DVD), an optical storage device, a magnetic storage device, a holographic storage medium, a micromechanical storage device, or any suitable combination of the foregoing. In the context of this document, a computer readable storage medium may be any tangible medium that can contain, and/or store computer readable program code for use by and/or in connection with an instruction execution system, apparatus, or device.

The computer readable medium may also be a computer readable signal medium. A computer readable signal medium may include a propagated data signal with computer readable program code embodied therein, for example, in baseband or as part of a carrier wave. Such a propagated signal may take any of a variety of forms, including, but not limited to, electrical, electro-magnetic, magnetic, optical, or any suitable combination thereof. A computer readable signal medium may be any computer readable medium that is not a computer readable storage medium and that can communicate, propagate, or transport computer readable program code for use by or in connection with an instruction execution system, apparatus, or device. As also alluded to above, computer readable program code embodied on a computer readable signal medium may be transmitted using any appropriate medium, including but not limited to wireless, wireline, optical fiber cable, Radio Frequency (RF), or the like, or any suitable combination of the foregoing. In one embodiment, the computer readable medium may comprise a combination of one or more computer readable storage mediums and one or more computer readable signal mediums. For example, computer readable program code may be both propagated as an electro-magnetic signal through a fiber optic cable for execution by a processor and stored on RAM storage device for execution by the processor.

Computer readable program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The computer readable program code may execute entirely on a user's computer, partly on the user's computer, as a stand-alone computer-readable package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The program code may also be stored in a computer readable medium that can direct a computer, other programmable data processing apparatus, or other devices to function in a particular manner, such that the instructions stored in the computer readable medium produce an article of manufacture including instructions which implement the function/act specified in the schematic flowchart diagrams and/or schematic block diagrams block or blocks.

Functions, operations, components and/or features described herein with reference to one or more embodiments, may be combined with, or may be utilized in combination with, one or more other functions, operations, components and/or features described herein with reference to one or more other embodiments, or vice versa.

Although the disclosure refers to exemplary embodiments, it is not limited thereto. Those skilled in the art will appreciate that numerous changes and modifications may be made to the preferred embodiments of the disclosure and that such changes and modifications may be made without departing from the true spirit of the disclosure. It is therefore intended that the appended claims be construed to cover all such equivalent variations as fall within the true spirit and scope of the disclosure.

All referenced journal articles, patents, and other publications are incorporated by reference herein in their entireties.

### EXEMPLIFICATION

Representative examples of the disclosed methods and systems are illustrated in the following non-limiting methods and examples.

### Example 1. Genetic Interaction Mapping Informs Integrative Determination of Biomolecular Assembly Structures

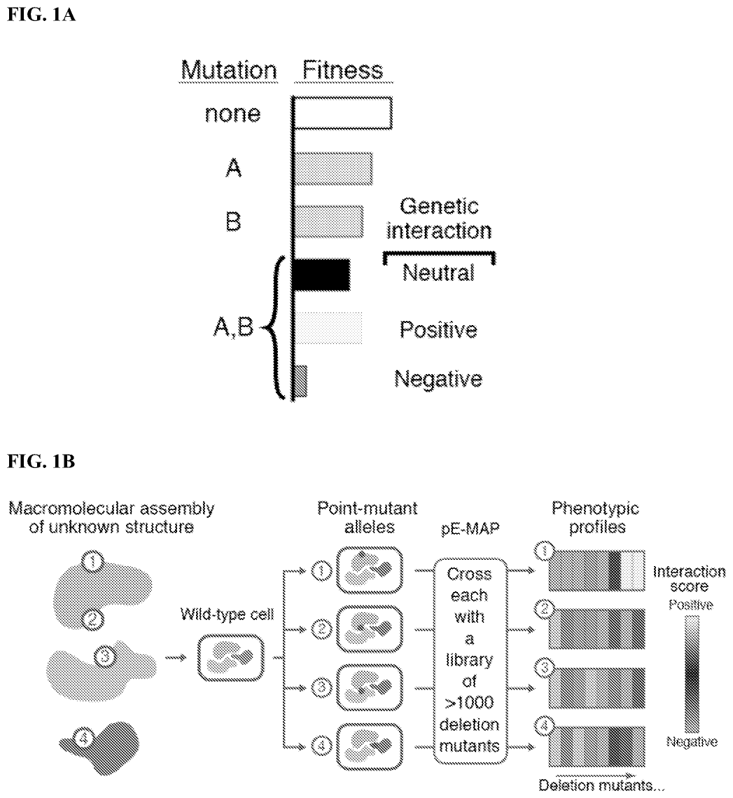

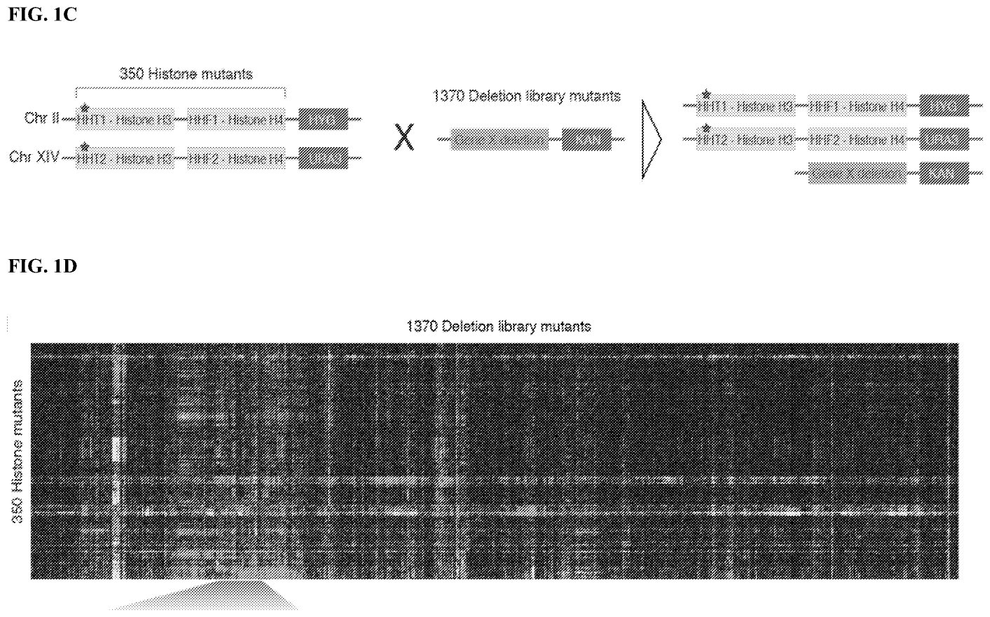

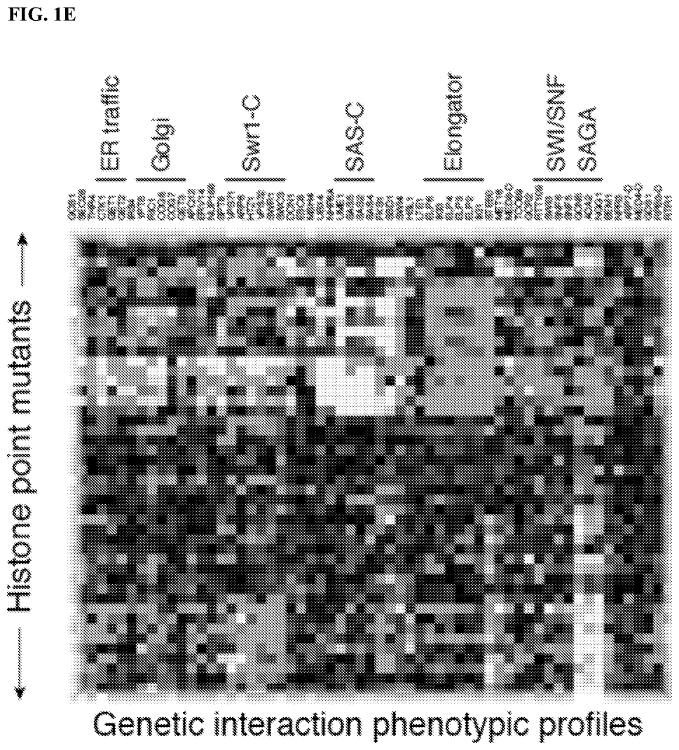

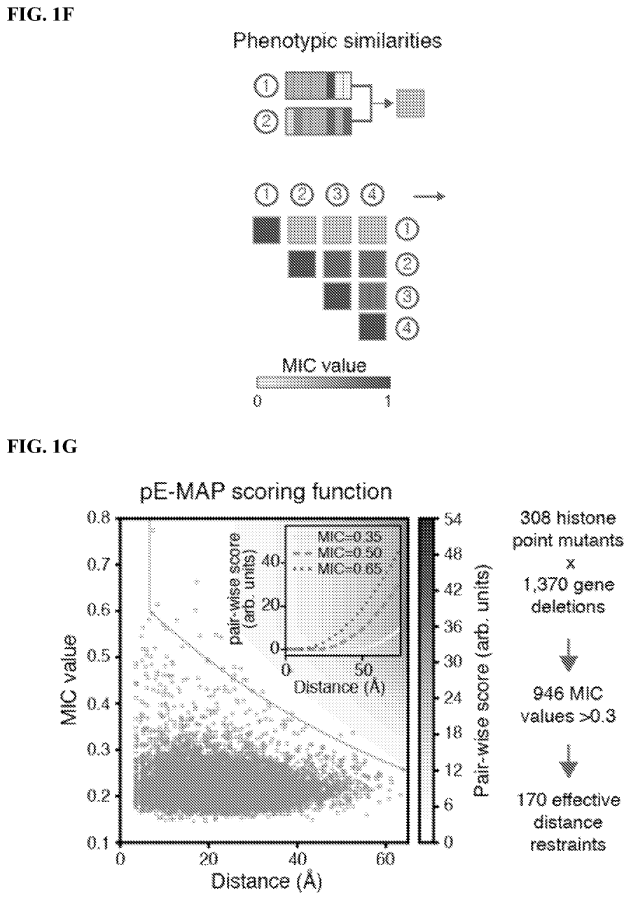

Determining structures of macromolecular assemblies is crucial for understanding cellular functions. Here, we describe how integrative structure modeling can benefit critically from spatial restraints derived from in vivo quantitative measurements of genetic interactions. A genetic interaction between two mutations occurs when the effect of one mutation is altered by the presence of the second mutation (FIG. 1A) (13). Positive genetic interactions (epistasis/suppression) arise when the double mutant is healthier than expected, whereas negative interactions (synthetic sickness) arise in relationships where the double mutant is sicker than expected. Single genetic interactions can be difficult to interpret in isolation. A phenotypic profile, defined as a set of genetic interactions between a given mutation (e.g. a point mutation) and a library of secondary mutations (e.g. gene deletions), can be more informative (FIG. 1B). A point-mutant epistatic mini-array profile (pE-MAP) is comprised of such phenotypic profiles for all mutations in the analysis. We have previously found a statistical association of the distance between two mutated residues in the WT structure and the similarity between their phenotypic profiles (i.e. phenotypic similarity) (14, 15). This observation is in agreement with the expectation that mutations within the same functional region (e.g. active, allosteric, and binding sites) are likely to share more similar phenotypes than those that are distant in space (16-18). Here, we explore how to use these associations for determining in vivo structures of macromolecular assemblies. To enable this analysis, we generated the largest pE-MAP to date, by designing a comprehensive set of 350 mutations in histones H3 and H4, and crossing these against 1,370 gene deletions. We describe this pE-MAP and illustrate integrative structure determination by its application to three complexes of known structure; (i) the yeast histones H3-H4; (ii) subunits Rpb1 and Rpb2 of yeast RNA polymerase II (RNAPII), using a pE-MAP dataset of 53 point mutants crossed against a library of 1,200 gene deletions (14), and (iii) subunits RpoB and RpoC of bacterial RNA polymerase (RNAP), using a chemical genetics map (CG-MAP), where 44 point mutants were subjected to 83 different environmental stresses (e.g. treatments with chemicals and temperature shocks) (Shiver et al., in preparation). Two recent studies have shown that deep mutational scans can be used for determining the fold of small protein domains at a high resolution (19, 20). While deep mutational studies subject cells to a small number of selective conditions that are usually known to target the system of interest, pE-MAPs and CG-MAPs are generated using large libraries of perturbations, thus expanding the scope of the genetic interactions identified.

Materials and Methods

Strain Construction

The histone H3/H4 mutant strain library was constructed as described (22, 23). Briefly, 382 histone H3 and H4 mutants (tail deletions, complete alanine-scan, and context specific point mutations) were generated in the MSY196 background (MATα his3Δleu2Δ ura3Δ can1::STE2pr-spHIS5 lyp1::STE3pr-LEU2) (Table S1). First, the base strains were created by replacing the HHT2-HHF2 locus with a URA3-containing cassette carrying a mutated HHT2-HHF2 locus with their endogenous promoters. After sequence validation of each base strain, the HHT1-HHF1 locus was replaced with a HYG^{R}-containing cassette including a mutated version of the HHT1-HHF1 locus, resulting in pE-MAP-amenable strains (Mata his3Δ leu2Δ ura3Δ hht1-hhf1::HYG^{R} hht2-hhf2:: URA3 can1::STE2pr-spHIS5 lyp1::STE3pr-LEU2).

Library Validation

Libraries were validated in three steps: (1) each mutant was constructed, transformed into bacteria, and sequenced; 100% sequence identity was required to pass quality control; (2) after plasmid isolation from yeast, 5-10 randomly selected constructs from each 96-well plate were sequenced to ensure the identity of each mutant in the well and no cross-contamination during plasmid preparation; 100% of these were correct; (3) after we obtained the yeast library, we PCR-amplified the individual integrated constructs followed by sequencing to confirm the identity of mutations. This last step was performed for all the lethal mutants.

pE-MAP Analysis

Each of the histone H3/H4 mutant strains was crossed with 1370 MATa KAN^{R} marked deletion/DAmP strains by pinning on solid media essentially as described (75). Sporulation was induced and MATa haploid spores were selected by replica plating onto media containing canavanine (selecting can1Δ haploids) and S-AEC (selecting lyp1Δ haploids) and lacking histidine (selecting MATa spores). Triple mutant haploids were isolated on media containing hygromycin (selecting hht1-hhf1 mutant cassette) and G418 (selecting KAN^{R} marked deletion/DAmP), and lacking uracil (selecting hht2-hhf2 mutant cassette). Finally, triple mutant colony sizes were extracted using imaging software, and genetic interaction scores computed using a statistical scoring scheme described in (28). Detailed E-MAP experimental procedures are described in (26, 76, 77).

The yeast RNAPII pE-MAP dataset is described in (14) and the description of the bacterial RNA polymerase dataset is in preparation (Anthony L. Shiver, Hendrik Osadnik, Jason M. Peters, Rachel A. Mooney, Robert Landick, Kerwyn Casey Huang, Carol A. Gross).

Design of the pE-MAP Spatial Restraints

The distance restraint based on pE-MAP data was designed using the 308 single point mutants from the histone pE-MAP and the structure from the PDB (1ID3), as follows: 1) post-processing of the genetic interaction phenotypic profiles, 2) devising a phenotypic similarity metric between the phenotypic profiles, and 3) designing spatial restraints for integrative structure modeling using the phenotypic similarity values and the known histone X-ray structure. Next, we describe each of these three steps in turn.

(1) Post-Processing of the Genetic Interaction Phenotypic Profiles

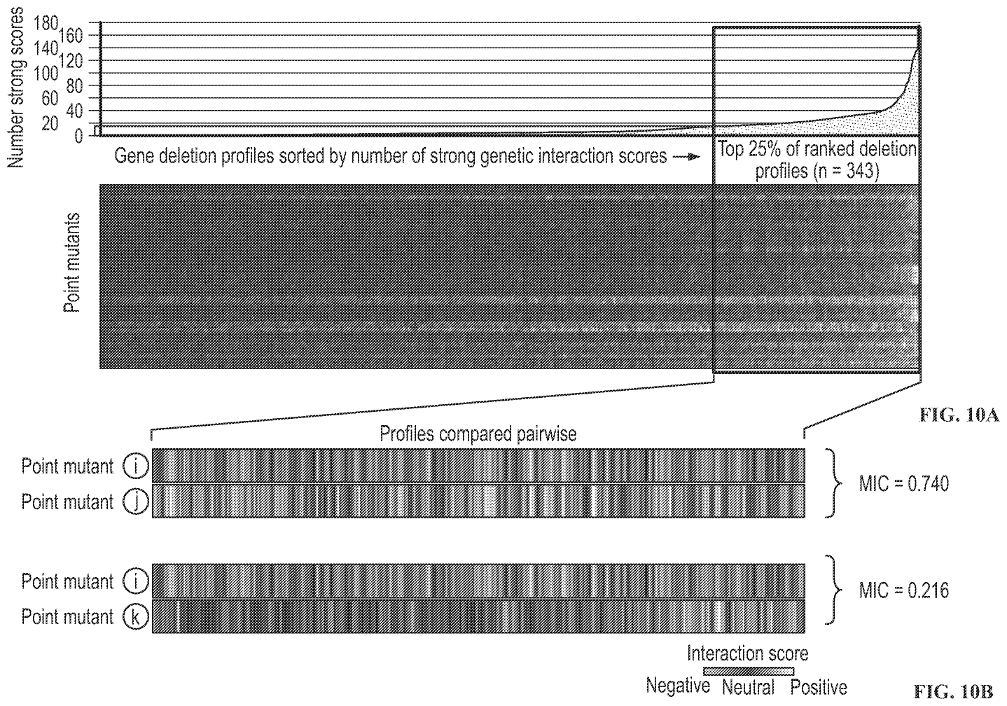

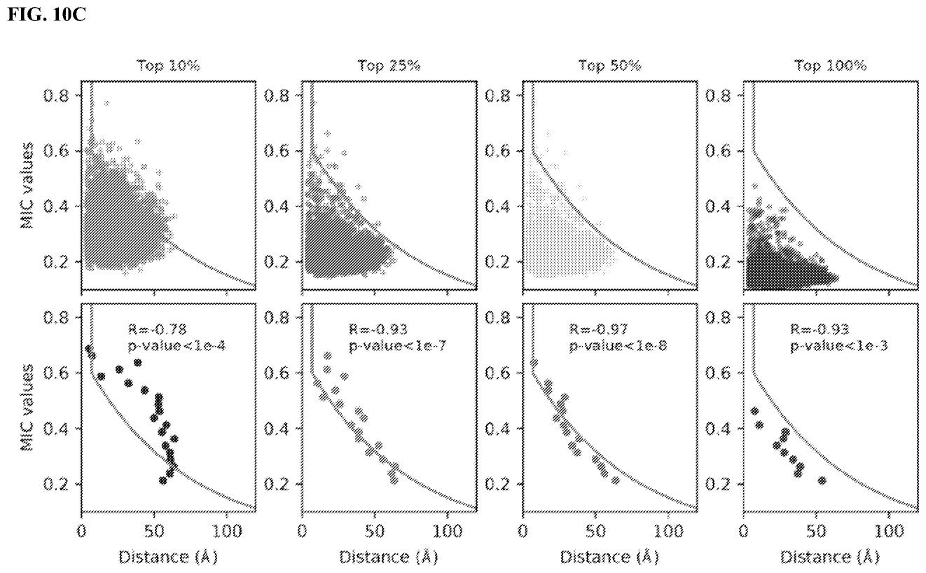

All missing values in the pE-MAP were imputed as the mean of the genetic interaction scores between the corresponding deletion mutant and all histone mutants. To increase the signal-to-noise ratio of the pE-MAP, gene deletions that mostly exhibited weak genetic interactions with the histone mutants were filtered out. To this end, the gene deletion profiles were ranked in descending order based on the counts of their genetic interaction scores that fell in either the top 2.5% of positive scores or the bottom 5% of negative scores, from the complete pE-MAP (cutoffs calculated after imputation). The more stringent cutoff for positive scores was chosen to reflect the smaller dynamic range for positive genetic interactions compared to negative genetic interactions. Gene deletions with the same count were then ranked in descending order by the mean of the absolute values of their highest and lowest score (FIG. 10A). The top fraction of the deletions, determined in step 3 below, were retained for computing the histone point mutant phenotypic profile similarities (below, FIG. 10).

(2) Devising a Phenotypic Similarity Metric Between the Phenotypic Profiles

We computed the similarity between all pairs of histone phenotypic profiles using the maximal information coefficient (MIC, FIG. 10B), with the MIC parameters alpha and c set to 0.6 and 15, respectively, as suggested (34, 35). Many positions in the histones were mutated to several different residue types, giving rise to several phenotypic profiles for each of these positions. As a result, more than one MIC value would often be computed for a single residue pair. In such cases, only the highest MIC value was retained.

(3) Designing Spatial Restraints for Integrative Structure Modeling

Using the histone X-ray structure ((36); PDB: 1ID3), we measured the distance between all pairs of residues for which we computed a MIC phenotypic similarity score. The percentage of the top scoring phenotypic profiles (ranked by the genetic interaction scores) retained for further analysis was determined as follows. We compared the statistical association of the distances between two mutated residues with their phenotypic similarity by selecting the top 10%, 25%, 50%, and 100% of the ranked deletions (FIG. 10C). Although MIC values between phenotypic profiles do not linearly correlate with the distances spanned by the mutated residues in the WT structure (Pearson correlation coefficient of −0.07 when using the top 25% or top 50% of deletions), the MIC values provide an upper distance bound between the residues. The upper distance bound was obtained by binning the MIC values into 20 intervals and selecting the maximum distance spanned by any pair of residues in each bin, followed by fitting a logarithmic decay function (dᵤ) to the upper distance bounds:

$${d}_{U} ( MIC ) = \{ \begin{matrix}\frac{log ( MIC ) - n}{k} & if MIC \leq 0.6 \\ 20 & if MIC > 0.6\end{matrix} \qquad ( 1 )$$

Where n and k are −0.0147 and −0.41, respectively (FIG. 10C). We find that selecting the top 25% or 50% of the deletions had a comparable association between the upper distance bounds and the computed MIC values. In this work, we retained the top 25% of the ranked phenotypic profiles for computing the phenotypic profile similarities.

To effectively handle the uncertain relationship between the data and model, we use Bayesian inference for scoring alternative models by formulating spatial restraints as Bayesian data likelihoods (78). Formally, the posterior probability of model M given data D and prior information I is

p(M|D,I)∝p(D|M,I)·p(M|I)

The model, M, consists of a structure X and unknown parameters Y, such as noise in the data. The prior p(M|I) is the probability density of model M given I. The prior can in general reflects information such as statistical potentials and a molecular mechanics force field; here, we only used excluded volume and sequence connectivity. The likelihood function p(D|M, I) is the probability density of observing data D given M and I. The pE-MAP data was used to compute phenotypic similarities (i.e. MIC values) that inform distances between mutated residues pairs i, j. The likelihood of the entire pE-MAP dataset is the product over the individual observations between residue pairs i, j:

p(D|M,I)=Π_{i,j}N(d_{i,j}|f_{i,j}(X),σ_{i,j})

where f_{i,j}(X) is a forward model that predicts the data point d_{i,j} in D that would have been observed for structure X in an experiment without noise; N(d_{i,j}|f_{i,j}(X),σ_{i,j}) is a noise model that quantifies the deviation between the predicted and observed data points.

We defined the forward model by inverting the relation between the upper distance bound and observed MIC values (dᵤ (MIC), Eq. 1):

$${f}_{i , j} ( X ) = MIC ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$

where d₀=dᵤ (0.6). Our choice of a noise model is a lognormal distribution with a flat plateau for MIC values below the upper bound on the observed MIC values:

$$P ( {MIC}_{i , j}^{obs} \vert MICi , j , X , {σ}_{i , j} ) = \{ \begin{matrix}\frac{1}{N} & if {MIC}_{i , j}^{obs} \geq {MIC}_{i , j} \\ \frac{1}{M} \frac{1}{\sqrt{2 {πσ}_{i , j}^{2}} {MIC}_{i , j}^{obs}} exp ( - \frac{1}{2 {σ}_{i , j}^{2}} {log}^{2} ( \frac{{MIC}_{i , j}^{obs}}{{MIC}_{i , j}} ) ) & if {MIC}_{i , j}^{obs} < {MIC}_{i , j}\end{matrix} \qquad ( 3 )$$

Here, σ_{i,j} are the noise parameters that can optionally be determined as part of the model, and N and M are normalization factors necessary to make the likelihood continuous. Lognormal noise models have previously been used to describe errors of inherently positive quantities (79). For computational efficiency, we used a single σ value for all residue pairs. An uninformative Jeffrey's prior is applied to σ to represent a lack of information on the bounds and distribution of this parameter (80).

Finally, a Bayesian term in the scoring function is defined as the negative logarithm of the posterior probability density: S(M)=−log p(M|D, I). In the Bayesian view, the output model is in fact best equated to the posterior model density that specifies a distribution of alternative single models M with varying probability density, not a single model, although single representative or average models can always be proposed based on the posterior model density.

Calculation of Similarity Metrics for Yeast RNAPII and Bacterial RNAP Datasets

Steps 1) and 2) from “Design of the pE-MAP Spatial Restraints” were repeated for the yeast RNAPII and bacterial RNAP datasets to generate the similarity metrics (MIC values) for these two systems, with the following modifications.

For yeast RNAPII, prior to imputing missing values in the pE-MAP, any deletion mutants that exhibit missing values with more than 15% of the point mutants were filtered out. This step is part of our pipeline, but has no effect on the histone pE-MAP, because this pE-MAP does not contain any deletion mutant with more than 15% values missing. The number of ranked deletion mutants retained at the end of pE-MAP post-processing was chosen to be 25% of the number of deletions in the original unfiltered pE-MAP (in accordance with the histone pE-MAP processing).

For bacterial RNAP, due to the very small number of perturbations in this dataset, the top 50% (instead of 25%) of the ranked perturbations were retained for computing point mutant phenotypic profile similarities. In addition, due to differences in the experimental design for determining the yeast pE-MAP and the bacterial RNAP CG-MAP, the bacterial RNAP MIC distribution had a ˜2-fold higher median and greater spread than the other datasets. Correspondingly, the bacterial RNAP MIC distribution was normalized using linear scaling, decreasing its median to match that of the histone MICs. Importantly, this step was based solely on the MIC distributions, without reliance on any structural information.

Integrative Structure Determination

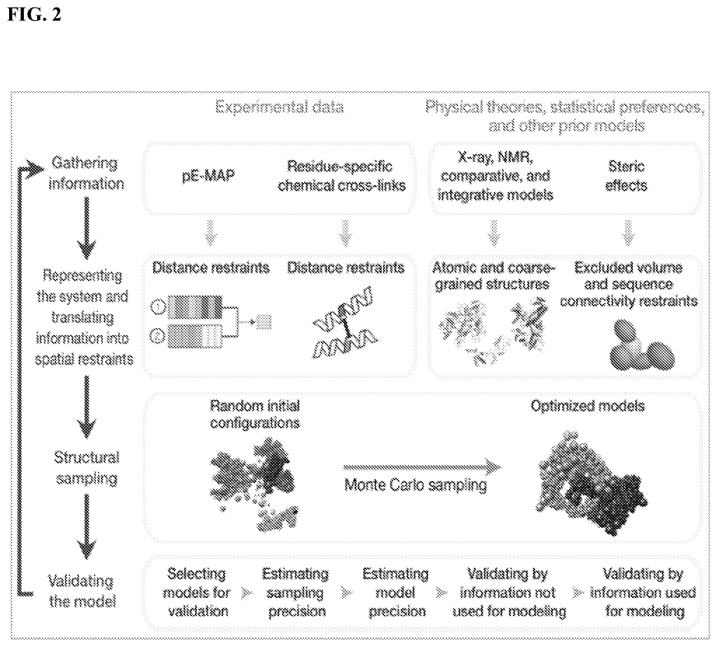

Integrative structure determination for each system proceeded through the standard four stages (4, 5, 8, 58, 81, 82) (FIG. 2, Tables 2, 4, 5): (1) gathering data, (2) representing subunits and translating data into spatial restraints, (3) configurational sampling to produce an ensemble of structures that satisfies the restraints, and (4) analyzing and validating the ensemble structures and data. The integrative structure modeling protocol (i.e. stages 2, 3, and 4) was scripted using the Python Modeling Interface (PMI) package, a library for modeling macromolecular complexes based on our open-source Integrative Modeling Platform (IMP) package (5), version 2.8 (https://integrativemodeling.org). Files containing the input data, scripts, and output results are available at https://github.com/salilab/pemap and the nascent integrative methods benchmarking section of the worldwide Protein Data Bank (wwPDB) PDB-Dev repository for integrative structures and corresponding data (pdb-dev.wwpdb.org) (83).

(1) Gathering Data

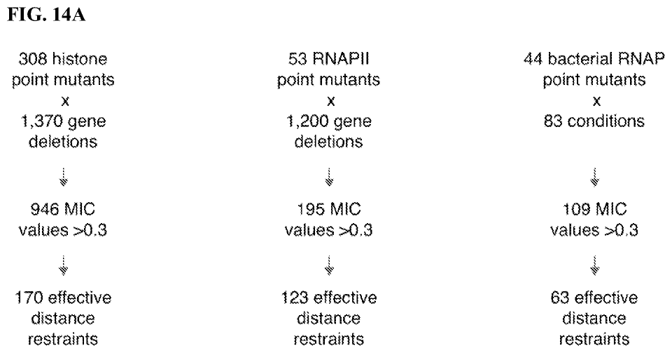

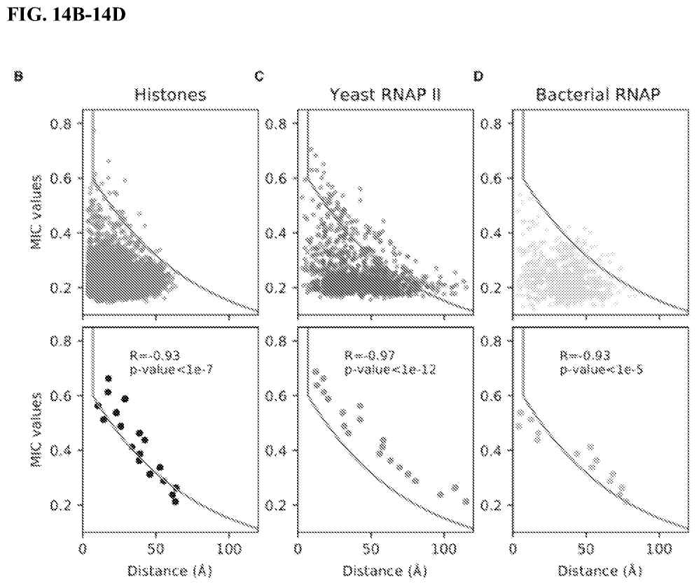

To mimic realistic integrative structure determination, we did not rely on the known atomic structures of the subunits in the actual modeled complex (correct docking of exact bound structures based on geometric complementarity is easy, (84)). Instead, we computed comparative models of histones H3 and H4 based on their alignments with structures of the 1TZY (85) (89% and 92% sequence identity, respectively), using MODELLER, version 9.21 (86). The Cα-atom RMSDs between the crystal structures and comparative models range between 2.8 and 5.5 Å, corresponding to medium and low accuracy comparative models. The second major input information source was a pE-MAP dataset of 308 point mutations in histones H3 and H4 crossed against an array of ˜1,370 gene deletion alleles, resulting in 946 MIC values above 0.3. Of these, 170 MIC values were converted into distance restraints between H3 and H4 residues (Table 2, FIG. 14).

Comparative models of subunits Rpb1 and Rpb2 of yeast RNAPII were computed based on template structures 6GMH (87) (54% sequence identity) and 4AYB (88) (43% sequence identity), respectively. The Cα RMSD between the crystal structures of subunit Rpb1 and Rpb2 (1I3Q, (89)) and their comparative models are 7.3 and 5.2 Å, respectively. A pE-MAP dataset of 53 single point mutants in subunits Rpb1 and Rpb2 of yeast RNA polymerase II (RNAPII) and a library of ˜1,200 gene-deletions resulted in 195 MIC values above 0.3. Of these, 123 MIC values were converted into distance restraints (Table 4, FIG. 14. In addition, we compared the RNAPII model based on the pE-MAP to a model based on 22 previously published chemical cross-links (XLs) (44).

The structures of subunits RpoB and RpoC of bacterial RNA polymerase (RNAP) were obtained from the X-ray structure of the entire complex (4YG2) (90). A CG-MAP of 44 single point mutants of the two subunits and a library of 83 conditions (e.g. treatments with chemicals and temperature shocks) resulted in 109 MIC values above 0.3. Of these, 63 MIC values were converted into distance restraints between the subunits (Table 5, FIG. 14). In addition, we compared the bacterial RNAPII model based on the CG-MAP to a model computed by IMP based on distance restraints derived from the interfacial contacts predicted using the RaptorX protein complex contact prediction server (48, 49).

(2) Representing Subunits and Translating Data into Spatial Restraints

To maximize computational efficiency while avoiding using too coarse a representation, we represented each complex in a multi-scale fashion. In particular, the subunits/domains of each complex were coarse-grained using beads of varying sizes representing either a rigid body or a flexible string, based on the available comparative models, as follows (Tables 2, 4, 5). The comparative models were coarse-grained into two representations at different resolutions. First, we identified loop regions of at least 8 residues using DSSP (91, 92) and represented them by flexible strings of beads of up to 10 residues each. Second, for the remaining residues each bead corresponded to an individual residue, centered at the position of its Cα atom. With this representation in hand, we next translated the input information into spatial restraints as follows.

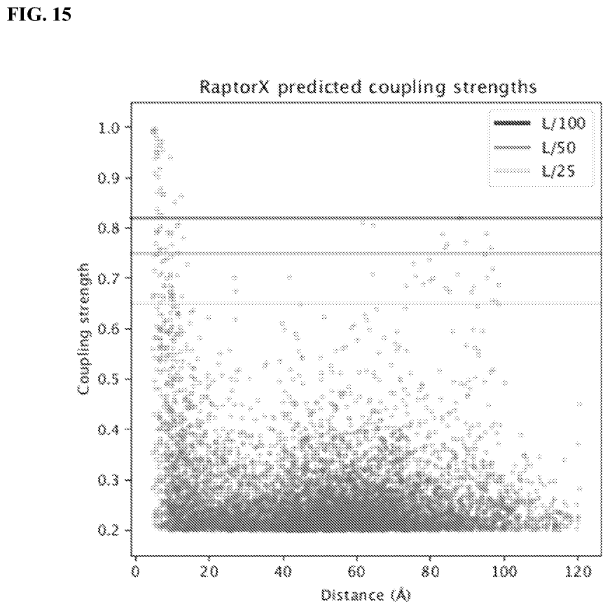

The defining and most important restraint for our method is extracted from the pE-MAP/CG-MAP. The collected pE-MAP and CG-MAP MIC values were used to construct the Bayesian term in the scoring function that restrained the distances spanned by the mutated residues as described above. The pE-MAP restraint was applied to the one residue-per-bead representation for the comparative models as well as to the flexible beads. To improve computational efficiency, we only considered point mutation pairs with MIC values larger than 0.3. This restraint was applied to all three complexes (Tables 2, 4, 5). In addition to the pE-MAP data, integrative modeling can benefit from many other types of input information. Here, we have supplemented the pE-MAP/CG-MAP data by additional simple terms accounting for excluded volume and sequence connectivity. First, the excluded volume restraints were applied to each bead in the one-residue (or the closest) bead representations, using the statistical relationship between the volume and the number of residues that it covered (4, 93). Second, we applied the sequence connectivity restraint, using a harmonic upper bound on the distance between consecutive beads in a subunit, with a threshold distance equal to four times the sum of the radii of the two connected beads. The bead radius was calculated from the excluded volume of the corresponding bead, assuming standard protein density (4, 93). Moreover, we evaluated the utility of pE-MAP/CG-MAP data by considering two additional types of restraints. First, the 22 previously determined BS3 RNAPII cross-links (44) were used to construct a Bayesian term that restrained the distances spanned by the cross-linked residues (30 Å) (94, 95). The cross-link restraints were applied to the one residue-per-bead representation for the comparative models as well as flexible beads, only for RNAPII (Table 4). Second, we applied the evolutionary coupling restraints to determine the structures of the RpoB and RpoC subunits of bacterial RNA polymerase. Coupling strengths between residue pairs were obtained using the RaptorX ComplexContact server (http://raptorx.uchicago.edu/ComplexContact/) (48, 49) with default parameters. The top L/50 coupling strengths (FIG. 15) with sequence separation of 3 or greater were converted into distance restraints using a harmonic upper bound on the distances between the residues. The threshold distance was set to 12 Å. This restraint was applied only to a subset of bacterial RNAP modeling instances (Table 5).

Configurational Sampling to Produce an Ensemble of Structures that Satisfy the Restraints

The initial positions and orientations of rigid bodies and flexible beads were randomized. The generation of structural models was performed using Replica Exchange Gibbs sampling, based on the Metropolis Monte Carlo (MC) algorithm (95, 96). Each MC step consisted of a series of random transformations (i.e. rotation and translation) of the positions of the flexible beads and rigid bodies. Details about the Monte Carlo runs for each system are in Tables 2, 4, 5.

Analyzing and Validating the Ensemble Structures and Data

Model validation follows four major steps (3, 97): (i) selection of the models for validation; (ii) estimation of sampling precision; (iii) estimation of model precision, and (iv) quantification of the degree to which a model satisfies the information used to compute it. These validations are based on the nascent wwPDB effort on archival, validation, and dissemination of integrative structures (83, 98). We now discuss each one of these validations in turn.

(1) Selection of Models for Validation

The first step is to objectively define the ensemble of models that will be further analyzed. For each trajectory, we automatically determined the MC step at which all data likelihoods and priors have equilibrated (run equilibration step); and all prior frames are discarded (99). Discarding the initial, non-equilibrated steps of each run is helpful because non-typical early configurations (e.g. a random configuration of beads, an extended configuration of beads, and beads far apart from each other) are removed from the statistical sample used for posterior model estimates.

With this ensemble of sampled structures and their corresponding scores in hand, we analyze the data likelihoods and priors. We used HDBSCAN clustering, a hierarchical density-based clustering algorithm, to identify all high-density regions in the likelihoods and priors (100). If a single cluster was identified, we consider all the models after discarding the initial steps; otherwise, we consider all models in the clusters that satisfy the input information, within the uncertainty of the data, for further analysis (below).

(2) Estimation of Sampling Precision

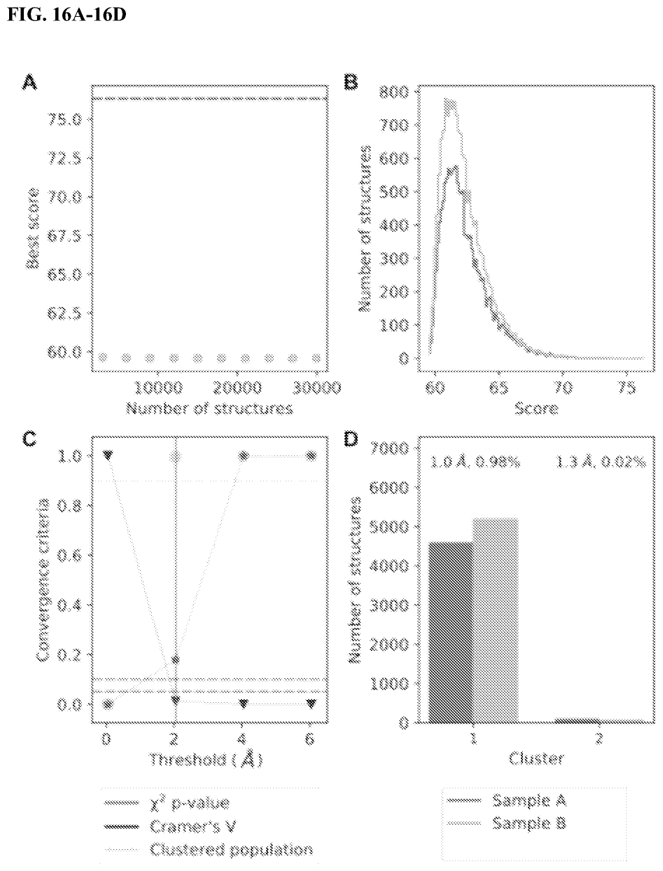

Next, we estimate the precision at which sampling sampled the selected structures (sampling precision) (97); the sampling precision must be at least as high as the precision of the structure ensemble consistent with the input data (model precision). As a proxy for testing the thoroughness of sampling, we performed four sampling convergence tests: 1) verify that the scores of refined structures do not continue to improve as more structures are computed, 2) confirm that the selected structures in independent sets of sampling runs (Sample A and Sample B) satisfy the data equally well, 3) cluster the structural models and determine the sampling precision at which the structural features can be interpreted (FIG. 16), and 4) compare the localization probability density maps for each protein obtained from independent sets of runs. Details about all the tests are described in (97). For each modeling instance, the results from the convergence tests are summarized in Tables 2, 4, 5.

(3) Estimation of Model Precision:

In the third step, model uncertainty (precision) is estimated. The most explicit description of model uncertainty is provided by the set of all models that are sufficiently consistent with the input information (i.e. the ensemble). Model precision can be quantified by the variability among the models in the ensemble; in the end, the ensemble can be described by one or more representative models and their uncertainties. For example, if the structures is the ensemble are clustered into a single cluster, the model precision is defined as the RMSD between models in the cluster. Importantly, the uncertainty may not be distributed evenly across the ensemble, such that some regions are determined at a higher precision than others.

(4) Quantification of the Degree to which a Model Satisfies the Data Used to Compute it

An accurate structure needs to satisfy the input information used to compute it; all structures at computed precision that are consistent with the data are provided in the ensemble. A pE-MAP derived restraint is satisfied by a cluster of structures if the corresponding Cα-Cα distance in any of the structures in the cluster is lower than the distance predicted by the MIC value (Eq. 1). A BS3 cross-link restraint is satisfied by a cluster of structures if the corresponding Cα-Cα distance in any of the structures in the cluster is less than 30 Å (101). The remainder of the restraints are harmonic, with a specified standard deviation. Therefore, a restraint is satisfied by a cluster of structures if the restrained distance in any structure in the cluster is violated by less than 3 standard deviations, specified for the restraint. Tables 2, 4 and 5 show that all models satisfy the input information within its uncertainty.

Benchmark

To benchmark the four-stage protocol described above, we computed the distribution of the accuracy for each structure in the ensemble. The accuracy is defined as the mean of Cα root-mean-square deviation (RMSD) between the X-ray structure and each of the structures in the ensemble. The PDB accession code and accuracies for each modeling instance are summarized in Tables 2, 4, and 5.

To assess the information content of the histone pE-MAP, we computed the models of the H3-H4 complex based on random subsets of the data. To this end, from the dataset of computed MIC values for pairs of mutated residues, we performed three independent random selections of 80%, 60%, 40%, and 20% of the data each. As expected, the more pE-MAP data used, the more accurate and precise are the models (Table 2).

Finally, as another test, we computed the model based on datasets with randomly shuffled MIC values for the same pE-MAP/CG-MAP residue pairs, for each of the complexes.

Estimation of the Number of Mutations Per Protein

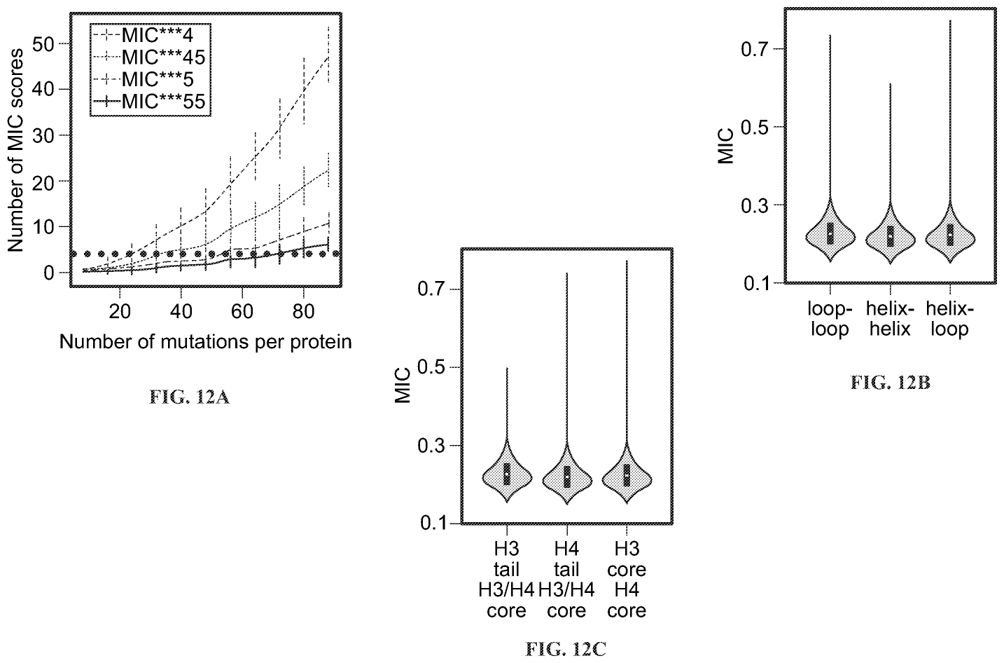

To estimate the suggested number of mutated positions per protein for integrative structure determination, we computed the number of mutations that would result in 4 or more MIC values above a 0.4, 0.45, 0.5, or 0.55 threshold. Based on our scoring function, MIC values above these thresholds will result in distance restraints with upper bounds in the 12-34 Å range. These distances are comparable to the distance upper bounds used for chemical cross-links (e.g. DSS, DSSO, EDC). A previous systematic study established that at least 4 chemical cross-links are needed to determine the binding mode of protein dimers if the subunit structures are known (e.g. from X-ray, NMR, or comparative models) (95). In general, adding more chemical cross-links does not further improve the accuracy, although it increases the precision of the resulting ensemble. By analogy, we estimate that, for systems in which the structures of the components are known, a good number of mutations per protein is 35-40 (FIG. 12A). This data can be used as a guideline to decide on the number of mutations to use for generating a pE-MAP or CG-MAP. Importantly, this estimate is an upper bound on the number of mutations, and in many cases, the number might be smaller for the following two reasons. First, this estimation was done assuming protein-wide mutations of residues, often to alanine. In practice, the number of necessary mutations can be reduced by specifically designing point mutations that target surface residues and/or residues known to be functionally important, and by choosing substitutions likely to give rise to functional perturbations. In general, we did not find a correlation between the secondary structure of the residue pairs and their associated MIC value (FIG. 12B). Second, this estimation only relies on the residue pairs with high MIC values. In contrast to chemical cross-links, the upper distances of pE-MAP derived restraints are obtained from the statistical association between the MIC values and distance between residues. Consequently, residue pairs with low MIC values still carry structural information, even if at low resolution. Consistent with these considerations, the RNAPII dataset contains only 34 and 9 mutations for Rpb1 and Rpb2, respectively. Similarly, the bacterial RNAP dataset contains 23 and 15 mutations for Rpob and Rpoc, respectively.

Docking

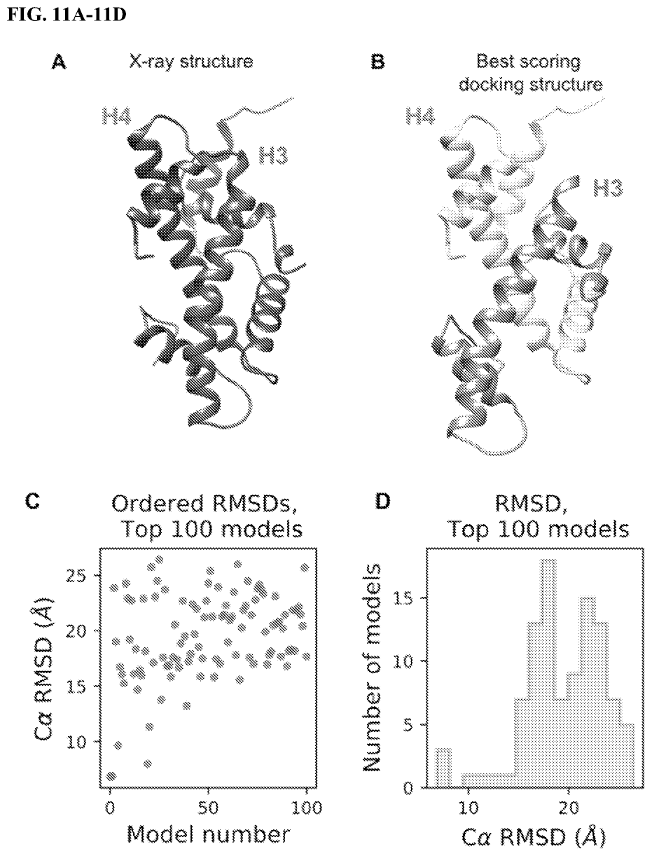

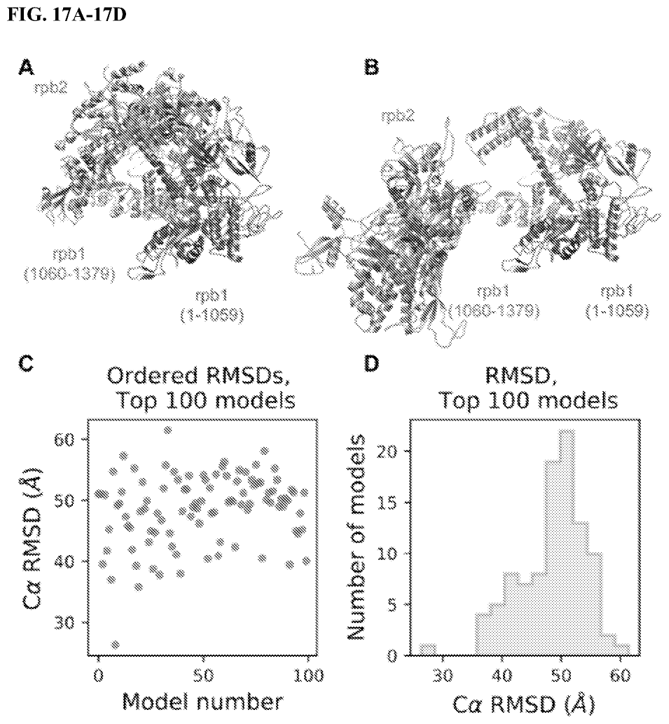

To assess the relative value of pE-MAP/CG-MAP restraints for structure determination, we computed the structures of the H3-H4 and RNAPII complexes by molecular docking. Specifically, we followed an integrative docking protocol (102) using the rigid-body docking program PatchDock (37). In each case, we used the same comparative models and rigid body definitions used for integrative modeling (FIGS. 11 and 17) and default parameter values.

Visualizations

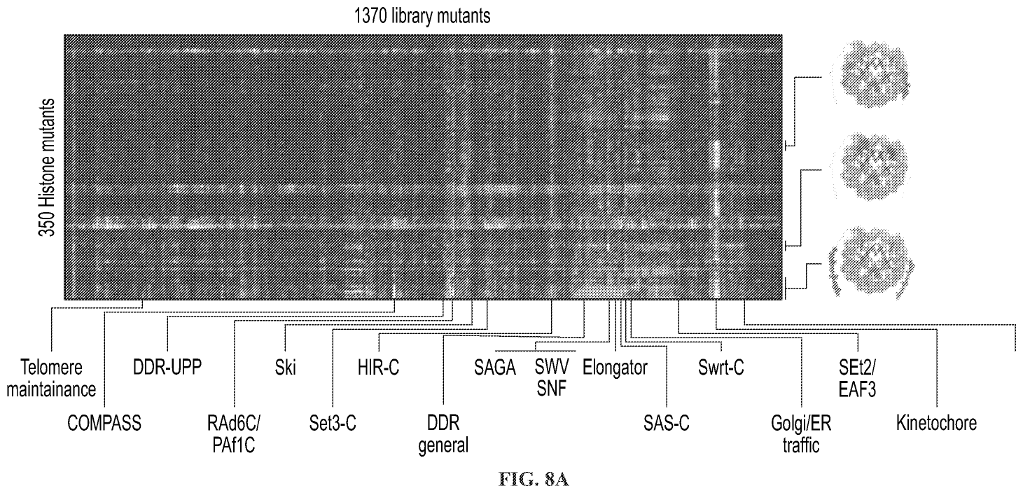

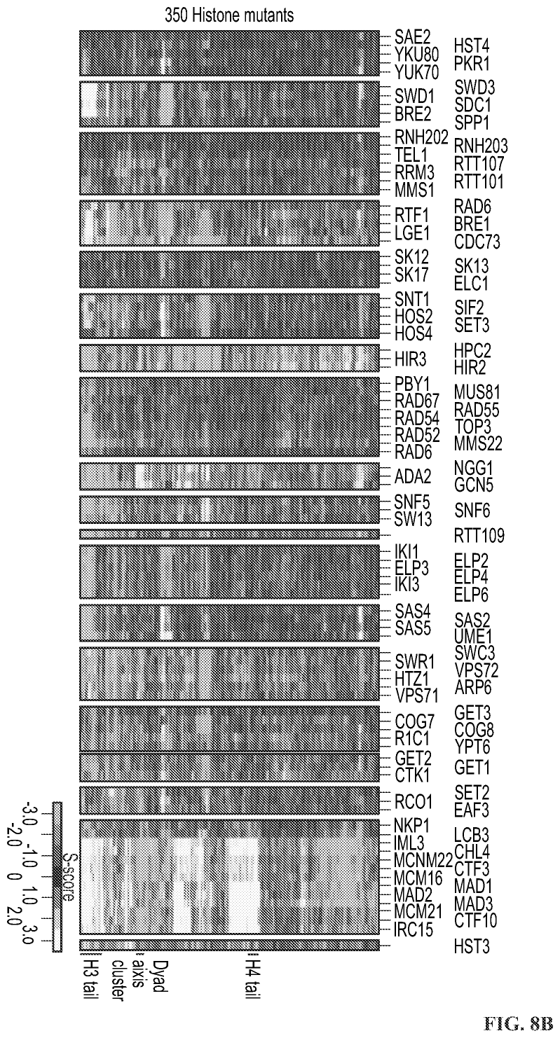

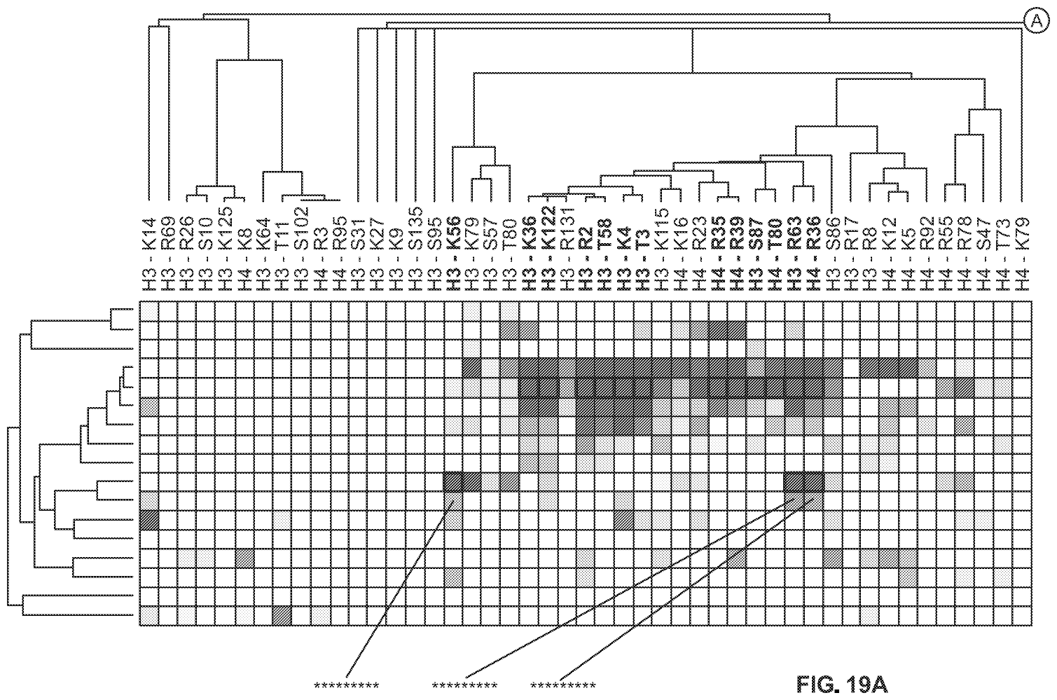

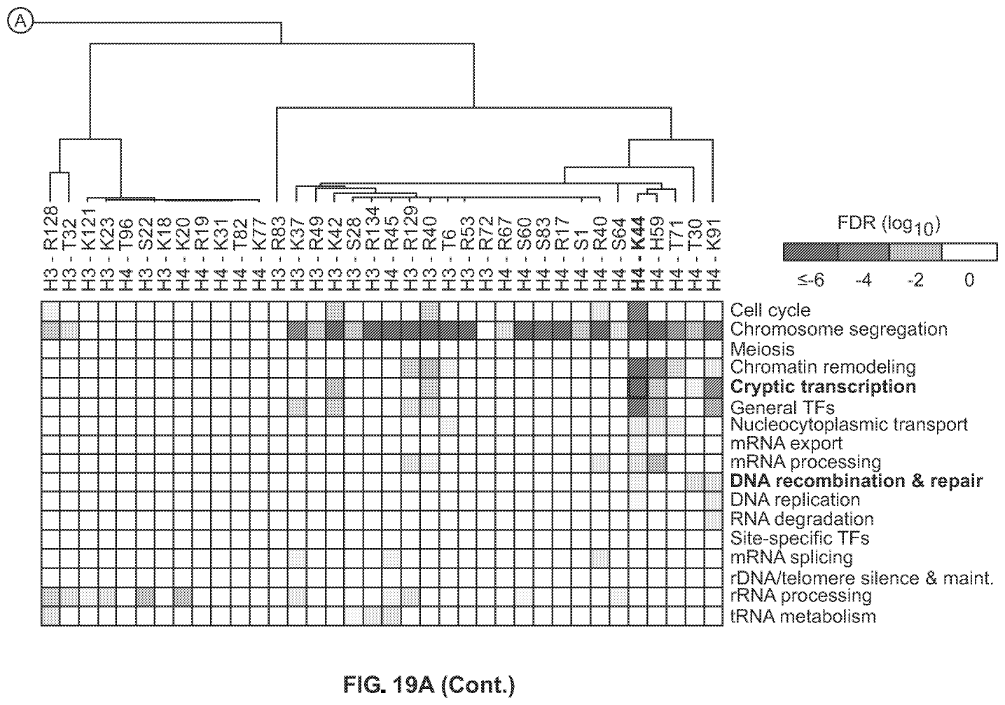

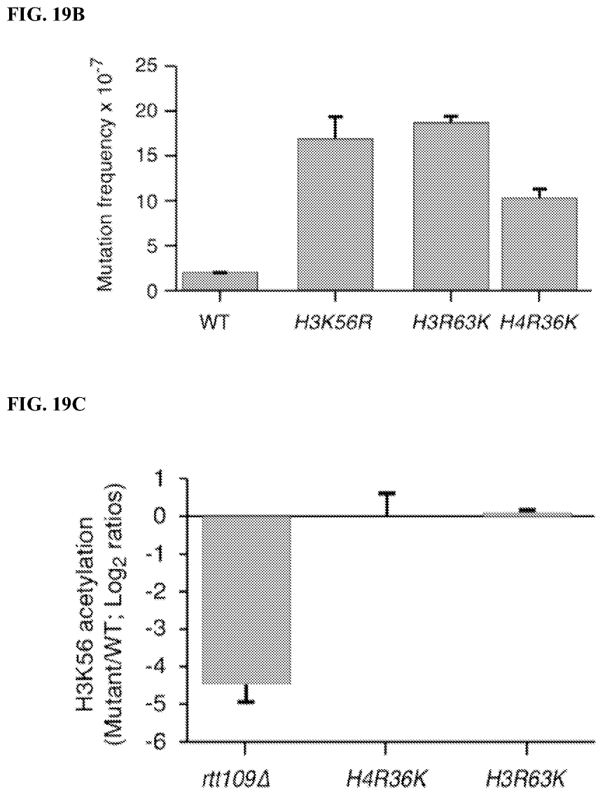

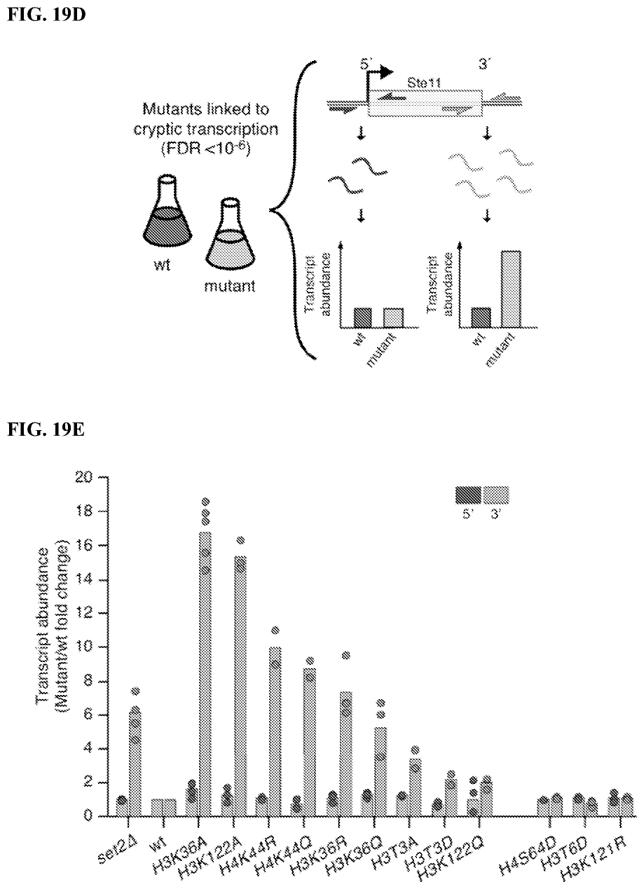

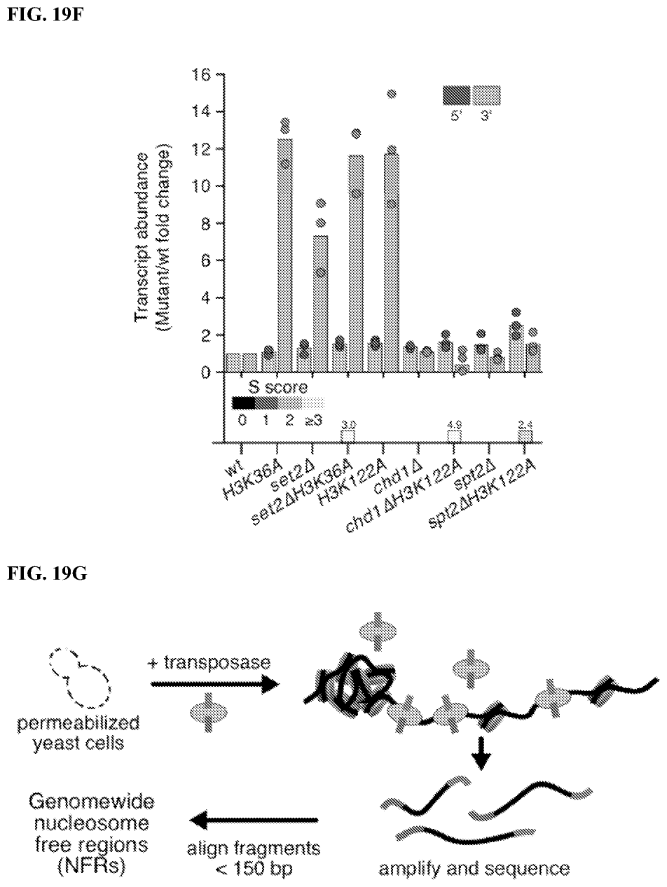

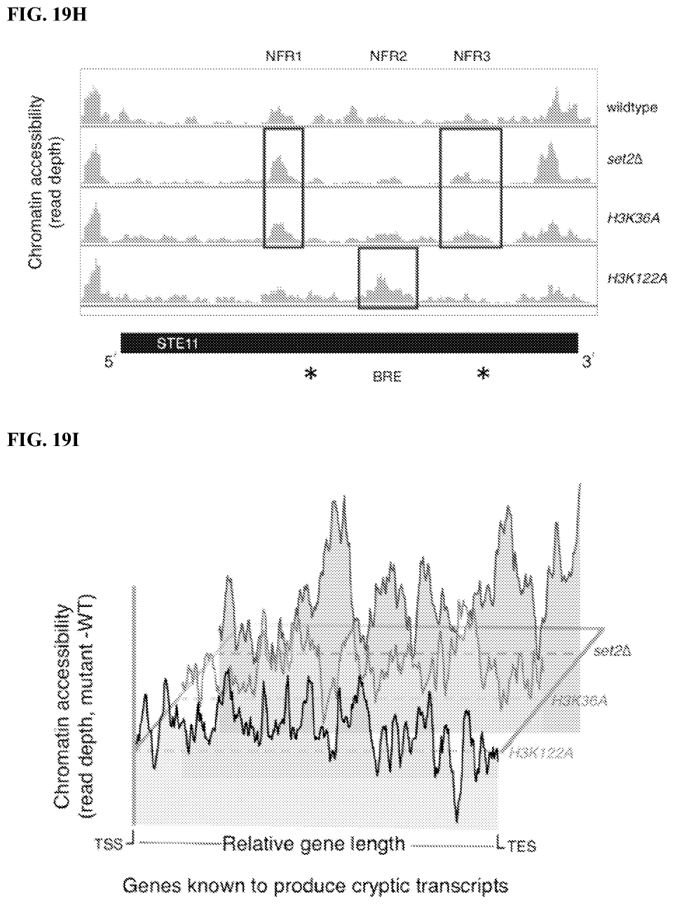

The pE-MAP was hierarchically clustered in both histone mutant and gene deletion dimensions using Cluster 3.0 (103) and displayed using Java Treeview (74) (FIG. 8). Images highlighting histone residues in context of the nucleosome structure (PDB: 1ID3 or its modified version) were created using ChimeraX (FIG. 8A, 4C, 7D, 13) (41). Reads mapping to nucleosome free regions as determined by ATACseq were visualized using the Integrated Genomics Viewer (FIG. 19H) (104).

Distance of Clustered pE-MAP Profiles

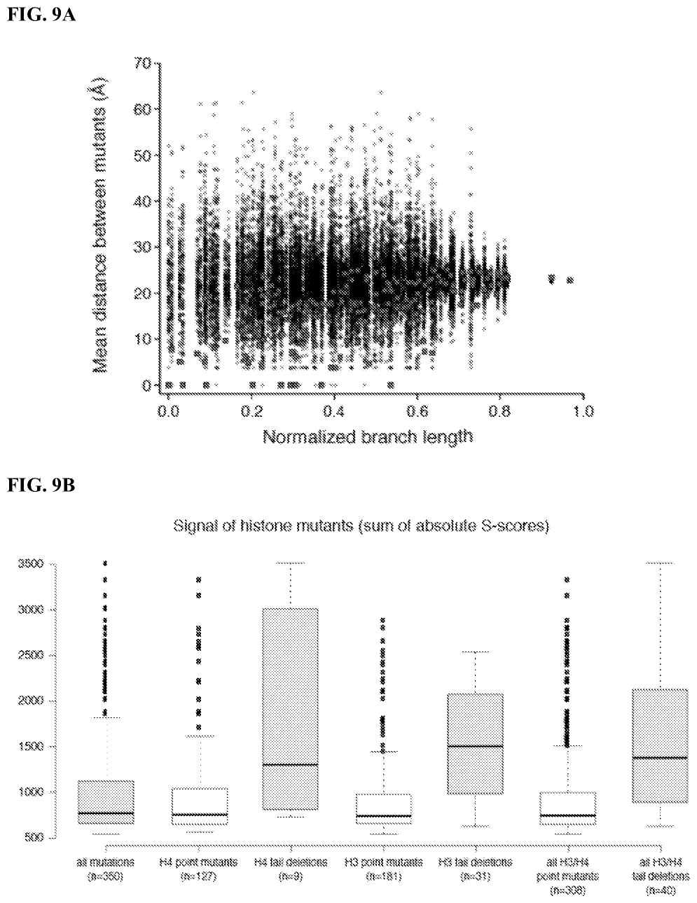

First, all histone alleles affecting residues not included in the structural reference (PDB: 1ID3, H3A1-H3K37 and H4S1-H4R17) were removed and the remaining data (n=222) clustered hierarchically using Cluster 3.0 (103). For each node of the clustergram, the mean distance among member residues was calculated and plotted vs the normalized branch length (where the first node is set to branch length=0 and the last node to branch length=1) of the respective node (FIG. 9A, red dots, and random distribution plotted in black).

Correlations

All correlations are Pearson correlation coefficients, unless otherwise noted. Genetic interaction correlations are based on the complete genetic interaction profiles.

Generation of the Correlation Map

Pearson correlation coefficients were computed for each of the 350 H3/H4 mutants against all genes/alleles (rows) in a merged map of previously published genetic interaction data (Dataset S4 from (38)). If the overlap between a histone mutant and a S-score vector from the merged map was <150 scores, the resulting correlation was not considered (i.e. replaced by “NaN”). Pearson correlation coefficients were chosen over MIC for this analysis because we found Pearson correlation more robust than MIC when many missing values were present.

Structural Mapping of Genetic Interactions stEMAP app

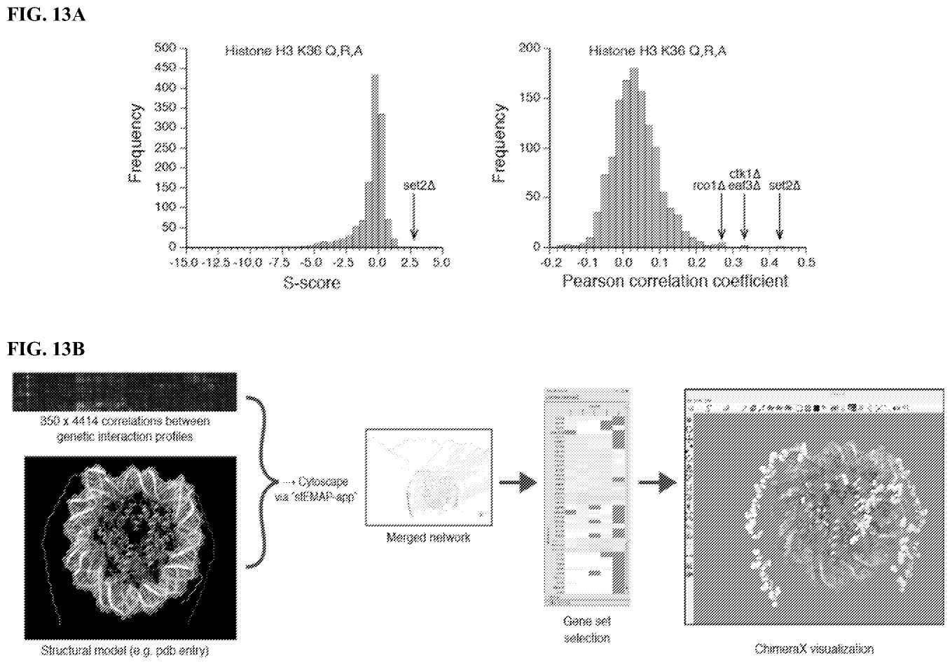

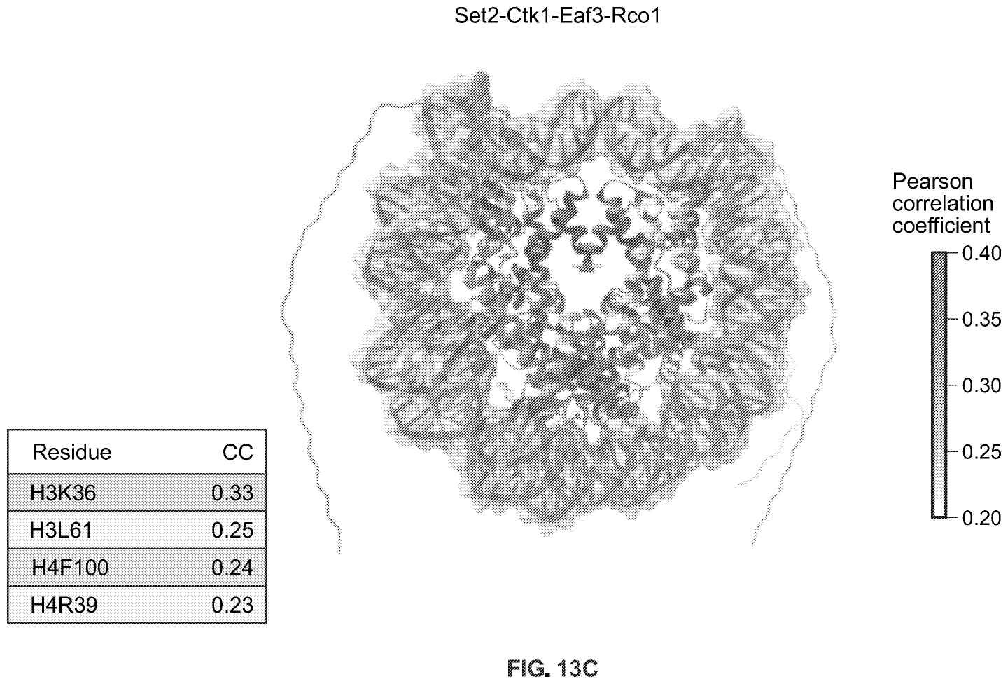

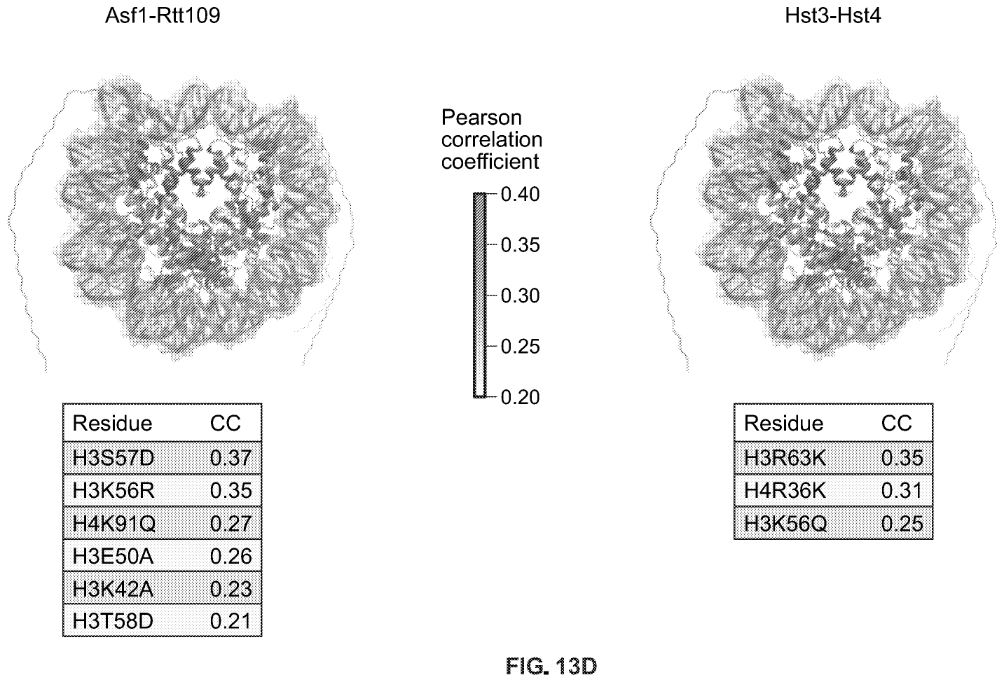

The hierarchically clustered pE-MAP data was imported into Cytoscape (40) creating an initial network and then linked to a modified version of the nucleosome structure 1ID3 using the stEMAP app, developed to facilitate interactive exploration of the pE-MAP. The original nucleosome structure was modified by adding the N-terminal disordered regions of histone H3 and H4 and manually positioning them for clarity. The linking proceeds as follows: first, the structure is opened in ChimeraX (41) by structureVizX (105) and positioned in response to commands from the stEMAP app. Then, a residue interaction network (RIN) is created by the structureVizX app where nodes are positioned to reflect the nucleosome structure through the help of the RINalyzer app (106). Finally, the RIN network and the network created by the original cluster files are merged, and edges are drawn between genes and residues with significant interactions (FIG. 13B). All of the preceding steps happen automatically through the stEMAP app interface, which takes as input any given PDB file and a short user-defined JSON configuration file defining significance thresholds (here: CCs>0.2), colors of edges (here: color-gradient from white to red for positive CC values), and display style of the structure in ChimeraX.

Selection of individual genes triggers the interacting residues to be selected and, in the ChimeraX window those residues are shown as space-filling atoms, which are colored according to the edge colors. When multiple genes are selected (e.g. genes belonging to the same complex), there might be multiple edges connecting an individual residue. In this case, the color reflects the significance and consistency of the interactions (see below). To assist in interpretation and interactive exploration of complex data sets i) colors are quantized into 10 bins, 5 positive and 5 negative, ii) a heatmap is presented that shows only the values for the selected genes and their interacting residues, iii) sets of genes belonging to a complex can be selected using the setsApp (107) and iv) a slider provides a filter to restrict the selection to only those mutations with a minimum number of interactions.

To determine if a gene set is connected to a given residue, the stEMAP app calculates the median CC across all genes of the gene set against all different mutations at that residue. If this median CC is above the threshold of 0.2 (defined in the JSON file), the respective residue is colored according to the median. To instead determine if a gene set is connected to an individual mutation, the same method is used, except the median CC is now calculated across all genes of the gene set against the single given mutation (instead of all mutations of the residue).

ROC Curves

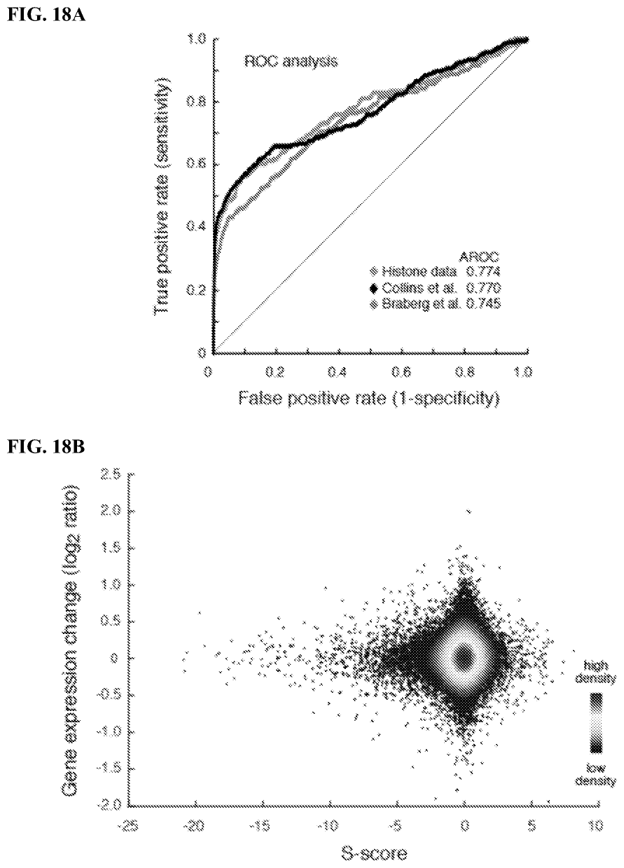

Only library deletion mutants that exist in both this study and the two previously published E-MAP data sets, Braberg et al. (75) and Collins et al. (76), were included (n=389) in this analysis. Based on their pE-MAP profiles, Pearson correlation coefficients were calculated for all pairwise combinations of these 389 deletion mutants. In order to determine the power of these correlations to predict physical interactions between encoded proteins an ROC curve was computed, where a physical interaction between proteins was defined if their PE score was larger than 2 in (52). From the Collins et al. E-MAP, query strain profiles with more missing data than the sparsest histone mutant were removed, as were query mutants that also existed in the library mutant set. Since the Braberg et al. pE-MAP only includes 53 query mutants (rows), we used subsets of 53 query mutants each for the histone and Collins et al. E-MAPs when generating their ROC curves, to make all three systems comparable. To this end, for the Collins et al. E-MAP and histone pE-MAP, 53 query mutant profiles were randomly selected 1,000 times, and an ROC curve was generated for each run. The median AROCs and corresponding ROC curves are reported together with the ROC curve of the pE-MAP from Braberg et al. in FIG. 18A.

RNA-seq Expression Analysis

10 ml of overnight cultures of 29 histone mutant strains (Table 6) were harvested in mid-log phase (OD600≈1.0) and washed with DEPC-ddH2O. RNA was extracted with hot acidic phenol as described previously (108). RNA-seq libraries were generated using the QuantSeq 3′ mRNA-Seq Library Prep Kit FWD for Illumina (Lexogen) and sequenced on Illumina HiSeq 400 sequencer. Single-end, 50 base reads were sequenced using an Illumina HiSeq 4000 sequencer. Reads were filtered for quality and aligned to the yeast genome using tophat (109). Non-unique reads and reads mapping to ribosomal RNA were removed prior to analysis. Transcript counts were extracted using htseq-count (110) and differential expression was measured using the Dseq2 package in R (111). Sequencing reads will be uploaded to a public database prior to submission.

Identification of Functional Links Between H3/H4 Mutants and Biological Processes

The correlation map was used as the basis for this analysis. Biological process definitions for genes in nuclear processes were assigned manually based on literature and annotations from previous genetic interaction maps (76, 112, 113). To identify H3/H4 residue-process pairs that were significantly correlated, we used a one-sided Mann-Whitney U test to compare the correlations between the mutants of each H3/H4 residue and the members of each process to (i) the correlations between the same H3/H4 mutants and all genes not in that process, and to (ii) the correlations between the same process and all other H3/H4 mutants. The highest p-value of the comparison to (i) or (ii) was recorded. False discovery rates (FDR) were computed using the method of Benjamini and Hochberg (114), and are reported in Table 7.

Spontaneous Mutation Frequency

Cells were grown to saturation and then plated on YEPD and 5-FOA supplemented media. Mutants growing on 5-FOA were counted only after confirming that colonies growing on YEPD for all the strains under study were of equal size. The assay was repeated three times independently and the average of all the three sets are plotted (Table 8).

MS Quantification of H3K56ac Levels

(1) Generation of Targeted Proteomics Assay

Peptide mixtures (obtained from ThermoFisher) were analyzed by LC-MS/MS on a Thermo Scientific Orbitrap Fusion mass spectrometry system equipped with a Proxeon Easy nLC 1200 ultra high-pressure liquid chromatography and autosampler system. Samples were injected onto a C18 column (25 cm×75 um I.D. packed with ReproSil Pur C18 AQ 1.9 μm particles) in 0.1% formic acid and then separated with a 60 min gradient from 5% to 40% Buffer B (90% ACN/10% water/0.1% formic acid) at a flow rate of 300 nl/min. The mass spectrometer collected data in a data-dependent fashion, collecting one full scan in the Orbitrap followed by collision-induced dissociation MS/MS scans in the dual linear ion trap for the 20 most intense peaks from the full scan. Dynamic exclusion was enabled for 30 seconds with a repeat count of 1. Charge state screening was employed to reject analysis of singly charged species or species for which a charge could not be assigned. The raw data was matched to protein sequences by the MaxQuant algorithm (version 1.5.2.8) (115). Data were searched against a database containing SwissProt Human sequences concatenated to a decoy database where each sequence was randomized in order to estimate the false discovery rate (FDR). Variable modifications were allowed for methionine oxidation and protein N-terminus acetylation and lysine acetylation. A fixed modification was indicated for cysteine carbamidomethylation. Full trypsin specificity was required. The first search was performed with a mass accuracy of +/−20 parts per million and the main search was performed with a mass accuracy of +/−4.5 parts per million. A maximum of 5 modifications were allowed per peptide. A maximum of 2 missed cleavages were allowed. The maximum charge allowed was 7+. Individual peptide mass tolerances were allowed. For MS/MS matching, a mass tolerance of 0.8 Da was allowed and the top 8 peaks per 100 Da were analyzed. MS/MS matching was allowed for higher charge states, water and ammonia loss events. The data were filtered to obtain a peptide, protein, and site-level false discovery rate of 0.01. The minimum peptide length was 7 amino acids. Selected reaction monitoring (SRM) assays were generated for selected acetylation sites. SRM assay generation was performed using Skyline (116). For all targeted proteins, proteotypic peptides and optimal transitions for identification and quantification were selected based on a spectral library generated from the shotgun MS experiments. The Skyline spectral library was used to extract optimal coordinates for the SRM assays, e.g. peptide fragments and peptide retention times. For each peptide the 5 best SRM transitions were selected based on intensity and peak shape.

(2) Sample Preparation

Histone mutant cultures (wt, H3R63K and H4R36K) were harvested in mid-log phase (OD600≈1.0) using 250 mm ceramic filter funnel and 30 μm nitrocellulose membranes connected to high continuous wall suction. Yeast were removed from the nitrocellulose membrane and flash frozen for storage or used immediately for protein extraction. Per gram of yeast pellet 3 ml of Yeast-Protein Extract Reagent (Y-PER; ThermoFisher Scientific) with added protease inhibitors (cOmplete™ Sigma-Aldrich, 1 tablet/50 mL), phosphatase inhibitors (PhosSTOP™ Sigma-Aldrich; 1 tablet/50 mL), histone deacetylase inhibitors (sodium butyrate 100 mM and nicotinamide 100 mM), and beta-mercaptoethanol (15 mM) were added. The suspension was mixed on a gyrator at 4° C. for 30 minutes and centrifuged. Pellets were resuspended in fresh Y-PER medium, and extraction was repeated two additional times for a total of three extractions. Pellets were sequentially washed twice with 3 mL ddH2O per gram of yeast. Histone extraction was performed in presence of 2.5 ml of 8 M urea/0.4 N sulfuric acid per gram of yeast protein pellets, incubated for 1 hour, centrifuged, and supernatants collected. Proteins were precipitated using a methanol-chloroform precipitation as previously described (117). Extracted proteins were trypsin digested, desalted and acetylated peptides were enriched as previously described (118).

(3) Targeted MS Data Acquisition and Analysis

Digested peptide mixtures were analyzed by LC-SRM on a Thermo Scientific TSQ Quantiva MS system equipped with a Proxeon Easy nLC 1200 ultra high-pressure liquid chromatography and autosampler system. Samples were injected onto a C18 column (25 cm×75 um I.D. packed with ReproSil Pur C18 AQ 1.9 um particles) in 0.1% formic acid and then separated with a 60 min gradient from 5% to 40% Buffer B (90% ACN/10% water/0.1% formic acid) at a flow rate of 300 nl/min. SRM acquisition was performed operating Q1 and Q3 at 0.7 unit mass resolution. For each peptide the best 5 transitions were monitored in a scheduled fashion with a retention time window of 5 min and a cycle time fixed to 2 sec. Argon was used as the collision gas at a nominal pressure of 1.5 mTorr. Collision energies were calculated by, CE=0.0348*(m/z)+0.4551 and CE=0.0271*(m/z)+1.5910 (CE, collision energy and m/z, mass to charge ratio) for doubly and triply charged precursor ions, respectively. SRM data was processed using Skyline (116). Protein significance analysis was performed using MSstats (119). Normalization of the intensities across samples was performed using the acetylated peptides H3K9_H3K14, H3K23 and H3K14 as global standards, which did not show any change across the mutants. Log 2-fold changes were calculated from three independent runs and plotted (FIG. 19C, Table 8).

Cryptic Transcription—Quantitative Realtime Polymerase Chain Reaction

Total RNA was extracted from 10 OD₆₀₀ units of mid-log phase cells (wt and respective mutant strains) using hot acid phenol-chloroform extraction method as described. 10 μg of total RNA was DNAse I treated (Promega) followed by purification using an RNeasy minikit (Qiagen). 1 μg of DNAse I treated total RNA was used to synthesize cDNA using SuperScript III first strand synthesis system (Life Technologies) and random hexamer primers. cDNA was diluted 1: 25 prior to amplification by PCR using primers designed for the 5′ and the 3′ ends of the STE11 gene. Quantitative realtime polymerase chain reaction (qRT-PCR) was performed using SYBR green (Biorad) as described previously. Relative change in the transcript levels were estimated using the ΔΔCₜ method described in (120) and were normalized to ACT1 transcript (Table 9). Primers sequences are available upon request.

Western Blotting

Whole yeast cell lysates were prepared using TCA lysis as described previously (121). Lysates were subjected to immunoblotting according to standard procedures and protein were detected using ECL Prime (Amersham Biosciences). Membranes were probed with αH3K36me-antibody was purchased from (Abcam, Catalog #9050), αGAPDH used for loading control was purchased from Sigma (Catalog #A9521).

Assay for Transposase-Accessible Chromatin Using Sequencing

Yeast cells (2.5×10⁶) were grown to mid-log phase, pelleted, washed with SB-buffer (1.4 M Sorbitol, 40 mM HEPES-KOH pH 7.5, 0.5 mM MgCl2), resuspended in 200 μl SB buffer+10 mM DTT with 10 μl of 10 mg/ml 100T zymolyase (MP Biomedicals) solution and incubated for 5 min at 30° C. Spheroblasted cells were washed with SB-buffer and incubated for 15 min at 37° C. in 25 μl transposase solution (12.5 μl 2× TD buffer, 1.25 μl Nextera enzyme, 11.25 μl water). DNA was purified (Qiagen MinElute DNA Purification Kit), amplified and barcoded by PCR. Purified PCR-products were sequenced using an Illumina HiSeq 4000 sequencer. Sequence reads were trimmed, aligned to the genome of S. cerevisiae (version SacCer3 from hgdownload.cse.ucsc.edu/downloads.html) and reads with a length <100 bp removed. Replicates belonging to an allele (wildtype, H3K36A, H3K122A, set2Δ) were merged and normalized to the smallest read number. For visualization of STE11 read coverage using the IGV genome browser (104) (FIG. 19H), each track was scaled linearly so that the largest peak in the displayed window is the same height for all tracks. Count files were generated with “featureCounts v1.5.3” (122).

Gene body plots (FIG. 19I) were generated as follows. First, counts from genes reported to be targets of cryptic transcription (n=11; FLO8, AVO1, LCB5, SMC3, SPB4, APM2, DDC1, SYF1, OMS1, PUS4, STE11), as well as counts 400 bp up- and downstream of the respective gene bodies, were extracted. Then, up- and downstream regions were split into 50 bins of equal size (8 bp), whereas the gene body was split into 300 equal bins, resulting in 400 bins for each gene in each tested strain (wildtype, set2Δ, H3K36A and H3K122A). Next, for each of the 400 bins the average for the 11 target genes was calculated. Each mutant allele was then scaled linearly so that the first bin (i.e. 400 bp upstream of the gene body start) was equal to that of wildtype, and finally the wildtype counts were subtracted from the mutant counts for each bin.

Data and Materials Availability

IMP modelling scripts, input data, and output results are available at http://integrativemodeling.org/systems/pemap. IMP is an open-source program, available at http://integrativemodeling.org. The yeast RNAPII pE-MAP is available in Table 2 in a previous study (14). The clustered pE-MAP and correlation maps can be visualized using the Java Treeview app (74), available at http://jtreeview.sourceforge.net/. The stEMAP app source is publicly available at https://github.com/RBVI/StEMAPApp and stEMAP will be available through the Cytoscape App store. stEMAP requires installation of Cytoscape and ChimeraX. Cytoscape is available at https://cytoscape.org/download.html and ChimeraX at https://www.rbvi.ucsf.edu/chimera/download.html. stEMAP relies on the Sets, RINalyzer, StructureVizX, clusterMaker2 and ctdReader apps, all available through the Cytoscape apps menu. The modified version of PDB 1ID3 (including the unstructured H3 and H4 tails for illustrative purposes) is disclosed herein. The bacterial RNAP point mutation data will be available upon publication of the corresponding manuscript (Anthony L. Shiver, Hendrik Osadnik, Jason M. Peters, Rachel A. Mooney, Robert Landick, Kerwyn Casey Huang, Carol A. Gross).

Results

A comprehensive pE-MAP of Histones H3 and H4

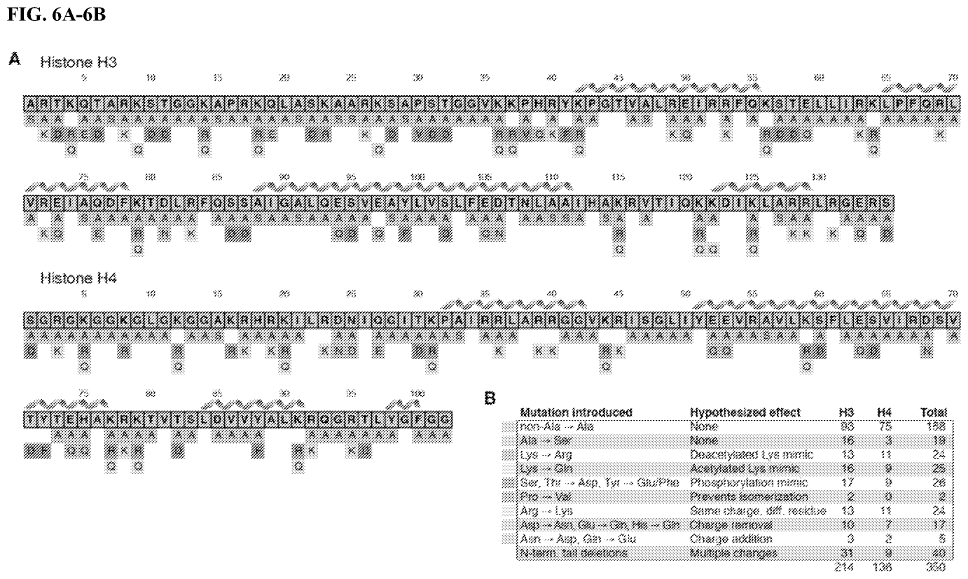

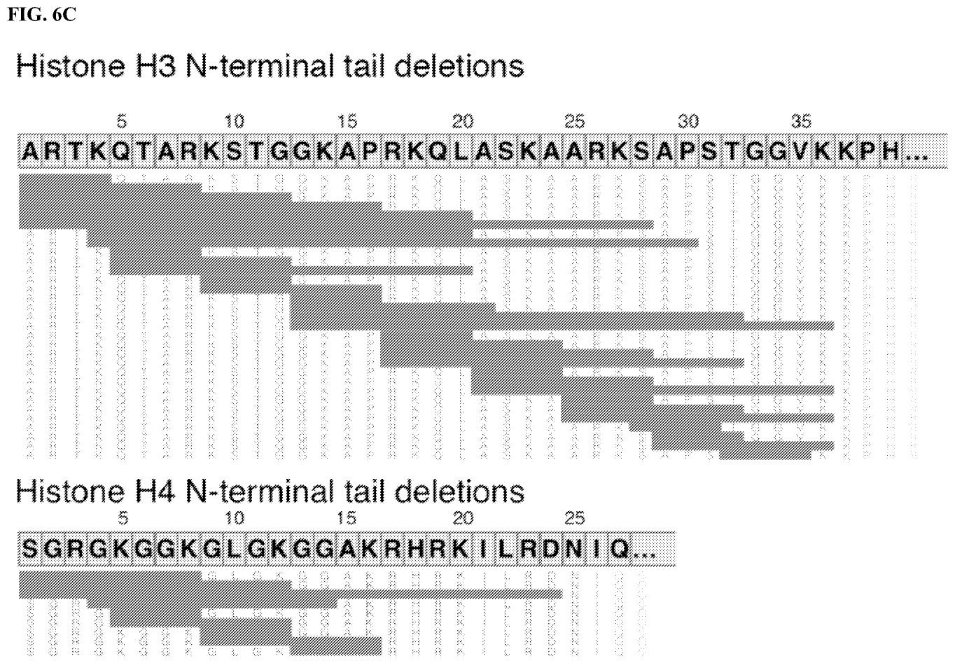

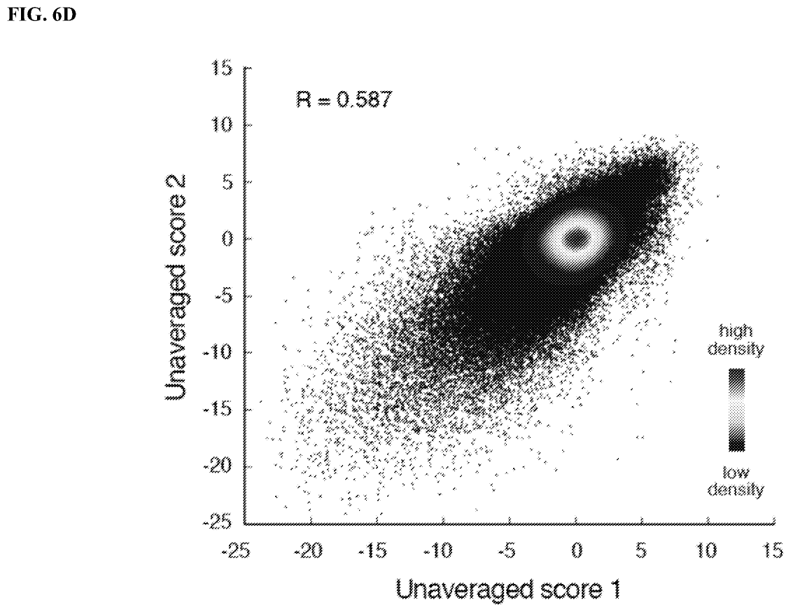

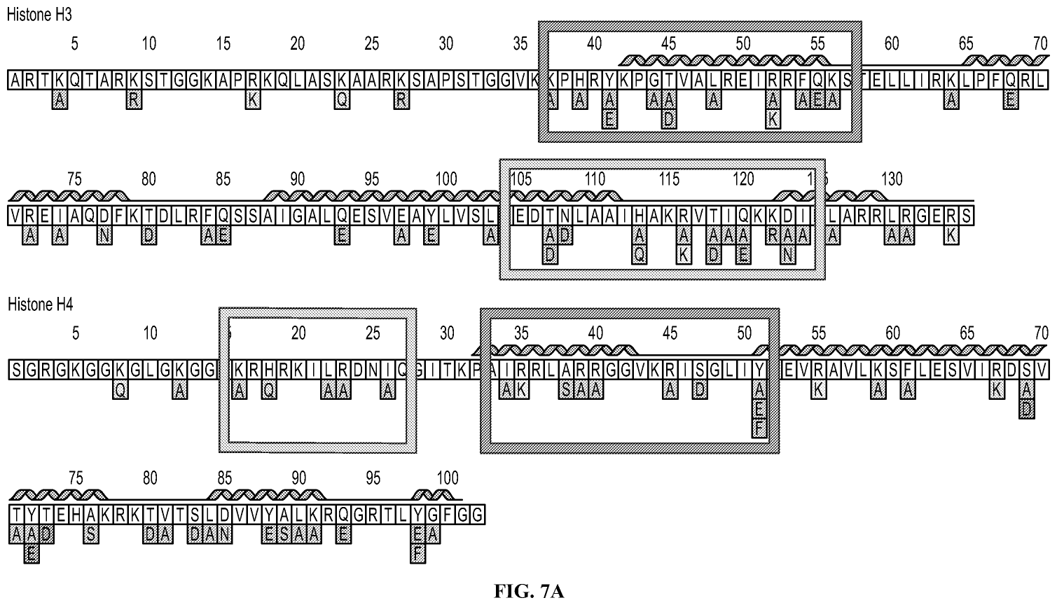

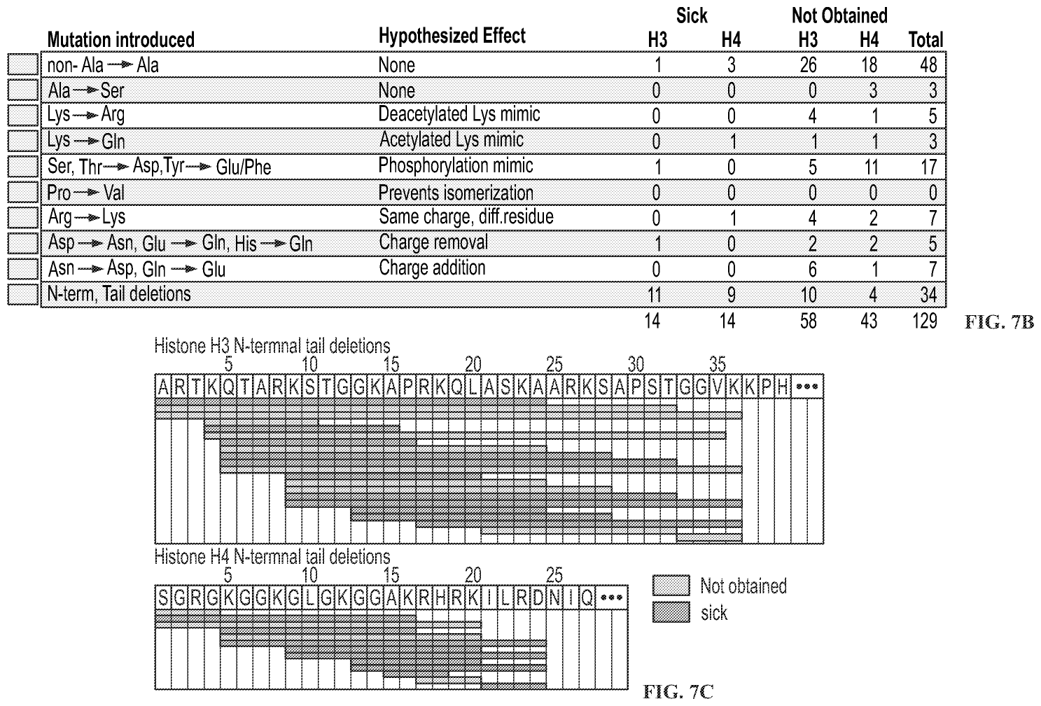

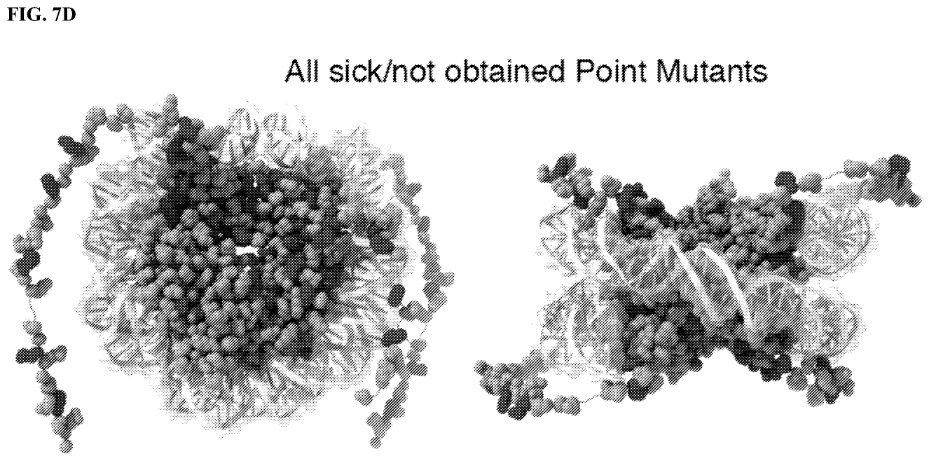

Histones are central to chromatin structure and dynamics, as they make up the core of the nucleosome, the fundamental repeating unit of chromatin. The state of the nucleosome is controlled by histone post-translational modifications (PTMs) (21), including acetylation, methylation, phosphorylation and ubiquitination, that help maintain and regulate chromatin structure and transcription. Our library of point mutations in the core histones H3 and H4 was designed to comprise a comprehensive alanine-scan, as well as context-specific mutations of modifiable residues (e.g. lysine/arginine or serine/threonine), such as charge removal/reversal and substitutions mimicking PTMs (22, 23). Partial deletions of the N-terminal tails of H3 and H4 were also included, as these regions play important and sometimes redundant roles in chromatin biology (24, 25). In budding yeast, histones H3 and H4 are expressed from two loci each, HHT1/HHT2 and HHF1/HHF2, respectively. To ensure preservation of the native expression levels, we engineered each strain to include identical point mutations in both relevant loci with separate selection markers (HYG^{R} and URA3). The histone mutants were crossed in a high-throughput fashion against a library of 1,370 gene deletions and hypomorphic alleles using our triple mutant selection strategy (26, 27) involving three different selectable markers (HYG^{R} and URA3 to select for both copies of the histone alleles and KAN^{R} for the knockout library strains) (FIG. 1C, Methods) (26). In total, we designed 479 histone mutants, of which 350 were amenable to pE-MAP analysis (FIG. 6, Table 1); the remaining 129 mutants were either lethal or exhibited very poor growth, rendering them inaccessible to pE-MAP analysis (FIG. 7). Genetic interactions were quantified by a statistical measure termed S-score (28), and the pE-MAP screen was carried out in 3 biological replicates, which exhibit a high reproducibility (FIG. 6D). The pE-MAP was clustered hierarchically along both dimensions (FIG. 1D) and effectively recapitulates known protein complex and pathway memberships. For example, the pE-MAP identified COMPASS (29, 30), Swr1-C(31), the Set2/Eaf3 pathway (32, 33), as well as clusters of genes linked to telomere maintenance and Golgi/ER traffic (FIG. 1E, FIG. 8). Furthermore, we found that H3 tail mutants displayed strong negative genetic interactions with genes involved in DDR (DNA damage/repair), whereas mutants in the H4 tail have strong positive genetic interactions with kinetochore components (FIG. 8). Mutations of histone residues in close proximity to each other (e.g. mutants of the H3 or H4 N-terminal tails) tend to cluster together, due to the high similarity of their phenotypic profiles (FIG. 8 and FIG. 9A). Overall, we find that histone tail deletion mutants give rise to stronger phenotypic profiles than the point mutants (FIG. 9B), reflecting the multiple residue perturbations and the importance of functional histone tails for cell homeostasis.

Phenotypic Profile Similarities are Correlated with Structural Proximity

Similarities between pairs of phenotypic profiles in the histone H3-H4 pE-MAP were quantified by the maximal information coefficient (MIC) (34, 35) (FIG. 1F, Methods, FIG. 10). The MIC values between pairs of phenotypic profiles do not linearly correlate with the distances between the mutated residues in the WT structure (Pearson correlation coefficient of −0.07), but are informative about an upper distance bound between the residues (FIG. 1G, FIG. 10C). The upper distance bound was obtained by binning the MIC values into 20 intervals and selecting the maximum distance spanned by any pair of residues in each bin, followed by fitting a logarithmic decay function to these maximum distances (Methods, FIG. 10C). The data show that a pair of proximal point mutations are more likely to have a high MIC value than a pair of distal point mutations. However, not all proximal mutations have a high MIC value: most pairs of phenotypic profiles, even those for residues that are less than 16 Å apart, are highly dissimilar (94% of pairs exhibit a MIC value <0.3). These observations justify converting the pE-MAP data into a Bayesian data likelihood that provides an upper bound on the distance spanned by the mutated residues (Methods, FIG. 1G (inset)). This Bayesian term objectively interprets the noise in the experimental data and allows us to quantify the uncertainty of the resulting structural models. Importantly, only the histone pE-MAP dataset was used to develop and parametrize the Bayesian term. The complete scoring function for evaluating any structural model also includes simple terms accounting for excluded volume and sequence connectivity, in addition to the Bayesian terms for all pairs of profiles in the pE-MAP with a MIC value above 0.3.

Spatial Restraints Derived from pE-MAP Data can be Used for Integrative Structure Determination

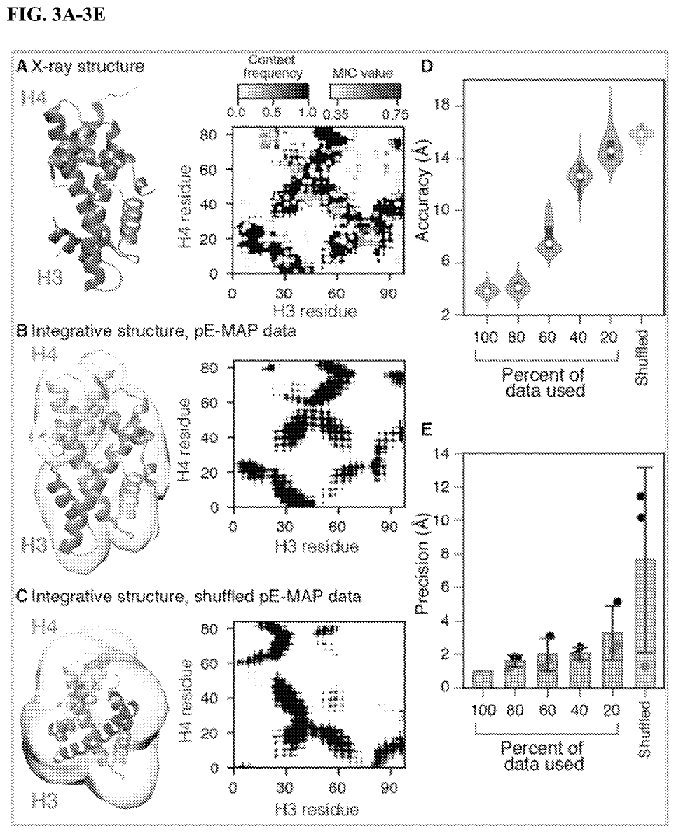

An ensemble of the H3-H4 dimer configurations that satisfy the input information (i.e. the model) was found by exhaustive Monte Carlo sampling guided by the scoring function, starting with random initial configurations of the rigid comparative models of the H3 and H4 subunits (FIG. 2). The resulting ensemble is accurate and precise, as demonstrated by the similarity between the X-ray (PDB: 1ID3, (36)) and model contact maps (FIG. 3A-B). Specifically, the accuracy is 3.8 Å (FIG. 3D); the accuracy is defined as the average Cα root-mean-square deviation (RMSD) between the X-ray structure and each of the structures in the ensemble. The precision is 1.0 Å (FIG. 3E); the precision is defined as the average RMSD between all solutions in the ensemble. As a control, we also computed a model from randomly shuffled MIC values. The resulting model (FIG. 3C) is incorrect (accuracy of 15.8 Å and incorrect contact map; FIG. 3C-D) and imprecise (7.6 Å; FIG. 3E). As another control, we computed a model by a state-of-the-art protein-protein docking method (37), resulting in a model with an inferior accuracy of 6.9 Å (FIG. 11). Finally, we also mapped the accuracy and precision of the model as a function of the fraction of the pE-MAP data used (Methods). As expected, the more pE-MAP data are used, the more accurate and precise is the model (FIG. 3D-E, Table 2). We estimate that, for systems in which the structures of the components are known, when performing systematic protein-wide residue substitutions, 35-40 mutations per protein are necessary to generate a good accuracy and precision model (FIG. 12, Methods). Importantly, this estimate is an upper bound on the number of mutations, and in many cases, the number might be reduced by specifically designing point mutations that target surface residues and/or residues known to be functionally important, and by choosing substitutions likely to give rise to functional perturbations. In conclusion, the outcome of these calculations indicates the utility of the pE-MAP data for integrative structure determination.

The pE-MAP Connects Individual Histone Residues and Regions to Other Associated Complexes

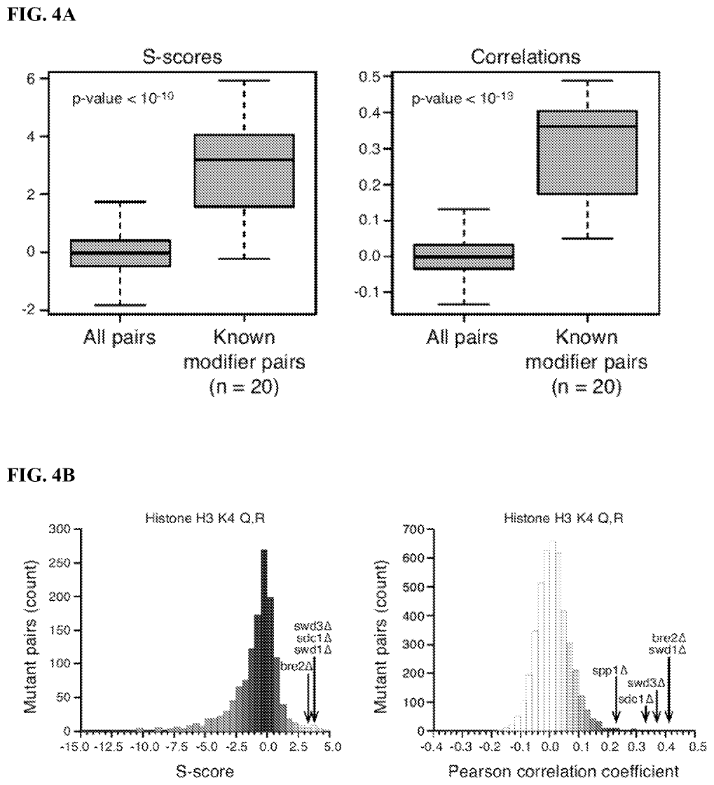

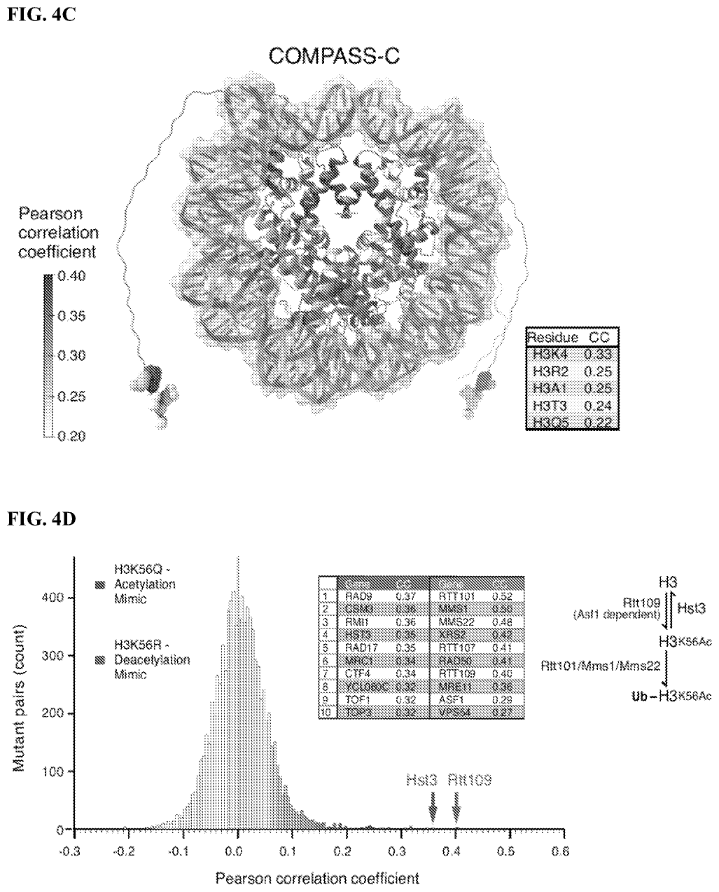

To examine whether the pE-MAP can identify interactions with complexes that are not stably associated with histones, we investigated the relationships between modifiable histone residues and their cognate enzymes (modifier pairs, Table 3). Interestingly, we observed a dramatic increase in S-scores within highly specific modifier pairs, as compared to the overall genetic interaction distribution (FIG. 4A, Methods). The positive S-scores reflect that a modifier and its target residue often are epistatic/suppressive because they function in the same pathway. To test if this pattern extends to phenotypic profile similarities, we integrated the histone pE-MAP into a merged map of previously collected genetic interaction data, covering a total of 4,414 gene deletions and hypomorphic alleles (38). We computed Pearson correlations for each histone mutant phenotypic profile across the merged map, generating a correlation map of 350 histone mutants against 4,414 whole gene perturbations (Methods). In agreement with the individual S-scores, the highly specific modifier pairs exhibit significantly higher phenotypic profile correlations than the overall map (FIG. 4A, Methods). These findings show that the pE-MAP can be used to pair specific residues to their respective modifiers, even though these are not stably associated with the histones. For example, when components of COMPASS, which methylates histone H3K4 (30, 39), are deleted (swd1Δ, swd3Δ, sdc1Δ, bre2Δ), we observe strong positive S-scores with the H3K4 mutants as well as high correlations of the overall genetic profiles (FIG. 4B). Similarly, members of the Set2 pathway (Set2, Eaf3, Rco1) (32, 33) rank very highly in the distributions of S-scores and correlations of their target residue, H3K36 (FIG. 13A).

To explore these relationships in a structural context, we developed a Cytoscape (40) App named stE-MAP (structure E-MAP) that interactively maps the genetic interactions of pE-MAP gene clusters onto the point mutated protein structure. stE-MAP connects Cytoscape to ChimeraX (41) and displays connections between a pre-defined set of genes and all mutated residues for which the underlying interactions pass user-defined criteria (FIG. 13B). We mapped the genetic connections between COMPASS and all histone residues with which it exhibits >0.2 median correlation (Methods). Only 5 residues pass this threshold, and the strongest connection is displayed by H3K4. The other 4 residues (H3K1-H3K3 and H3K5) are proximal and are thus likely to interfere with the interaction between COMPASS and H3K4 (FIG. 4C). This finding is particularly notable since these residues reside in the most distal region of the unstructured H3 N-terminal tail. Given that we do not have COMPASS point mutations in our dataset, we did not attempt to model this interaction. However, analysis of the MIC values associated with the H3 and H4 tails, and their relationship with the core domains, indicate that distance restraints for the histone tails could be derived from the pE-MAP data. Specifically, the MIC value distributions for the tail-core and tail-tail pairs of mutations are similar to that of the core-core mutations (FIG. 12C). This similarity indicates that we can derive distance restraints for the histone tails, thus, in principle supporting the feasibility of integrative structure modeling of disordered regions. We observed similar trends for other histone modifiers, including Set2-C, where H3K36 is the mostly highly correlated residue (FIG. 13C). Interestingly, we also found instances where different mutations of a single residue identify connections to different modifiers. For example, the phenotypic profile of the deacetylation mimic H3K56R is similar to that of deletion of RTT109, which encodes the H3K56 acetylase (Pearson correlation=0.4), whereas the acetylation mimic H3K56Q instead correlates with the profile generated from the deletion of the corresponding deacetylase, HST3 (Pearson correlation=0.35). H3K56R further correlates with asf1Δ, rtt101Δ, mms1Δ and mms22Δ, whose corresponding proteins play key roles in the H3K56 acetylation pathway and downstream H3 ubiquitylation (42, 43) (FIG. 4D). Accordingly, the stE-MAP app identified strong links between Hst3-Hst4 and H3K56Q, as well as Rtt109-Asf1 and H3K56R (FIG. 13D). While we find that it is often informative to group different mutations of the same residue together, these examples highlight the potential of these maps for deeper mechanistic insights where required.

The Integrative Structure Determination Approach is Transferable to Other Complexes

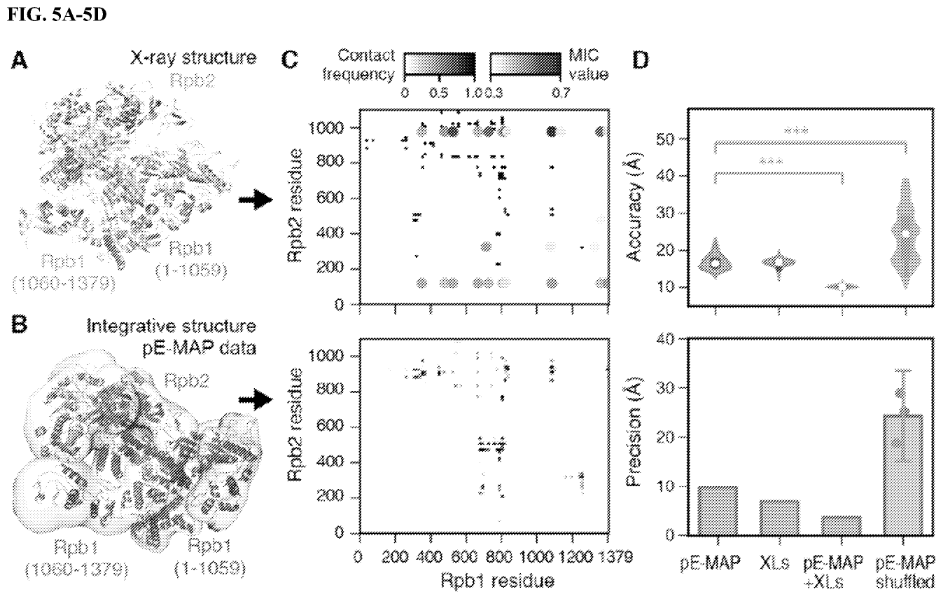

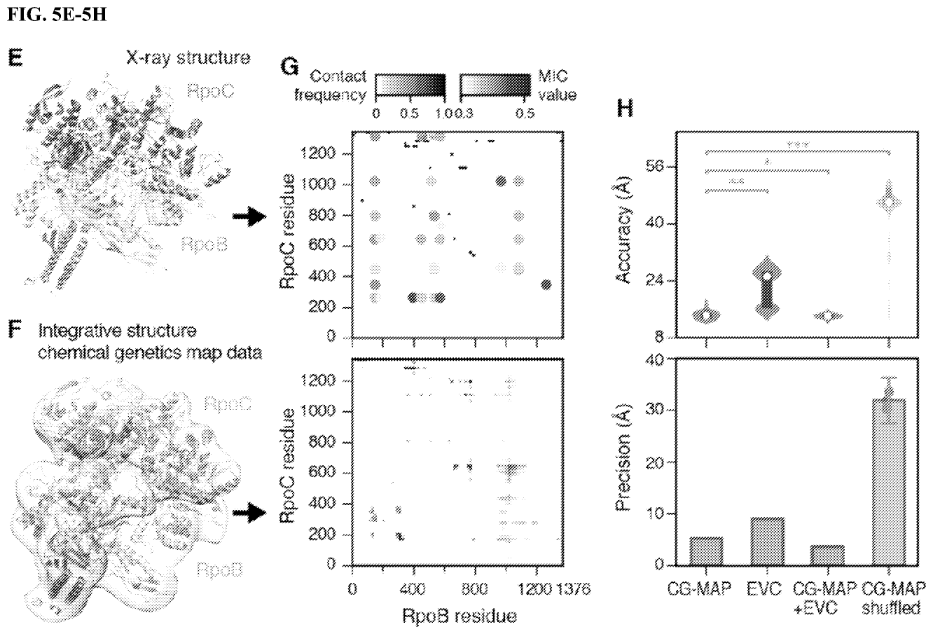

To examine whether genetic interaction mapping can be used to determine the structure of other complexes, we examined a pE-MAP of RNA polymerase II (RNAPII) in budding yeast. Interestingly, the association between the MIC values and distance upper bound is also apparent in this dataset (FIG. 14A, 14C), even though the protein sizes and mutational coverage of the polymerase system (>1,200 residues and 1-2%, respectively) are vastly different from those of the histones (<140 residues and 85-90%). These observations suggest that our parameterization of the pE-MAP spatial restraint based on the histone data may be generally applicable. To evaluate this expectation directly, we next modeled RNAPII using the Bayesian likelihood/prior parametrization based on the histone pE-MAP. The configuration of subunits Rpb1 and Rpb2 was modeled using a pE-MAP of 53 single point mutants crossed against a library of 1,200 gene deletions. To illustrate the modeling of higher order complexes, we divided the Rpb1 subunit into two domains, thereby representing the system with three rigid-bodies (Methods). We obtained a model with an accuracy of 16.8 Å and precision of 9.8 Å (FIG. 5A-D, Table 4). This positive result illustrates the generality of the pE-MAP based spatial restraints.

To further assess the utility of pE-MAP data for structure determination, we compared the RNAPII model obtained using pE-MAP to a model using 22 previously published chemical cross-links (XLs) (44). Cross-linking is widely used for integrative structure determination of macromolecular assemblies (2, 8). Interestingly, a model of yeast RNAPII based on the pE-MAP data is as accurate as that based on the cross-links (16.8 Å and 16.7 Å, respectively; FIG. 5D). Moreover, the accuracy of the model improves if both datasets are used simultaneously (7.4 Å; FIG. 5D), indicating complementarity between the two types of data and demonstrating a premise of integrative structure determination (FIG. 2). While a cross-link between two residues may provide more direct structural information than the corresponding pE-MAP pair, the number of possible cross-links is limited by the number of proximal lysine pairs, whereas the number of pE-MAP pairs grows quadratically with every additional point mutation introduced. Therefore, the larger number of less precise pE-MAP restraints can lead to a more accurate model than a smaller number of more precise cross-links.

The Integrative Structure Determination Approach is Transferable to Other Types of Phenotypic Profiles

To test the applicability of our approach to other types of phenotypic profiles, we turned to a chemical genetics mini-array profile (CG-MAP) of 44 bacterial RNAP point mutations exposed to 83 different environmental stresses (e.g. chemical perturbations, temperature stress, and pH change) (Shiver et al., in preparation). We observe an association between MIC values and distance upper bound, similar to that of the pE-MAP datasets (FIG. 14A, 14D, Methods). We modeled the structure of subunits RpoB and RpoC of the bacterial RNAP with an accuracy of 14.3 Å and precision of 5.3 Å (FIG. 5E-H, Table 5). This result suggests that maps with relatively small numbers of orthogonal phenotypes per point mutation can be used to accurately predict the architecture of macromolecular assemblies. Considering that constructing large gene deletion libraries and crossing them against point mutations can be laborious, environmental phenotypic profiles may be a more efficient alternative for generating spatial restraints for integrative structure determination than genetic phenotypic profiles.

Spatial Restraints Derived from pE-MAP Data are Comparable to Other Commonly Used Data Types

Co-evolution information can also be used for prediction of protein assembly structures (17, 45, 46). However, the success of such modeling is heavily dependent on the number of sequences in the input sequence alignments and the ability to discriminate interacting from non-interacting homologs in genomes with multiple paralogs (47). Using the RaptorX protein complex contact prediction server (48, 49), we predicted the interfacial contacts between RpoB and RpoC of the bacterial RNAP; the numbers of homologous sequences were insufficient for the yeast histones and RNAPII (Methods). Importantly, RaptorX is based on a combination of co-evolution analysis and a deep-learning algorithm that reduces the requirement for sequence homologs and improve accuracy (50). Other commonly used co-evolution methods (17, 45) did not identify any interfacial contacts. Similar to the pE-MAP and CG-MAP datasets, we observe a negative statistical association between the residue pair coupling strength and distance upper bound (FIG. 15). To mimic the pE-MAP restraint, we converted the top coupling strengths into distance upper bound restraints (Methods). The model ensemble computed from co-evolution derived restraints includes two different sets of configurations (accuracy of 22.6±5.1 Å; model precision of 9.0 Å; FIG. 5H). Only a fraction of the bacterial RNAP structures computed using co-evolution derived restraints are as accurate as those computed using the CG-MAP restraints. The model precision, though not the accuracy of the model, improves if both types of restraints are combined (accuracy of 14.1 Å; model precision of 3.7 Å; FIG. 5H).

Histone pE-MAP Quality Control, Signal and Content

It has been shown that a pE-MAP can be used to predict protein-protein interactions (PPIs), by comparing the genetic interaction (GI) patterns between pairs of deletion mutants across all the point mutants (75, 76). On a global level, this is only possible if the point mutant set perturbs a broad group of processes and exhibits genetic interactions with the many different deletion mutants that encode the interacting proteins. Since the histone mutant collection perturbs only two genes (i.e. H3 and H4), we set out to investigate whether the resulting genetic interaction profiles are sufficient to be predictive of PPIs among the 1,370 deletion mutants. We thus generated a receiver operating characteristic (ROC) curve, and find that the histone pE-MAP performs similarly to previous E-MAPs affecting more genes (75, 76) (FIG. 18A, Methods). This finding indicates that the combined set of histone point mutants affects a broad set of cellular processes, reflecting the multifunctional nature of histones H3 and H4 and their central role in controlling the global genetic environment of cells.

To gain insight into the regulatory hierarchy that drives the widespread functional effects of histone perturbations, we set out to determine if there was a relationship between genetic interactions and gene expression changes. To this end, we determined the genome-wide gene expression levels for 29 representative histone mutants using RNA-Seq and found no correlation between the expression change of a gene resulting from a given histone mutation and the corresponding S-score (FIG. 18B, Table 6). This indicates that observed genetic interactions between histone mutations and deletion mutants are due to complex regulatory patterns, rather than the histone mutation directly modulating the expression of the interacting gene.

To generate a systematic functional mapping of histone residues, we devised a curated annotation of all genes in our correlation map relevant to nuclear function (Table 7), and built a connectivity map between modifiable histone residues and processes using gene set enrichment analysis (GSEA) (FIG. 19A, Methods, Table 7). We focused on the most significant connections with a false discovery rate (FDR)<10⁻⁶, and observed both known and novel connections between histone residues and nuclear pathways. For example, “DNA recombination & repair” is connected to 4 residues, and two of these, H3K56 and H3K79, have already been shown to play key roles in yeast's DNA repair (123-127). To investigate the involvement of the other two residues (H3R63 and H4R36) in DNA repair, we measured the spontaneous mutation frequencies of these R->K strains compared to wt (Methods). Indeed, both H3R63K and H4R36K exhibit strongly increased mutation frequencies at the URA3 locus compared to wt, at a comparable level to H3K56R (FIG. 19B, Table 8). Given the central role of H3K56 acetylation in DNA repair, we tested whether H4R36K and H3R63K alter H3K56ac-levels using quantitative mass spectrometry, but found no effect (FIG. 19C, Table 8). These findings indicate that H3R63 and H4R36 play roles in DNA repair via a mechanism independent of H3K56 acetylation.

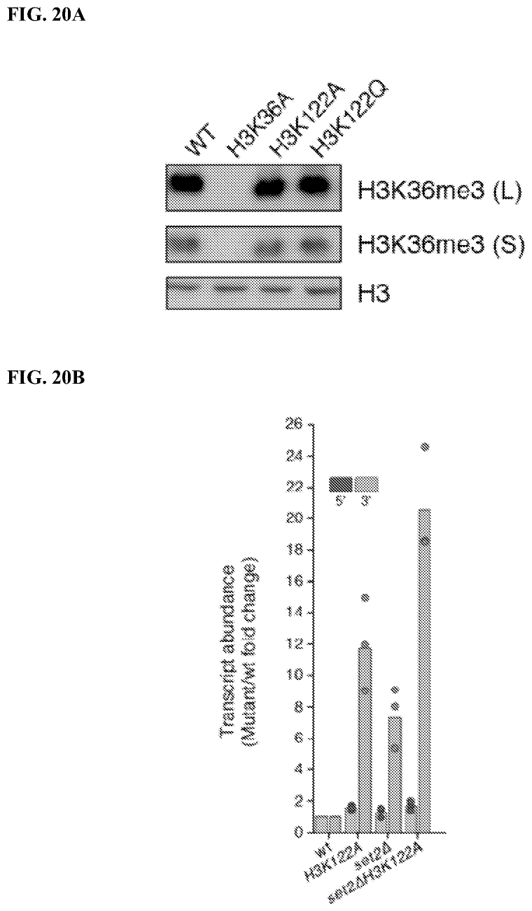

Interestingly, the GSEA map also identified a group of 13 residues connected to cryptic transcription (FIG. 19A). Our screen included 24 different mutants of these residues, and we tested their involvement in cryptic transcription by quantifying the abundance of transcripts at the 5′ and 3′ end of the STE11 gene, known to produce cryptic transcripts (32, 128), using qPCR (FIG. 19D). In total, 16 mutations, distributed among 10 residues, increase 3′ transcript abundance by >50% compared to wt (Table 9), and 9 mutants among 5 residues increase 3′ transcription over two-fold, without major changes in 5′ transcription (FIG. 19E). As expected, H3K36A, H3K36R, H3K36Q and set2Δ increase 3′ transcript abundance strongly, as do mutations of H4K44, which is a residue known to affect cryptic transcription (129). Interestingly, H3K122A increases 3′-transcript abundance >15-fold. H3K122 is located at the dyad-axis interfacing with the DNA, and has been shown to function in nucleosome dynamics (130). H3K122A exhibits positive genetic interactions with deletion of the histone chaperone SPT2 (S-score=2.4) and the nucleosome remodeling factor CHD1 (S-score=4.9), which are both involved in cryptic transcription (131, 132). We found that deletion of either SPT2 or CHD1 suppresses the cryptic transcription phenotype observed in H3K122A to wt levels, even though spt2Δ or chd1Δ alone has no effect (FIG. 19F). The H3K122A-Chd1-Spt2 pathway is independent of Set2-K36me, as H3K122A has no effect on H3K36 methylation and set2Δ H3K122A exhibits a much greater 3′ transcript abundance change than either single mutant alone (FIG. 20).

H3K122 is at the histone-DNA interface (<5 Å), and its acetylation by Brd4 leads to nucleosome eviction in human cells (133). Nucleosome eviction, which is a requirement for transcription from cryptic promoters, can be facilitated by histone PTMs or amino acid substitutions that destabilize DNA-histone interactions (134). To determine the mechanism of cryptic transcription in H3K122A we used ATAC-seq (Assay for Transposase-Accessible Chromatin with high throughput Sequencing) to map the nucleosome free regions (NFRs) in the gene body of STE11 (FIG. 19G). Deletion of SET2 or a H3K36A mutation give rise to open chromatin at two NFRs, located near TATA-box like sequences (NFR1 and NFR3 in FIG. 19H). Interestingly, H3K122A gives rise to a third NFR in between the other two (NFR2, FIG. 19H), located at a B-recognition element (BRE). To expand on this finding, we examined whether set2Δ, H3K36A or H3K122A give rise to nucleosome free regions in the 11 genes (FLO8, AVO1, LCB5, SMC3, SPB4, APM2, DDC1, SYF1, OMS1, PUS4, STE11) that are evidenced to produce cryptic transcripts. Consistent with our findings at STE11, we found that all three mutants exhibit increased chromatin accessibility compared to wt (FIG. 19I).

### DISCUSSION

In summary, we show that the architectures of macromolecular assemblies can be determined using quantitative genetic interaction data collected in vivo. Remarkably, the accuracy and precision of such models are comparable to those of models based on chemical cross-linking or co-evolution analysis. A key premise of integrative modeling is that using several different types of data improves the accuracy and precision of the model. Because the pE-MAPs and CG-MAPs contain purely phenotypic measurements, collected in living cells, these datasets generate spatial restraints that are truly orthogonal to other commonly used data for integrative modeling. Because this data reflects in vivo structures, and is thus unlikely to share artifacts of biophysical methods, it could be of particularly high value in the integrative modeling process. The genetic interaction data may also allow for the characterization of assemblies that are difficult to isolate and purify or those that are only transiently stable. Importantly, the equipment required for generating these data is basic and in particular the CG-MAPs can be generated efficiently. Recent developments in CRISPR/Cas9 based approaches have paved the way for multiplexed precision genome editing in yeast (51), allowing for rapid generation of CG-MAPs. Together, these methods make feasible the proteome-wide modeling of protein complex structures, guided by global protein-protein interaction maps (52). In addition to proteins, the approach is also applicable to assemblies containing nucleic acids, thus further expanding the scope of integrative structural biology.

The relationship between phenotypic pE-MAP measurements and structure can be uncertain. The reasons for this include mutations in distant positions that are part of an allosteric network and could give rise to similar profiles, mutations that are functionally irrelevant, and mutations that perturb gene expression, mRNA stability or translation. Additionally, the approach relies on the introduction of point mutations into the proteins of interest, which may result in structural changes. However, proteins often adapt to mutations by small local changes in their structure, maintaining their overall fold and function (53). Mutations that cause major misfolding of essential proteins and/or assemblies are uncommon in pE-MAPs since the resulting fitness defects typically prevent successful screening. Thus, the method could be improved by specifically designing point mutants that do not alter the structure and/or lead to aggregation, by selecting commonly allowed mutations as determined by divergent protein sequence alignments (54).

The aim of integrative structure determination is to model the structures of macromolecular assemblies. This often requires the structures of the individual components (from X-ray crystallography, NMR, cryo-EM, comparative modeling, or increasingly, ab initio structure prediction (50, 55-57)). The quality of the structures of the individual components and input data are crucial for integrative (or indeed any other) structural approaches, and one cannot materialize a precise structure from low-quality starting structures or data. Even so, there are numerous examples of utility of structural models at lower resolution (3). For example, these models can be used to explain the architectural principles of large assemblies (58-61), describe the structural dynamics of protein complexes (62, 63), or rationalize the impact of many mutations (58). A lower resolution structure is also often a useful starting point for higher resolution structure characterization.

CRISPR/Cas9 genome editing (64) has proven highly effective for high-throughput genetic interaction mapping in mammalian cells (65, 66). To date, these efforts have relied on whole-gene perturbations, but methods for systematic generation of point mutants using CRISPR/Cas9 have recently been developed (67, 68), paving the way for mammalian pE-MAP screening. This advance provides a means for integrative structure determination of assemblies in human cells, and also allow for identification and characterization of functionally relevant structural changes that take place in disease alleles. Furthermore, several efforts are underway to generate multiscale models of entire cells (69-73). In such instances, high-throughput genetic interaction mapping could provide global insights into cellular organization and dynamics of different components, while also informing on the structures of individual assemblies.

### REFERENCES

1. F. Alber, F. Forster, D. Korkin, M. Topf, A. Sali, Integrating diverse data for structure determination of macromolecular assemblies. Annu. Rev. Biochem. 77, 443-477 (2008).
2. F. Herzog et al., Structural probing of a protein phosphatase 2A network by chemical cross-linking and mass spectrometry. Science 337, 1348-1352 (2012).
3. M. P. Rout, A. Sali, Principles for Integrative Structural Biology Studies. Cell 177, 1384-1403 (2019).
4. F. Alber et al., Determining the architectures of macromolecular assemblies. Nature 450, 683-694 (2007).
5. D. Russel et al., Putting the pieces together: integrative modeling platform software for structure determination of macromolecular assemblies. PLoSBiol. 10, e1001244 (2012).
6. A. B. Ward, A. Sali, I. A. Wilson, Biochemistry. Integrative structural biology. Science 339, 913-915 (2013).
7. F. Alber et al., The molecular architecture of the nuclear pore complex. Nature 450, 695-701 (2007).
8. K. Lasker et al., Molecular architecture of the 26S proteasome holocomplex determined by an integrative approach. Proc. Natl. Acad. Sci. U.S.A 109, 1380-1387 (2012).
9. A. Loquet et al., Atomic model of the type III secretion system needle. Nature 486, 276-279 (2012).
10. Z. Duan et al., A three-dimensional model of the yeast genome. Nature 465, 363-367 (2010).
11. J. M. Plitzko, B. Schuler, P. Selenko, Structural Biology outside the box-inside the cell. Curr. Opin. Struct. Biol. 46, 110-121 (2017).
12. M. Dimura et al., Quantitative FRET studies and integrative modeling unravel the structure and dynamics of biomolecular systems. Curr. Opin. Struct. Biol. 40, 163-185 (2016).
13. S. R. Collins, A. Roguev, N. J. Krogan, Quantitative genetic interaction mapping using the E-MAP approach. Methods Enzymol. 470, 205-231 (2010).
14. H. Braberg et al., From structure to systems: high-resolution, quantitative genetic analysis of RNA polymerase II. Cell 154, 775-788 (2013).
15. H. Braberg, E. A. Moehle, M. Shales, C. Guthrie, N. J. Krogan, Genetic interaction analysis of point mutations enables interrogation of gene function at a residue-level resolution: exploring the applications of high-resolution genetic interaction mapping of point mutations. Bioessays 36, 706-713 (2014).
16. N. Halabi, O. Rivoire, S. Leibler, R. Ranganathan, Protein sectors: evolutionary units of three-dimensional structure. Cell 138, 774-786 (2009).
17. D. S. Marks et al., Protein 3D structure computed from evolutionary sequence variation. PLoS One 6, e28766 (2011).
18. G. Diss, B. Lehner, The genetic landscape of a physical interaction. Elife 7, (2018).
19. J. M. Schmiedel, B. Lehner, Determining protein structures using deep mutagenesis. Nat. Genet. 51, 1177-1186 (2019).
20. N. J. Rollins et al., Inferring protein 3D structure from deep mutation scans. Nat. Genet. 51, 1170-1176 (2019).
21. H. Huang, S. Lin, B. A. Garcia, Y. Zhao, Quantitative proteomic analysis of histone modifications. Chemical reviews 115, 2376-2418 (2015).
22. J. Dai et al., Probing nucleosome function: a highly versatile library of synthetic histone H3 and H4 mutants. Cell 134, 1066-1078 (2008).
23. S. Jiang et al., Construction of Comprehensive Dosage-Matching Core Histone Mutant Libraries for Saccharomyces cerevisiae. Genetics 207, 1263-1273 (2017).
24. J. E. Brownell et al., Tetrahymena histone acetyltransferase A: a homolog to yeast Gcn5p linking histone acetylation to gene activation. Cell 84, 843-851 (1996).
25. L. K. Durrin, R. K. Mann, P. S. Kayne, M. Grunstein, Yeast histone H4 N-terminal sequence is required for promoter activation in vivo. Cell 65, 1023-1031 (1991).
26. H. Braberg et al., Quantitative analysis of triple-mutant genetic interactions. Nature protocols 9, 1867-1881 (2014).
27. J. E. Haber et al., Systematic triple-mutant analysis uncovers functional connectivity between pathways involved in chromosome regulation. Cell Rep 3, 2168-2178 (2013).
28. S. R. Collins, M. Schuldiner, N. J. Krogan, J. S. Weissman, A strategy for extracting and analyzing large-scale quantitative epistatic interaction data. Genome biology 7, R63 (2006).
29. T. Miller et al., COMPASS: a complex of proteins associated with a trithorax-related SET domain protein. Proc Natl Acad Sci USA 98, 12902-12907 (2001).
30. A. Roguev et al., The Saccharomyces cerevisiae Set1 complex includes an Ash2 homologue and methylates histone 3 lysine 4. EMBO J 20, 7137-7148 (2001).
31. G. Mizuguchi et al., ATP-driven exchange of histone H2AZ variant catalyzed by SWR1 chromatin remodeling complex. Science 303, 343-348 (2004).
32. M. J. Carrozza et al., Histone H3 methylation by Set2 directs deacetylation of coding regions by Rpd3S to suppress spurious intragenic transcription. Cell 123, 581-592 (2005).
33. S. Venkatesh et al., Set2 methylation of histone H3 lysine 36 suppresses histone exchange on transcribed genes. Nature 489, 452-455 (2012).
34. D. N. Reshef et al., Detecting novel associations in large data sets. Science 334, 15181524 (2011).
35. D. Albanese et al., minerva and minepy: a C engine for the MINE suite and its R, Python and MATLAB wrappers. Bioinformatics 29, 407-408 (2013).
36. C. L. White, R. K. Suto, K. Luger, Structure of the yeast nucleosome core particle reveals fundamental changes in internucleosome interactions. EMBO J. 20, 5207-5218 (2001).
37. D. Schneidman-Duhovny, Y. Inbar, R. Nussinov, H. J. Wolfson, PatchDock and SymmDock: servers for rigid and symmetric docking. Nucleic Acids Res. 33, W363-367 (2005).
38. C. J. Ryan et al., Hierarchical modularity and the evolution of genetic interactomes across species. Mol Cell 46, 691-704 (2012).
39. J. Wysocka et al., A PHD finger of NURF couples histone H3 lysine 4 trimethylation with chromatin remodelling. Nature 442, 86-90 (2006).
40. P. Shannon et al., Cytoscape: a software environment for integrated models of biomolecular interaction networks. Genome Res 13, 2498-2504 (2003).
41. T. D. Goddard et al., UCSF ChimeraX: Meeting modern challenges in visualization and analysis. Protein Sci 27, 14-25 (2018).
42. J. Han et al., A Cul4 E3 ubiquitin ligase regulates histone hand-off during nucleosome assembly. Cell 155, 817-829 (2013).
43. T. Tsubota et al., Histone H3-K56 acetylation is catalyzed by histone chaperone-dependent complexes. Mol Cell 25, 703-712 (2007).
44. Z. A. Chen et al., Architecture of the RNA polymerase II-TFIIF complex revealed by cross-linking and mass spectrometry. EMBO J. 29, 717-726 (2010).
45. S. Ovchinnikov, H. Kamisetty, D. Baker, Robust and accurate prediction of residue-residue interactions across protein interfaces using evolutionary information. Elife 3, e02030 (2014).
46. Q. Cong, I. Anishchenko, S. Ovchinnikov, D. Baker, Protein interaction networks revealed by proteome coevolution. Science 365, 185-189 (2019).
47. T. Gueudre, C. Baldassi, M. Zamparo, M. Weigt, A. Pagnani, Simultaneous identification of specifically interacting paralogs and interprotein contacts by direct coupling analysis. Proc. Natl. Acad. Sci. U.S.A 113, 12186-12191 (2016).
48. S. Wang, S. Sun, Z. Li, R. Zhang, J. Xu, Accurate De Novo Prediction of Protein Contact Map by Ultra-Deep Learning Model. PLoS Comput. Biol. 13, e1005324 (2017).
49. H. Zeng et al., ComplexContact: a web server for inter-protein contact prediction using deep learning. Nucleic Acids Res. 46, W432-W437 (2018).
50. S. Wang, S. Sun, J. Xu, Analysis of deep learning methods for blind protein contact prediction in CASP12. Proteins 86 Suppl 1, 67-77 (2018).
51. K. R. Roy et al., Multiplexed precision genome editing with trackable genomic barcodes in yeast. Nat Biotechnol 36, 512-520 (2018).
52. S. R. Collins et al., Toward a comprehensive atlas of the physical interactome of Saccharomyces cerevisiae. Mol Cell Proteomics 6, 439-450 (2007).
53. E. Eyal, R. Najmanovich, M. Edelman, V. Sobolev, Protein side-chain rearrangement in regions of point mutations. Proteins 50, 272-282 (2003).
54. R. Sasidharan, C. Chothia, The selection of acceptable protein mutations. Proc. Natl. Acad. Sci. U.S.A 104, 10080-10085 (2007).
55. J. Schaarschmidt, B. Monastyrskyy, A. Kryshtafovych, A. Bonvin, Assessment of contact predictions in CASP12: Co-evolution and deep learning coming of age. Proteins 86 Suppl 1, 51-66 (2018).
56. S. Ovchinnikov, H. Park, D. E. Kim, F. DiMaio, D. Baker, Protein structure prediction using Rosetta in CASP12. Proteins 86 Suppl 1, 113-121 (2018).
57. S. Wang, S. Sun, Z. Li, R. Zhang, J. Xu, Accurate De Novo Prediction of Protein Contact Map by Ultra-Deep Learning Model. PLoS Comput Biol 13, e1005324 (2017).
58. S. J. Kim et al., Integrative structure and functional anatomy of a nuclear pore complex. Nature 555, 475-482 (2018).
59. K. Lasker et al., Molecular architecture of the 26S proteasome holocomplex determined by an integrative approach. Proc Natl Acad Sci USA 109, 1380-1387 (2012).
60. P. J. Robinson et al., Molecular architecture of the yeast Mediator complex. Elife 4, (2015).
61. J. Luo et al., Architecture of the Human and Yeast General Transcription and DNA Repair Factor TFIIH. Mol Cell 59, 794-806 (2015).
62. C. Gutierrez et al., Structural dynamics of the human COP9 signalosome revealed by cross-linking mass spectrometry and integrative modeling. Proc Nat Acad Sci USA 117, 4088-4098 (2020).
63. K. S. Molnar et al., Cys-scanning disulfide crosslinking and bayesian modeling probe the transmembrane signaling mechanism of the histidine kinase, PhoQ. Structure 22, 12391251 (2014).
64. M. Jinek et al., A Programmable Dual-RNA-Guided DNA Endonuclease in Adaptive Bacterial Immunity. Science 337, 816-821 (2012).
65. J. P. Shen et al., Combinatorial CRISPR-Cas9 screens for de novo mapping of genetic interactions. Nat. Methods 14, 573-576 (2017).
66. D. Du et al., Genetic interaction mapping in mammalian cells using CRISPR interference. Nat. Methods 14, 577-580 (2017).
67. L. Ma et al., CRISPR-Cas9-mediated saturated mutagenesis screen predicts clinical drug resistance with improved accuracy. Proc. Natl. Acad. Sci. U.S.A 114, 1175111756 (2017).
68. A. V. Anzalone et al., Search-and-replace genome editing without double-strand breaks or donor DNA. Nature 576, 149-157 (2019).
69. S. R. McGuffee, A. H. Elcock, Diffusion, crowding & protein stability in a dynamic molecular model of the bacterial cytoplasm. PLoS Comput. Biol. 6, e1000694 (2010).
70. S. Takamori et al., Molecular anatomy of a trafficking organelle. Cell 127, 831-846 (2006).
71. B. G. Wilhelm et al., Composition of isolated synaptic boutons reveals the amounts of vesicle trafficking proteins. Science 344, 1023-1028 (2014).
72. J. Singla et al., Opportunities and Challenges in Building a Spatiotemporal Multi-scale Model of the Human Pancreatic β Cell. Cell 173, 11-19 (2018).
73. P. J. Thul et al., A subcellular map of the human proteome. Science 356, (2017).
74. A. J. Saldanha, Java Treeview—extensible visualization of microarray data. Bioinformatics 20, 3246-3248 (2004).
75. H. Braberg et al., From structure to systems: high-resolution, quantitative genetic analysis of RNA polymerase II. Cell 154, 775-788 (2013).
76. S. R. Collins et al., Functional dissection of protein complexes involved in yeast chromosome biology using a genetic interaction map. Nature 446, 806-810 (2007).
77. M. Schuldiner, S. R. Collins, J. S. Weissman, N. J. Krogan, Quantitative genetic analysis in Saccharomyces cerevisiae using epistatic miniarray profiles (E-MAPs) and its application to chromatin functions. Methods 40, 344-352 (2006).
78. W. Rieping, M. Habeck, M. Nilges, Inferential structure determination. Science 309, 303-306 (2005).
79. W. Rieping, M. Habeck, M. Nilges, Modeling errors in NOE data with a log-normal distribution improves the quality of NMR structures. J Am. Chem. Soc. 127, 1602616027 (2005).
80. H. Jeffreys, An invariant form for the prior probability in estimation problems. Proc. R. Soc. Lond. A Math. Phys. Sci. 186, 453-461 (1946).
81. D. Schneidman-Duhovny, R. Pellarin, A. Sali, Uncertainty in integrative structural modeling. Curr. Opin. Struct. Biol. 28, 96-104 (2014).
82. A. Sali et al., Outcome of the First wwPDB Hybrid/Integrative Methods Task Force Workshop. Structure 23, 1156-1167 (2015).
83. S. K. Burley et al., PDB-Dev: a Prototype System for Depositing Integrative/Hybrid Structural Models. Structure 25, 1317-1318 (2017).
84. R. Chen, J. Mintseris, J. Janin, Z. Weng, A protein—docking benchmark. Proteins: Struct. Funct. Bioinf. 52, 88-91 (2003).
85. C. M. Wood et al., High-resolution structure of the native histone octamer. Acta Crystallogr. Sect. F Struct. Biol. Cryst. Commun. 61, 541-545 (2005).
86. A. Sali, T. L. Blundell, Comparative protein modelling by satisfaction of spatial restraints. J. Mol. Biol. 234, 779-815 (1993).
87. S. M. Vos et al., Structure of activated transcription complex Pol II-DSIF-PAF-SPT6. Nature 560, 607-612 (2018).
88. M. N. Wojtas, M. Mogni, O. Millet, S. D. Bell, N. G. A. Abrescia, Structural and functional analyses of the interaction of archaeal RNA polymerase with DNA. Nucleic Acids Res. 40, 9941-9952 (2012).
89. P. Cramer, D. A. Bushnell, R. D. Kornberg, Structural basis of transcription: RNA polymerase II at 2.8 Angstrom resolution. Science, (2001).
90. K. S. Murakami, X-ray crystal structure of Escherichia coli RNA polymerase 670 holoenzyme. J. Biol. Chem. 288, 9126-9134 (2013).
91. R. P. Joosten et al., A series of PDB related databases for everyday needs. Nucleic Acids Res. 39, D411-419 (2011).
92. W. Kabsch, C. Sander, Dictionary of protein secondary structure: pattern recognition of hydrogen-bonded and geometrical features. Biopolymers 22, 2577-2637 (1983).
93. M.-Y. Shen, A. Sali, Statistical potential for assessment and prediction of protein structures. Protein Sci. 15, 2507-2524 (2006).
94. J. P. Erzberger et al., Molecular architecture of the 40S-eIF1-eIF3 translation initiation complex. Cell 158, 1123-1135 (2014).
95. Y. Shi et al., Structural characterization by cross-linking reveals the detailed architecture of a coatomer-related heptameric module from the nuclear pore complex. Mol. Cell. Proteomics 13, 2927-2943 (2014).
96. R. H. Swendsen, J. S. Wang, Replica Monte Carlo simulation of spin glasses. Phys. Rev. Lett. 57, 2607-2609 (1986).
97. S. Viswanath, I. E. Chemmama, P. Cimermancic, A. Sali, Assessing Exhaustiveness of Stochastic Sampling for Integrative Modeling of Macromolecular Structures. Biophys. J. 113, 2344-2353 (2017).
98. B. Vallat, B. Webb, J. D. Westbrook, A. Sali, H. M. Berman, Development of a Prototype System for Archiving Integrative/Hybrid Structure Models of Biological Macromolecules. Structure 26, 894-904.e892 (2018).
99. J. D. Chodera, A Simple Method for Automated Equilibration Detection in Molecular Simulations. J. Chem. Theory Comput. 12, 1799-1805 (2016).
100. L. McInnes, J. Healy, S. Astels, hdbscan: Hierarchical density based clustering. The Journal of Open Source Software 2, 205 (2017).
101. E. D. Merkley et al., Distance restraints from crosslinking mass spectrometry: mining a molecular dynamics simulation database to evaluate lysine-lysine distances. Protein Sci. 23, 747-759 (2014).
102. D. Schneidman-Duhovny et al., A method for integrative structure determination of protein-protein complexes. Bioinformatics 28, 3282-3289 (2012).
103. M. J. de Hoon, S. Imoto, J. Nolan, S. Miyano, Open source clustering software. Bioinformatics 20, 1453-1454 (2004).
104. J. T. Robinson et al., Integrative genomics viewer. Nat Biotechnol 29, 24-26 (2011).
105. J. H. Morris, C. C. Huang, P. C. Babbitt, T. E. Ferrin, structureViz: linking Cytoscape and UCSF Chimera. Bioinformatics 23, 2345-2347 (2007).
106. N. T. Doncheva, K. Klein, F. S. Domingues, M. Albrecht, Analyzing and visualizing residue networks of protein structures. Trends Biochem Sci 36, 179-182 (2011).
107. J. H. Morris et al., setsApp for Cytoscape: Set operations for Cytoscape Nodes and Edges. F1000Res 3, 149 (2014).
108. M. A. Collart, S. Oliviero, Preparation of yeast RNA. Curr Protoc Mol Biol Chapter 13, Unit13 12 (2001).
109. D. Kim et al., TopHat2: accurate alignment of transcriptomes in the presence of insertions, deletions and gene fusions. Genome biology 14, R36 (2013).
110. S. Anders, P. T. Pyl, W. Huber, HTSeq—a Python framework to work with high-throughput sequencing data. Bioinformatics 31, 166-169 (2015).
111. M. I. Love, W. Huber, S. Anders, Moderated estimation of fold change and dispersion for RNA-seq data with DESeq2. Genome biology 15, 550 (2014).
112. M. Costanzo et al., The genetic landscape of a cell. Science 327, 425-431 (2010).
113. G. M. Wilmes et al., A genetic interaction map of RNA-processing factors reveals links between Sem1/Dss1-containing complexes and mRNA export and splicing. Mol Cell 32, 735-746 (2008).
114. Y. H. Benjamini, Y, Controlling the False Discovery Rate: A Practical and Powerful Approach to Multiple Testing. Journal of the Royal Statistical Society. Series B 57, 289300 (1995).
115. J. Cox, M. Mann, MaxQuant enables high peptide identification rates, individualized p.p.b.-range mass accuracies and proteome-wide protein quantification. Nat Biotechnol 26, 1367-1372 (2008).
116. B. MacLean et al., Skyline: an open source document editor for creating and analyzing targeted proteomics experiments. Bioinformatics 26, 966-968 (2010).
117. D. Wessel, U. I. Flugge, A method for the quantitative recovery of protein in dilute solution in the presence of detergents and lipids. Anal Biochem 138, 141-143 (1984).
118. M. Downey et al., Acetylome profiling reveals overlap in the regulation of diverse processes by sirtuins, gcn5, and esa1. Mol Cell Proteomics 14, 162-176 (2015).
119. M. Choi et al., MSstats: an R package for statistical analysis of quantitative mass spectrometry-based proteomic experiments. Bioinformatics 30, 2524-2526 (2014).
120. K. J. Livak, T. D. Schmittgen, Analysis of relative gene expression data using real-time quantitative PCR and the 2(-Delta C(T)) Method. Methods 25, 402-408 (2001).
121. R. Dronamraju, B. D. Strahl, A feed forward circuit comprising Spt6, Ctk1 and PAF regulates Pol II CTD phosphorylation and transcription elongation. Nucleic Acids Res 42, 870-881 (2014).
122. Y. Liao, G. K. Smyth, W. Shi, featureCounts: an efficient general purpose program for assigning sequence reads to genomic features. Bioinformatics 30, 923-930 (2014).
123. E. M. Hyland et al., Insights into the role of histone H3 and histone H4 core modifiable residues in Saccharomyces cerevisiae. Mol Cell Biol 25, 10060-10070 (2005).
124. H. Masumoto, D. Hawke, R. Kobayashi, A. Verreault, A role for cell-cycle-regulated histone H3 lysine 56 acetylation in the DNA damage response. Nature 436, 294-298 (2005).
125. M. Giannattasio, F. Lazzaro, P. Plevani, M. Muzi-Falconi, The DNA damage checkpoint response requires histone H2B ubiquitination by Rad6-Bre1 and H3 methylation by Dot1. J Biol Chem 280, 9879-9886 (2005).
126. I. Celic et al., The sirtuins hst3 and Hst4p preserve genome integrity by controlling histone h3 lysine 56 deacetylation. Curr Biol 16, 1280-1289 (2006).
127. I. Celic, A. Verreault, J. D. Boeke, Histone H3 K56 hyperacetylation perturbs replisomes and causes DNA damage. Genetics 179, 1769-1784 (2008).
128. C. D. Kaplan, L. Laprade, F. Winston, Transcription elongation factors repress transcription initiation from cryptic sites. Science 301, 1096-1099 (2003).
129. H. N. Du, S. D. Briggs, A nucleosome surface formed by histone H4, H2A, and H3 residues is needed for proper histone H3 Lys36 methylation, histone acetylation, and repression of cryptic transcription. J Biol Chem 285, 11704-11713 (2010).
130. M. Simon et al., Histone fold modifications control nucleosome unwrapping and disassembly. Proc Natl Acad Sci USA 108, 12711-12716 (2011).
131. S. Chen et al., Structure-function studies of histone H3/H4 tetramer maintenance during transcription by chaperone Spt2. Genes Dev 29, 1326-1340 (2015).
132. H. G. Tran, D. J. Steger, V. R. Iyer, A. D. Johnson, The chromo domain protein chd1p from budding yeast is an ATP-dependent chromatin-modifying factor. EMBO J 19, 23232331 (2000).
133. B. N. Devaiah et al., BRD4 is a histone acetyltransferase that evicts nucleosomes from chromatin. Nat Struct Mol Biol 23, 540-548 (2016).
134. M. S. Cosgrove, J. D. Boeke, C. Wolberger, Regulated nucleosome mobility and the histone code. Nat Struct Mol Biol 11, 1037-1043 (2004).
135. J. Schneider, P. Bajwa, F. C. Johnson, S. R. Bhaumik, A. Shilatifard, Rtt109 is required for proper H3K56 acetylation: a chromatin mark associated with the elongating RNA polymerase II. J Biol Chem 281, 37270-37274 (2006).

1. A method of identifying a protein-protein interaction associated with a disorder, said method comprising:
a) selecting a first nucleic acid sequence associated with the disorder and a second nucleic acid sequence, wherein the second nucleic acid sequence is a wild-type sequence relative to the first nucleic acid sequence and associated with a non-disordered phenotype, and wherein at least the first nucleic acid sequence comprises a point mutation relative to the second nucleic acid sequence;
b) creating a point-mutant epistatic miniarray profile (pE-MAP) or a chemical genetics miniarray profile (CG-MAP) associated with the first nucleic acid sequence;
c) calculating an S-score associated with the first nucleic acid sequence and a third nucleic acid sequence;
d) calculating a maximal information coefficient (MIC) associated with the first nucleic acid sequence relative to the distance in nucleic acid number between the first nucleic acid sequence and the third nucleic acid sequence relative to the position of the third nucleic acid sequence position on the genome of a subject;
e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and
f) mapping a protein-protein interaction as between the amino acid sequence encoded by the first nucleic acid sequence and the amino acid sequence encoded by the third nucleic acid sequence.

2-5. (canceled)

6. The method of claim 1, wherein the correlating of step (e) comprises calculating a Pearson correlation.

7. The method of claim 1, wherein the correlating of step (e) comprises:
i) calculating a plurality of MICs;
ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence;
iii) performing a noise model for calculation of a standard deviation; and
iv) formulating spatial restraints as Bayesian data likelihoods.

8. The method of claim 7, wherein the upper distance bound is calculated by:
a) binning the plurality of MICs into 20 intervals;
b) selecting a maximum distance spanned by any pair of amino acid residues in each bin; and
c) fitting a logarithmic decay function (dᵤ) to the upper distance bound:
$${d}_{U} ( MIC ) = \{ \begin{matrix}\frac{log ( MIC ) - n}{k} & if MIC \leq 0.6 \\ 20 & if MIC > 0.6\end{matrix} \qquad ( 1 )$$
wherein n is −0.0147 and k is −0.41.

9. The method of claim 7, wherein the spatial restraints are formulated as Bayesian data likelihoods by calculating a posterior probability of model M given data D and prior information I by:
p(M|D,J)∝p(D|M,J)·p(M|I)
wherein the model, M, consists of a structure X and unknown parameters Y,
prior p(M|I) is the probability density of model M given I, and
likelihood function p(D|M, I) is the probability density of observing data D given M and I.

10. The method of claim 1, further comprising calculating the likelihood of the entire pE-MAP or CG-MAP dataset, a product over the individual observations between residue pairs i, j:
p(D|M,I)=Π_{i,j}N(d_{i,j}|f_{i,j}(X),σ_{i,j})
wherein f_{i,j}(X) is a forward model that predicts the data point d_{i,j} in D that would have been observed for structure X in an experiment without noise, and is defined as:
$${f}_{i , j} ( X ) = MIC ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$
wherein d₀=dᵤ (0.6);
wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:
$$P ( {MIC}_{i , j}^{obs} \vert MICi , j , X , {σ}_{i , j} ) = \{ \begin{matrix}\frac{1}{N} & if {MIC}_{i , j}^{obs} \geq {MIC}_{i , j} \\ \frac{1}{M} \frac{1}{\sqrt{2 {πσ}_{i , j}^{2}} {MIC}_{i , j}^{obs}} exp ( - \frac{1}{2 {σ}_{i , j}^{2}} {log}^{2} ( \frac{{MIC}_{i , j}^{obs}}{{MIC}_{i , j}} ) ) & if {MIC}_{i , j}^{obs} < {MIC}_{i , j}\end{matrix} \qquad ( 3 )$$
wherein σ_{i,j} are the noise parameters that can optionally be determined as part of the model, and N and M are normalization factors necessary to make the likelihood continuous.

11-12. (canceled)

13. The method of claim 1, wherein the mapping of the protein-protein interaction has a resolution from about 1 to about 10 angstroms.

14. A method of determining structure of a protein-protein interaction, said method comprising:
a) selecting a first nucleic acid sequence and a second nucleic acid sequence, wherein the second nucleic acid sequence is a wild-type sequence relative to the first nucleic acid sequence, and wherein at least the first nucleic acid sequence comprises a point mutation relative to the second nucleic acid sequence;
b) creating a point-mutant epistatic miniarray profile (pE-MAP) or a chemical genetics miniarray profile (CG-MAP) associated with the first nucleic acid sequence;
c) calculating an S-score associated with the first nucleic acid sequence and the third nucleic acid sequence;
d) calculating a maximal information coefficient (MIC) associated with the first nucleic acid sequence relative to the distance in nucleic acid number between the first nucleic acid sequence and a third nucleic acid sequence relative to the position of the third nucleic acid sequence position on the genome of a subject;
e) correlating the MIC and the pE-MAP, or the MIC and the CG-MAP, with: (i) spatial positions of amino acid residues within an amino acid sequence encoded by the first nucleic acid sequence; and (ii) spatial positions of amino acid residues within an amino acid sequence encoded by the third nucleic acid sequence; and
f) mapping a protein-protein interaction as between the amino acid sequence encoded by the first nucleic acid sequence and the amino acid sequence encoded by the third nucleic acid sequence.

15. (canceled)

16. The method of claim 14, wherein the correlating of step (e) comprises:
i) calculating a plurality of MICs;
ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence;
iii) performing a noise model for calculation of a standard deviation; and
iv) formulating spatial restraints as Bayesian data likelihoods.

17. The method of claim 16, wherein the upper distance bound is calculated by:
a) binning the plurality of MICs into 20 intervals;
b) selecting a maximum distance spanned by any pair of amino acid residues in each bin; and
c) fitting a logarithmic decay function (dᵤ) to the upper distance bound:
$${d}_{U} ( MIC ) = \{ \begin{matrix}\frac{log ( MIC ) - n}{k} & if MIC \leq 0.6 \\ 20 & if MIC > 0.6\end{matrix} \qquad ( 1 )$$
wherein n is −0.0147 and k is −0.41.

18. (canceled)

19. The method of claim 14, further comprising calculating the likelihood of the entire pE-MAP or CG-MAP dataset, a product over the individual observations between residue pairs i, j:
p(D|M,I)=Π_{i,j}N(d_{i,j}|f_{i,j}(X),σ_{i,j})
wherein f_{i,j}(X) is a forward model that predicts the data point d_{i,j} in D that would have been observed for structure X in an experiment without noise, and is defined as:
$${f}_{i , j} ( X ) = MIC ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$
wherein d₀=dᵤ (0.6);
wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:
$$P ( {MIC}_{i , j}^{obs} \vert MICi , j , X , {σ}_{i , j} ) = \{ \begin{matrix}\frac{1}{N} & if {MIC}_{i , j}^{obs} \geq {MIC}_{i , j} \\ \frac{1}{M} \frac{1}{\sqrt{2 {πσ}_{i , j}^{2}} {MIC}_{i , j}^{obs}} exp ( - \frac{1}{2 {σ}_{i , j}^{2}} {log}^{2} ( \frac{{MIC}_{i , j}^{obs}}{{MIC}_{i , j}} ) ) & if {MIC}_{i , j}^{obs} < {MIC}_{i , j}\end{matrix} \qquad ( 3 )$$
wherein σ_{i,j} are the noise parameters that can optionally be determined as part of the model, and N and M are normalization factors necessary to make the likelihood continuous.

20-21. (canceled)

22. The method of claim 14, wherein the mapping of the protein-protein interaction has a resolution from about 1 to about 10 angstroms.

23-31. (canceled)

32. A computer program product encoded on a computer-readable storage medium, wherein the computer program product comprises instructions for:
a) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence;
b) calculating an S-score associated with the first nucleic acid sequence and the third nucleic acid sequence;
c) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and
d) mapping a protein-protein interaction as between the amino acid sequence encoded by the first nucleic acid sequence and the amino acid sequence encoded by the third nucleic acid sequence.

33. The computer program product of claim 32, wherein the correlating of step (c) comprises:
i) calculating a plurality of MICs;
ii) calculating an upper distance bound between the spatial positions of the amino acid residues within the amino acid sequence encoded by the first nucleic acid sequence and the spatial positions of the amino acid residues within the amino acid sequence encoded by the third nucleic acid sequence;
iii) performing a noise model for calculation of a standard deviation; and
iv) formulating spatial restraints as Bayesian data likelihoods.

34. The computer program product of claim 33, wherein the upper distance bound is calculated by:
a) binning the plurality of MICs into 20 intervals;
b) selecting a maximum distance spanned by any pair of amino acid residues in each bin; and
c) fitting a logarithmic decay function (dᵤ) to the upper distance bound:
$${d}_{U} ( MIC ) = \{ \begin{matrix}\frac{log ( MIC ) - n}{k} & if MIC \leq 0.6 \\ 20 & if MIC > 0.6\end{matrix} \qquad ( 1 )$$
wherein n is −0.0147 and k is −0.41.

35. The computer program product of claim 33, wherein the spatial restraints are formulated as Bayesian data likelihoods by calculating a posterior probability of model M given data D and prior information I by:
wherein the model, M, consists of a structure X and unknown parameters Y,
prior p(M|I) is the probability density of model M given I, and
likelihood function p(D|M, I) is the probability density of observing data D given M and I.

36. The computer program product of claim 32, further comprising instructions for calculating the likelihood of the entire pE-MAP or CG-MAP dataset, a product over the individual observations between residue pairs i, j:
p(D|M,I)=Π_{i,j}N(d_{i,j}|f_{i,j}(X),σ_{i,j})
wherein f_{i,j}(X) is a forward model that predicts the data point d_{i,j} in D that would have been observed for structure X in an experiment without noise, and is defined as:
$${f}_{i , j} ( X ) = MIC ( {d}_{i , j} ) = \{ \begin{matrix}exp ( k \cdot {d}_{i , j} + n ) & if {d}_{i , j} \leq {d}_{0} \\ 0.6 & if {d}_{i , j} > {d}_{0}\end{matrix} \qquad ( 2 )$$
wherein d₀=dᵤ (0.6);
wherein N(d_{i,j}|f_{i,j}(X),σ_{i,j}) is a noise model that quantifies the deviation between the predicted and observed data points and is a lognormal distribution with a flat plateau for MIC values below the upper bound on the observed MIC values:
$$P ( {MIC}_{i , j}^{obs} \vert MICi , j , X , {σ}_{i , j} ) = \{ \begin{matrix}\frac{1}{N} & if {MIC}_{i , j}^{obs} \geq {MIC}_{i , j} \\ \frac{1}{M} \frac{1}{\sqrt{2 {πσ}_{i , j}^{2}} {MIC}_{i , j}^{obs}} exp ( - \frac{1}{2 {σ}_{i , j}^{2}} {log}^{2} ( \frac{{MIC}_{i , j}^{obs}}{{MIC}_{i , j}} ) ) & if {MIC}_{i , j}^{obs} < {MIC}_{i , j}\end{matrix} \qquad ( 3 )$$
wherein σ_{i,j} are the noise parameters that can optionally be determined as part of the model, and N and M are normalization factors necessary to make the likelihood continuous.

37-40. (canceled)

41. A system comprising the computer program product of claim 32, and one or more of:
a) a processor operable to execute programs; and
b) a memory associated with the processor.

42. A system for identifying a protein interaction network in a subject, the system comprising:
a) a processor operable to execute programs;
b) a memory associated with the processor;
c) a database associated with said processor and said memory; and
d) a program stored in the memory and executable by the processor, the program being operably for:
i) calculating a maximal information coefficient (MIC) associated with a first nucleic acid sequence relative to the distance in nucleic acid number between the first nucleic acid sequence and a third nucleic acid sequence relative to the position of the third nucleic acid sequence position on the genome of a subject, wherein the first nucleic acid sequence comprises a point mutation relative to a second sequence which is a wild-type sequence relative to the first nucleic acid sequence;
ii) calculating an S-score associated with the first nucleic acid sequence and the third nucleic acid sequence;
iii) correlating the MIC and a point-mutant epistatic miniarray profile (pE-MAP) or a chemical genetics miniarray profile (CG-MAP) associated with the first nucleic acid sequence with: (i) spatial positions of amino acid residues within an amino acid sequence encoded by the first nucleic acid sequence; and (ii) spatial positions of amino acid residues within an amino acid sequence encoded by the third nucleic acid sequence; and
iv) mapping a protein-protein interaction as between the amino acid sequence encoded by the first nucleic acid sequence and the amino acid sequence encoded by the third nucleic acid sequence.

43-46. (canceled)

47. A method of creating a genetic interaction profile, said method comprising:
a) creating a point-mutant epistatic miniarray profile (pE-MAP) or a chemical genetics miniarray profile (CG-MAP) associated with a first nucleic acid sequence and a second nucleic acid sequence;
b) calculating an S-score associated with the first nucleic acid sequence and the second nucleic acid sequence; and
c) correlating the pE-MAP and the S-score, or the CG-MAP and the S-score, to create the genetic interaction profile between the first nucleic acid sequence and the second nucleic acid sequence.

48. (canceled)
